(19) United States
(12) Patent Application Publication
(10) Pub. No.: US 2024/0342301 A1
(43) Pub. Date: Oct. 17, 2024

(54) ANTIBODY-DRUG CONJUGATES

(71) Applicants: MC SAF, Tours (FR); UNIVERSITE DE TOURS, Tours Cedex 1 (FR); CENTRE HOSPITALIER REGIONAL UNIVERSITAIRE DE TOURS, Tours Cedex 9 (FR)

(72) Inventors: Camille MARTIN, Tours (FR); Ofelia FEUILLÂTRE, Tours (FR); Yves GRUEL, Tours (FR); Jérôme ROLLIN, ROCHECORBON (FR)

(21) Appl. No.: 18/294,636

(22) PCT Filed: Aug. 4, 2022
(86) PCT No.: PCT/FR2022/051554

§ 371 Date: Feb. 2, 2024

(30) Foreign Application Priority Data
Aug. 5, 2021 (FR) FR2108537

(51) Int. Cl. A61K 47/68; C07K 16/32; C12N 9/52

(52) U.S. Cl. CPC A61K 47/6815; A61K 47/6855; A61K 47/6889; C07K 16/32; C12N 9/52; C07K 2317/55

(57) ABSTRACT

The invention relates to a conjugate containing a) an antibody or fragment of an antibody, and b) a specific protease for the immunoglobulin G (IgG) hinge region. The Invention also relates to a composition comprising at least one such conjugate, as well as to the use of this conjugate as a drug, in particular for the treatment of a disease caused by autoantibodies.

### TECHNICAL FIELD

The present invention relates to conjugates comprising an antibody or an antibody fragment and a specific protease of the hinge region of immunoglobulins G (IgG), said conjugates being able to be used as drugs, particular in the treatment of autoimmune diseases induced by immunoglobulins G (IgG).

### PRIOR ART

Autoimmune diseases are characterised by an abnormal reaction of the immune system which then attacks the components of the “self”. The immune system of the patient then generates pathogenic autoantibodies (immunoglobulins G) which will, by recruitment and/or activation of various immune compartments, lead to the destruction of healthy cells essential for proper physiological functioning. This disruption mostly takes place via the intermediary of the effector functions of the antibodies mediated by interactions between the Fc portion of these antibodies and Fc receptors (FcR) present on various cells of the immune system.

The origin of autoimmune diseases remains poorly understood. They are due to a combination of several factors such as a genetic predisposition, taking of certain drugs, exposure to infectious agents or even environmental factors.

The therapeutic options currently available are:
intravenous injection of polyvalent immunoglobulins (IVIg) in order to saturate the FcR receptors and thus prevent the autoantibodies from using their effector functions;
using anti-CD20 antibodies, such as rituximab monoclonal antibody, which will deplete the organism of lymphocyte B, which are the cells producing antibodies;
splenectomy, which consists of ablation of the liver, an organ involved in the maturation and storage of lymphocytes.

The use of anti-CD20 antibody and splenectomy are more suitable for chronic cases. Although effective, these strategies have disadvantages. More specifically, the use of anti-CD20 antibody and the splenectomy lead to a massive depletion in androgenic antibodies, which can make the patient susceptible to infections. With regard to the use of IVIg, this remains limited since the supply is dependent on blood plasma donations.

Experimental treatments have also been tested for treating pathologies linked to autoantibodies. Thus, the ability of the bacterial protease IdeS, a protease capable of cutting immunoglobulins G in their hinge region, was tested with success in the context of a renal graft with non-compatible donors. The injection of IdeS led to a drastic reduction in circulating immunoglobulins and to the temporary maintenance of the graft. However, new injections of IdeS, necessary for the survival of the graft, induce an immune response against protease IdeS, annulling its effect. Moreover, since the half-life of this protease in humans is several hours, its action remains temporary. This same strategy has also been applied to a particular autoimmune disease, heparin-induced thrombocytopenia, and has demonstrated, in an animal model, a complete proteolysis of the circulating immunoglobulins, thus annulling the pathogenic action of the autoantibodies. However, the complete disappearance of endogenous immunoglobulins generated an immune deficiency.

There is therefore a need to develop novel targeted therapies in order to be able to effectively treat autoimmune diseases induced by pathogenic immunoglobulins G, by overcoming the inherent constraints of the known treatments.

It is in this context that the inventors have developed a novel drug which is in the form of a conjugate comprising an antibody or an antibody fragment and a specific protease of the hinge region of immunoglobulins G (IgG). This conjugate can target the protease at the site of action of the pathogenic autoantibodies, which makes it possible to specifically destroy them. The inventors have developed, in particular, a chemistry particularly suitable for the preparation of conjugates comprising an antibody or an antibody fragment and a specific protease of the hinge region of immunoglobulins G (IgG).

### SUMMARY OF THE INVENTION

The present invention relates to a conjugate comprising:
a) an antibody or an antibody fragment, and
b) a specific protease of the hinge region of immunoglobulins G (IgG).

The present invention also relates to a conjugate according to the invention, for use as a drug, in particular in the treatment of a disease induced by autoantibodies.

### DESCRIPTION OF THE FIGURES

FIG. 1 shows the kinetics of recognition of the antigen HER2 by TTZ Fab and compound 24.

### DETAILED DESCRIPTION

### Definitions

In the context of the present invention, the terms “antibody” and “immunoglobulin” designate a heterotetramer consisting of two heavy chains of approximately 50-70 kDa each (referred to as H chains, for Heavy) and two light chains of approximately 25 kDa each (referred to as L chains for Light), linked together by intrachain and interchain disulfide bridges. Each chain consists of, in the N-terminal position, a region or variable domain, called VL for the light chain, VH for the heavy chain and, in the C-terminal position, a constant region consisting of a single domain, called CL for the light chain and three or four domains named CH1, CH2, CH3, CH4, for the heavy chain.

Within the meaning of the invention, the term “antibody fragment” shall mean any portion of an antibody, capable of bonding to an antigen, obtained by enzymatic digestion or obtained by bioproduction, and comprising at least one disulfide bridge. It may be, for example, a fragment chosen from Fab, Fab′, F (ab′₂ and scFv-Fc.

The enzymatic digestion of immunoglobulins by papain generates two identical fragments which are called the Fab fragments (antigen binding fragment), and an Fc fragment (crystallisable fragment). The enzymatic digestion of immunoglobulins by pepsin generates an F (ab′₂ fragment and an Fc fragment split into several peptides. F (ab′₂ is formed of two Fab′ fragments linked by interchain disulfide bridges. The Fab portions consist of variable regions and CH1 and CL domains. The Fab′ fragment consists of the Fab region and a hinge region. The scFv (single-chain variable fragment) is a fragment coming from the engineering of proteins which consists uniquely of variable VH and VL domains. The structure is stabilised by a short flexible peptide arm, called a linker, which is placed between the two domains. Two scFv fragments can be linked to an Fc fragment in order to give an scFv-Fc.

The terms “purified” and “isolated” shall mean, when referring to a conjugate according to the invention, that the conjugate is present in the substantial absence of other biological macromolecules of the same type. The term “purified” as used here, preferably signifies at least 75% by mass, more preferably at least 85% by mass, still more preferably at least 95% by mass, and most preferably at least 98% by mass of conjugate, relative to all of the macromolecules present.

The term “pharmaceutically acceptable” means approved by a federal or state regulatory agency, or listed in the American or European pharmacopoeia, or in another generally recognised pharmacopoeia, and applies to a product intended to be used in animals and/or in humans. A “pharmaceutical composition” designates a composition comprising a pharmaceutically acceptable carrier. For example, a pharmaceutically acceptable carrier can be a diluent, an adjuvant, an excipient or a carrier with which the therapeutic agent is administered. These carriers can be sterile liquids, such as water and oils, including those of petroleum/animal, plant or synthetic origin, such as peanut oil, soybean oil, mineral oil, sesame oil, etc. Water is a preferred carrier when the pharmaceutical composition is administered intravenously. Saline solutions and aqueous solutions of dextrose and glycerol can also be used as liquid carriers, in particular for injectable solutions. Pharmaceutically acceptable excipients include starch, glucose, lactose, sucrose, sodium stearate, glycerol monostearate, talc, sodium chloride, powdered skimmed milk, glycerol, propylene glycol, water, ethanol and the like. When the pharmaceutical composition is suitable for oral administration, the tablets or soft capsules can be prepared by conventional means with pharmaceutically acceptable excipients such as binders (for example pregelatinised corn starch, polyvinylpyrrolidone or hydroxypropyl methylcellulose); fillers (for example lactose, microcrystalline cellulose or calcium hydrogen phosphate); lubricants (for example, magnesium stearate, talc or silica); disintegrating agents (for example potato starch or sodium starch glycolate); or wetting agents (for example, sodium laurylsulfate). The tablets can be coated by processes that are well known in the state of the art. The liquid preparations for oral administration can take the form, for example, of solutions, syrups or suspensions, or can be presented in the form of dry product to be reconstituted with water or another appropriate carrier, before use. Such liquid preparations can be prepared by conventional means using pharmaceutically acceptable carriers such as suspension agents (for example a syrup of sorbitol, cellulose derivatives or hydrogenated edible fats); emulsifiers (for example, lecithin or acacia); non-aqueous carriers (for example almond oil, oil esters, ethyl alcohol or fractionated vegetable oils); and preservatives (for example methyl, propyl or sorbic acid p-hydroxybenzoates). The pharmaceutical compositions can also contain buffer salts, flavours, colourings and sweeteners, as required. The composition according to the invention is preferably a pharmaceutical composition.

The term “treat” or “treatment” encompasses any beneficial or desirable effect on a pathology or a pathological state, and can also include a minimum reduction of one or more measurable markers of the pathology or pathological state. The treatment can involve, for example, either the reduction of or improvement in symptoms of the pathology or pathological state, or slowing of the progress of the disease or pathological state. The term “treatment” does not necessarily signify the complete eradication or cure of the pathology, nor the associated symptoms.

The term “native mass spectrometry” shall mean a mass spectrometry analysis carried out under conditions that do not denature the proteins, thus enabling the non-covalent bonds to be preserved. Native mass spectrometry methods are widely described in the literature, for example in reference [1].

The term “protease” (or peptidase or proteolytic enzymes) shall mean enzymes which cut the peptide bonds of proteins. This is then referred to as proteolytic cutting or proteolysis. This process involves the use of a water molecule which means that it is classified as a hydrolase. Thus, a “specific protease of the immunoglobulin G (IgG) hinge region” is a protease which cuts the hinge region of IgG. Several proteases specific to the immunoglobulin G (IgG) hinge region are described in the literature and can be used in the context of the present invention.

The term “aryl” shall mean a phenyl or naphtyl group.

The term “saturated, unsaturated or partially unsaturated heterocycle, having 5 to 15 members, and comprising 1 to 4 heteroatoms chosen from nitrogen, oxygen and sulfur” shall mean a monocyclic, bicyclic or tricyclic, optionally fused, saturated, unsaturated or partially unsaturated group, comprising 1 to 4 heteroatoms, preferably 1 to 3 heteroatoms, and more preferably 1 or 2 heteroatoms, chosen from nitrogen, oxygen and sulfur.

Examples of unsaturated monocycles include pyrrolyl, pyrazolyl, imidazolyl, oxazolyl, isoxazolyl, triazolyl, oxadiazolyl, furanyl, thienyl, thiazolyl, isothiazolyl, thiadiazolyl, pyridyl, pyridazinyl, pyrimidinyl, pyrazinyl, triazinyl, azepinyl, oxepinyl or thiepinyl groups.

Examples of saturated monocycles include pyrrolidinyl, tetrahydrofuryl, tetrahydrothienyl, imidazolidinyl, thiazolidinyl, isoxazolidinyl, piperidinyl, piperazinyl, morpholinyl, thiomorpholinyl, or hexahydroazepinyl groups.

An example of a partially unsaturated monocycle is the dihydro (is) oxazol group.

Examples of unsaturated or partially unsaturated, optionally fused, bicycles or tricycles include the groups isoquinolyl, quinolyl, 1,4-dihydroquinolinyl, 2,4-dihydroquinolinyl, 1,2,3,4-tetrahydroquinolinyl, 1/pyrrolo[3,2-b]pyridinyl, benzimidazolyl, benzopyrazinyl, indolyl, 2,3-dihydroindolyl, indolynyl, benzofuranyl, 2,3-dihydrobenzofuranyl, benzothiazolyl, benzothiadiazolyl, benzisoxazolyl, 3,4-dihydro-1,4-benzoxazinyl, 2,4-dihydro-1,4-benzoxazinyl, 1,3-benzodioxolyl, 2,3-dihydrobenzodioxinyl, imidazothiazolyl, benzoxazolyl, benzoxazinyl, 4,5-dihydro-1,5-benzoxazepinyl, 2,3-dihydropyrido[4,3-b] [1,4]oxazinyl, 3,4-dihydropyrido[3,2-b][1,4]oxazinyl, spiro [benzoxazine-2,1′-cyclobutane]-yl, chromanyl, chromenyl, spiro [chromane-2,1′-cyclobutane], spiro [chromene-2,1′-cyclobutane], spiro [cyclopentane-1,3′-indoline]-yl, spiro [indoline-3,3′-tetrahydrofurane]-yl, spiro [indoline-3,3′-tetrahydropyrane]-yl, dihydrocyclopropa [b] indol-2-yl, hexahydrocarbazolyl, tetrahydrocarbazolyl, dihydrocarbazolyl or tetrahydrocyclopenta [b] indol-4-yl.

### Conjugate

A first object of the present invention relates to a conjugate comprising:
a) an antibody or an antibody fragment, and
b) a specific protease of the hinge region of immunoglobulins G (IgG).

A person skilled in the art will know that the generic term “conjugate” can be substituted by the generic expression “antibody-drug conjugate” or “ADC”.

In the context of the present invention, the antibody can be of mammal origin (e.g. human or mouse), humanised or chimeric. It is preferably a monoclonal antibody produced through recombination by genetically modified cells according to techniques that are widely described in the prior art. It may involve a generic, humanised or human, monospecific or bispecific monoclonal antibody. The antibody can be an IgG, for example an IgG1, IgG2, IgG3 or IgG4. Thus, the antibody fragment can be an IgG fragment, for example a fragment of IgG1, IgG2, IgG3 or IgG4.

When the protease is conjugated with an antibody or an antibody fragment comprising an Fc fragment (e.g. an scFv-Fc), the half-life in blood of the conjugate can be greater than the half-life of the non-conjugated protease with the antibody or an antibody fragment comprising an Fc fragment. More specifically, the Fc fragment is known to have a long half-life in blood and the conjugate according to the invention can therefore benefit from the half-life of the Fc fragment.

In the context of the present invention, the antibody fragment can be chosen from Fab, Fab′ and F (ab′₂, preferably chosen from Fab and F (ab′₂. An antibody fragment chosen from Fab, Fab′ and F (ab′₂, preferably chosen from Fab and F (ab′₂, is particularly advantageous in the context of the present invention.

In an embodiment, the conjugate according to the invention does not cleave the hinge region of another conjugate according to the invention. For example, the amino acid sequence of the antibody is mutated, for example at the hinge region, so that the conjugate according to the invention does not cleave the hinge region of another conjugate according to the invention. Said amino acid sequence may comprise, for example, at least 1 mutation such as a substitution, a deletion or an addition. Such mutations are widely described in the literature [3].

In the context of the present invention, the protease can be chosen from IdeS, IdeZ, IdeE, IgdE, SeMac, preferably IdeS.

In an embodiment, the protease is bonded at a disulfide bridge of the antibody or of the antibody fragment.

The conjugate of the invention is capable of bonding the antigen recognised by the antibody or the antibody fragment and has a protease activity of the hinge region of the IgG. Thus, the conjugate according to the invention can specifically target the protease at the point where the antigen is located. The antibody or the antibody fragment will then be chosen according to the desired targeting of the protease in the organism. For example, the antigen recognised by the conjugate of the invention can be chosen from:
platelet factor 4 (PF4), for example in the treatment of heparin-induced thrombocytopenias (HIT) and the vaccine-induced thrombotic thrombocytopenia (VITT);
glycoprotein IIbIIIA (GPIIbIIIa), for example in the treatment of immune thrombocytopenia purpura (ITP); and
proteinase 3 (PR3), for example in the treatment of the Wegener's disease.

In an embodiment, the conjugate of the invention comprises an anti-PF4 antibody or an anti-PF4 antibody fragment, for example the antibody 1E12 or a fragment of the antibody 1E12 [2].

### First Aspect of the Conjugate According to the Invention

According to a first aspect, the conjugate according to the invention is of formula (A):

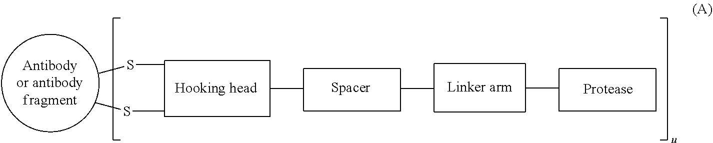

wherein u=1, 2, 3, 4, 5 or 6.

The number of units between hooks, represented by the letter u, varies according to the number of units bonded to the antibody or to the antibody fragment. The maximum number of units bonded to the antibody or to the antibody fragment will depend on the number of disulfide bridges of the antibody or antibody fragment.

In some embodiments:
the antibody is IgG1 or IgG4 and u=1, 2, 3 or 4, preferably u=4;
the antibody is IgG2 and u=1, 2, 3, 4, 5 or 6, preferably u=6;
the antibody fragment is Fab and u 1;
the antibody fragment is Fab′ and u=1 or 2, preferably u=2;
the antibody fragment is F(ab)₂ and u=1, 2, 3 or 4, preferably u=4.

The bond between each of the different portions of the compound of formula (A), namely hooking head, spacer, linker arm and protease, is achieved via an amide, ester, ether, carbamate or carbonate bond, or by implementing a so-called “click” reaction, as explained below.

The following embodiments can be combined with one another as applicable.

In an embodiment, the hooking head is a compound of formula (I):

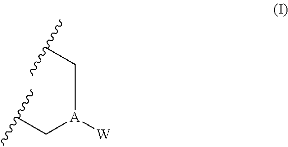

wherein:
   A is the residue of a phenyl or pyridyl;
   W is —ORₐ, —COR₂, —CONR₃R₄ or —NR₃COR₄;
      Rₐ is —(CH₂CH₂O)_{q}—(CH₂)ᵣ-R₅-, —(CHR_{c})ᵣ-R₅-, —COR_{b}, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣ-R₅- or —(CH₂CH₂O)_{q}—(CH₂)ᵣ—CONH—(CHR_{c})ᵣ-R₅-;
      R_{b} is —(CH₂CH₂O)_{q}—(CH₂)ᵣ-R₅-, —O(CH₂CH₂O)_{q}—(CH₂)ᵣ-R₅-, —(CHR_{c})ᵣ-R₅-, —O(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣ-R₅— or —(CH₂CH₂O)_{q}—(CH₂)ᵣ—CONH—(CHR_{c})ᵣ-R₅-;
      R₂ is —(CH₂CH₂O)_{q}—(CH₂)ᵣ-R₅-, —(CHR_{c})ᵣ-R₅-, —O(CH₂CH₂O)_{q}—(CH₂)ᵣ-R₅-, —O(CHR_{c})ᵣ-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)_{q}—(CH₂)ᵣ—NHCO—(CR_{c}H)ᵣ-R₅— or —O(CH₂CH₂O)_{q}—(CH₂)ᵣ—CONH—(CHR_{c})ᵣ-R₅-;
      R₃ is —H, —(C₁-C₆)alkyl or —(CH₂)ᵥ—SO₃H, preferably R₃ is —H or —(C₁-C₆)alkyl;
      R₄ is —(CH₂CH₂O)_{q}—(CH₂)ᵣ-R₅-, —(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣ-R₅— or —(CH₂CH₂O)_{q}—(CH₂)ᵣ—CONH—(CHR_{c})ᵣ-R₅-;
   each R₅ is chosen from:

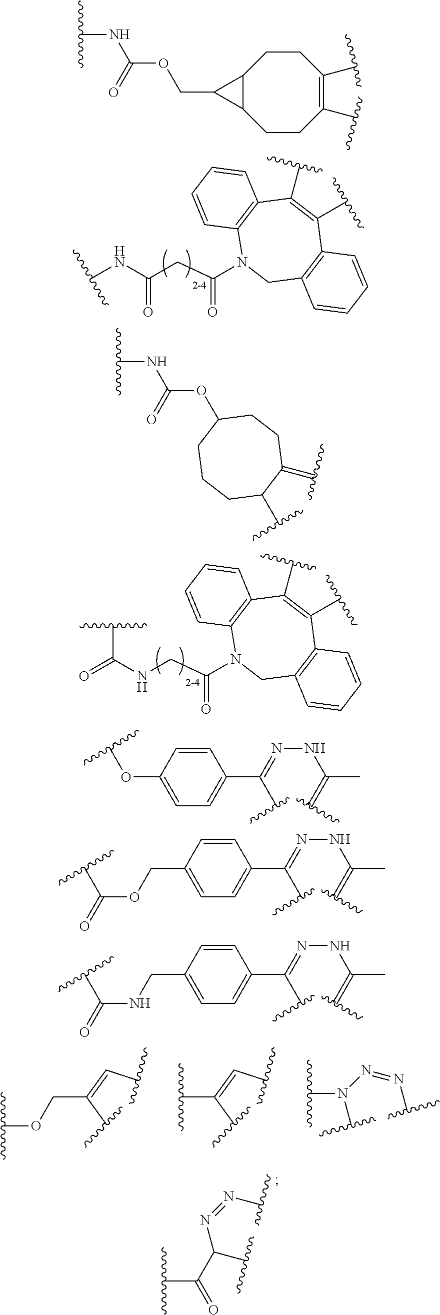

each R_{c} is chosen from —H, —CH₂SO₃H or —SO₃H;
q is an integer ranging from 1 to 24;
each r is an integer ranging from 1 to 8;
v is an integer ranging from 1 to 6.

In an embodiment, the spacer is a direct bond or a group of formula
(R₅-R₆-R₅′)ₛ- wherein:
R₅ is as defined above;
R₅′ is chosen from:

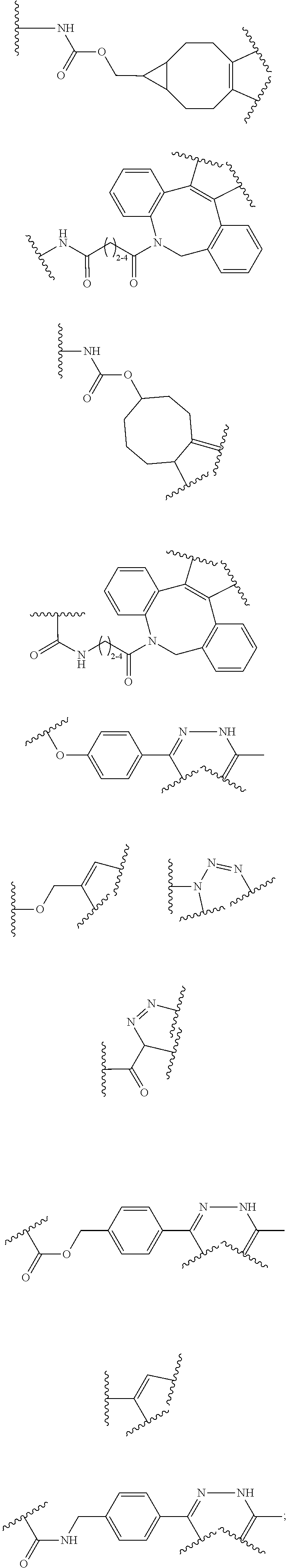

it being understood that R₅ and R′₅ cannot be identical;
   R₆ is —(CH₂CH₂O)_{q}—(CH₂)ᵣ—, —(CH₂CH₂O)_{q}—(CH₂)ᵣ—CO-R₇-NH—(CH₂CH₂O)_{q}—(CH₂)ᵣ—, —(CH₂CH₂O)_{q}—(CH₂)ᵣ—NH-R₈—CO—(CH₂CH₂O)_{q}—(CH₂)ᵣ—, —(CH₂)ᵣ—NHCO—(CHR_{c})ᵣ—, —(CH₂CH₂O)_{q}—(CH₂)ᵣ—CONH—(CH₂CH₂O)_{q}—(CH₂)ᵣ—NHCO—(CH₂CH₂O)_{q}—(CH₂)ᵣ—, —(CH₂CH₂O)_{q}—(CH₂)ᵣ—CONH—(CHR_{c})ᵣ—NHCO—(CH₂CH₂O)_{q}—(CH₂)ᵣ—, —(CH₂CH₂O)_{q}—(CH₂)ᵣ—NHCO—(CH₂CH₂O)_{q}—(CH₂)ᵣ—CONH—(CH₂CH₂O)_{q}—(CH₂)ᵣ— or —(CH₂CH₂O)_{q}—(CH₂)ᵣ—NHCO—(CHR_{c})ᵣ—CONH—(CH₂CH₂O)_{q}—(CH₂)ᵣ—;
   s is an integer ranging from 1 to 10;
      R₇ is a direct bond, —NH—(CH₂CH₂O)_{q}—(CH₂)ᵣ—CO— or —NH—(CHR_{c})ᵣ—CO—;
      R₈ is a direct bond, —CO—(CH₂CH₂O)_{q}—(CH₂)ᵣ—NH— or —CO—(CHR_{c})ᵣ—NH—;
      each R_{c} is chosen from —H, —CH₂—SO₃H or —SO₃H;
      each q is an integer ranging from 1 to 24;
      each r is an integer ranging from 1 to 8.

In an embodiment, the linker arm is —CO—(CH₂)ₜ-R₉—,

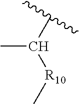

or —CH₂-R₁₀-;
t is an integer ranging from 1 to 10;
R₉ is -R₅-, —CO-R₇-NH—(CH₂CH₂O)_{q}—(CH₂)ᵣ-R₅-, —NH-R-CO—(CH₂)ᵣH₂O)_{q}—(CH₂)ᵣ-R₅-, —CO-R₇-NH—(CH₂)ᵣ-R₅-, —NH-R₈-CO—(CH₂)ᵣ-R₅-, —CONH—(CH₂CH₂O)_{q}—(CH₂)ᵣ—NHCO—(CH₂CH₂O)_{q}—(CH₂)ᵣ-R₅-; —CONH—(CHR_{c})ᵣ—NHCO—(CH₂CH₂O)_{q}—(CH₂)ᵣ-R₅-, —NHCO—(CH₂CH₂O)_{q}—(CH₂)ᵣ—CONH—(CH₂CH₂O)_{q}—(CH₂)ᵣ-R₅-; —NHCO—(CHR_{c})ᵣ—CONH—(CH₂CH₂O)_{q}—(CH₂)ᵣ-R₅-;
R₁₀ is a group of formula:

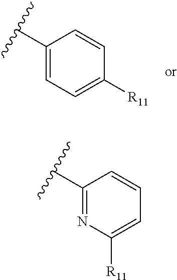

R₁₁ is —(CH₂)ᵣ-R₅-, —CONH—(CH₂)ᵣ-R₅-, —NHCO—(CH₂)ᵣ-R₅-, —NH—(CH₂)ᵣ-R₅-, —CONH—(CH₂CH₂O)_{q}—(CH₂)ᵣ-R₅-, —NHCO—(CH₂CH₂O)_{q}—(CH₂)ᵣ-R₅-;
R₅, R₂ and R₈ are as defined above;
each R_{c} is chosen from —H, —CH₂—SO₃H or —SO₃H;
each q is an integer ranging from 1 to 24;
each r is an integer ranging from 1 to 8.

In an embodiment, W is —CONR₃R₄;
R₃ is as previously defined, and v is an integer ranging from 1 to 6;
R₄ is —(CH₂CH₂O)_{q}—(CH₂)ᵣ-R₅-, or —(CHR_{c})ᵣ-R₅-;
   R₅ is chosen from:

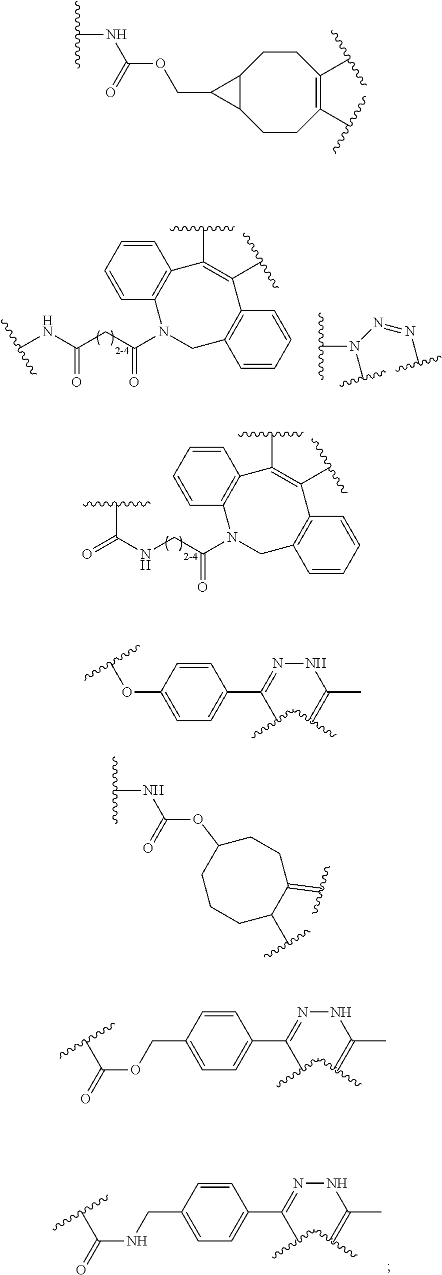

each R_{c} is chosen from —H, —CH₂—SO₃H or —SO₃H;
q is an integer ranging from 1 to 12;
r is an integer ranging from 1 to 6.

In this embodiment, R₄ and R₅ are advantageously defined as follows:
R₄ is —(CH₂CH₂O)_{q}—(CH₂)ᵣ-R₅-, or —(CHR_{c})ᵣ-R₅-;
   R₅ is chosen from:

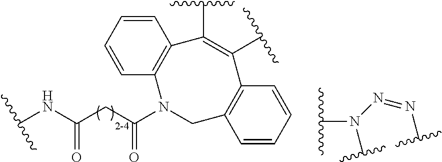

q is an integer ranging from 1 to 12;
r is an integer ranging from 1 to 6.

In an embodiment, the hooking head is a compound of formula (Ia):

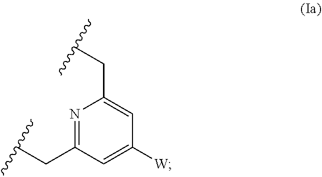

W is as defined above.

In a preferred embodiment, the hooking head is a compound of formula (Ib) or (Ib′):

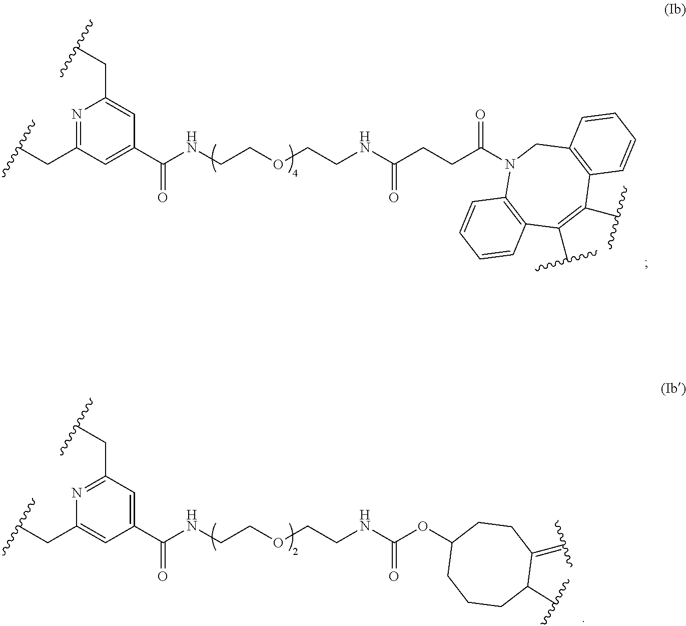

In a preferred embodiment, the portion constituted by the spacer and the linker arm is represented by formulas (III) or (IV):

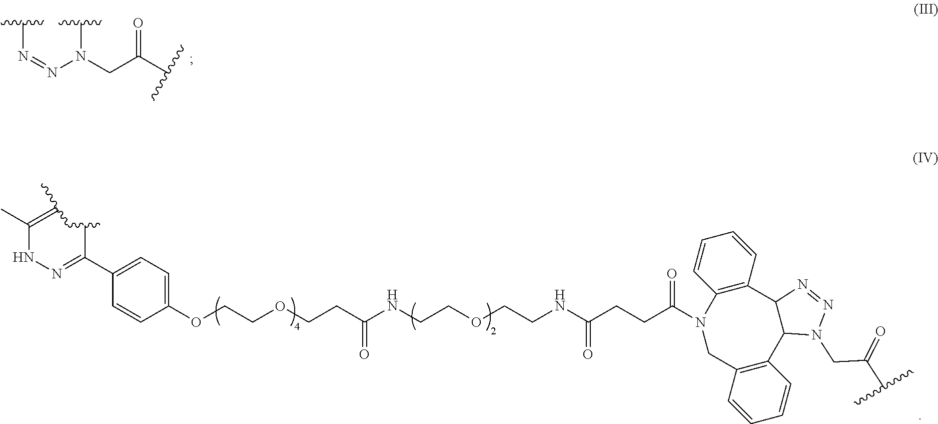

In an embodiment, the conjugate is of formula (V) or (VI):

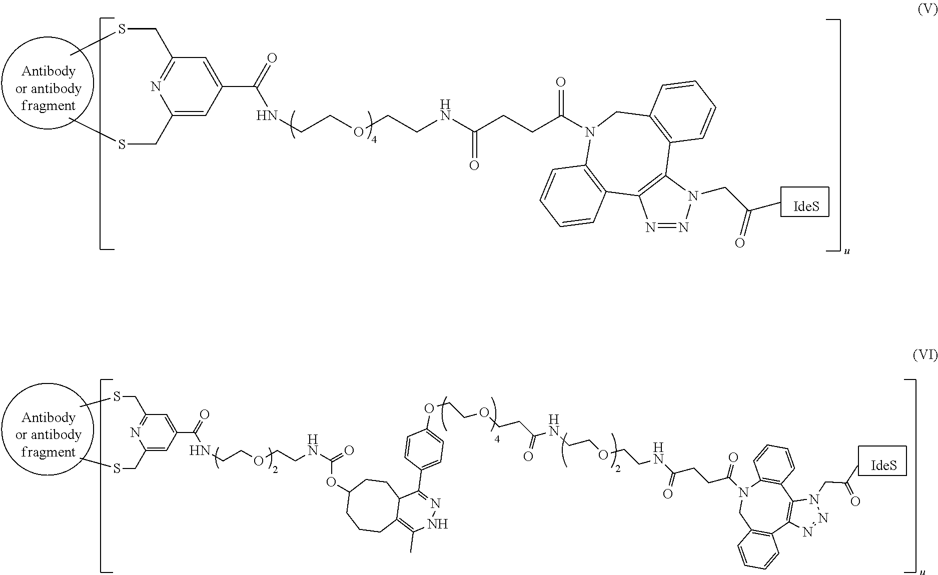

wherein u is as previously defined, preferably u=1, 2 or 4.

In an embodiment, the conjugate is of formula (V′) or (VI′):

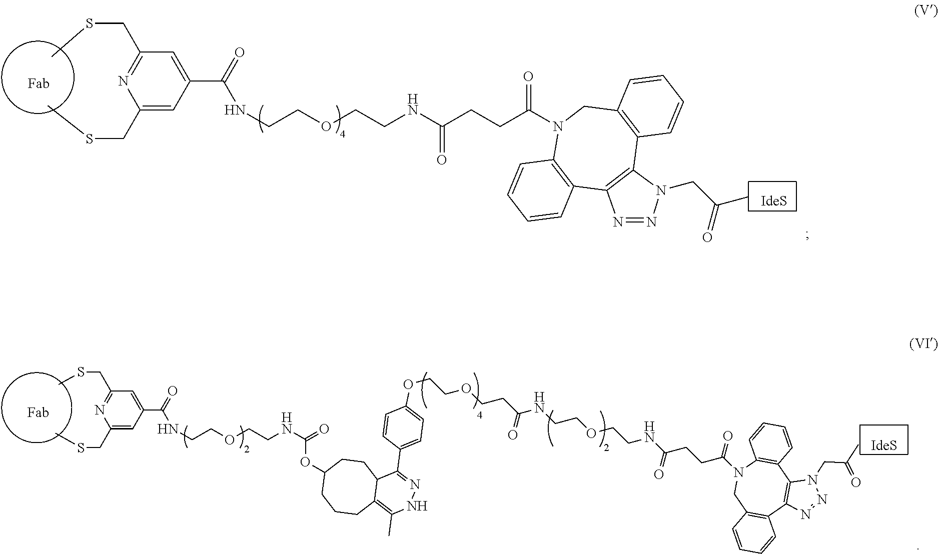

Compound 24 of the examples corresponds to a compound of formula (VI′), wherein Fab is the fragment Fab of the trastuzumab antibody.

### Second Aspect of the Conjugate According to the Invention

According to a second aspect, the conjugate according to the invention is of formula (B) or (C):

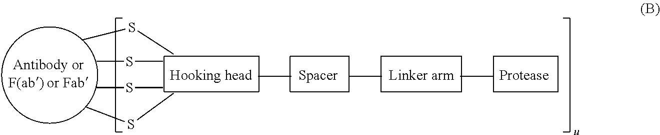

wherein u=1, 2 or 3.

The number of units between hooks, represented by the letter u, varies according to the number of units bonded to the antibody or to the antibody fragment. The maximum number of units bonded to the antibody or to the antibody fragment will depend on the number of disulfide bridges of the antibody or antibody fragment.

In some embodiments:
the antibody is IgG1 or IgG4 and u=1 or 2;
the antibody is IgG2 and u=1, 2 or 3, preferably u=1;
the antibody fragment is Fab′ and u=1;
the antibody fragment is F(ab)₂ and u=1 or 2.

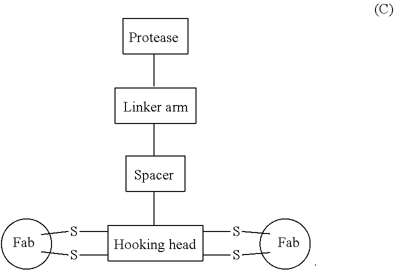

The following embodiments can be combined with one another as applicable.

In an embodiment, the hooking head is a compound of formula (II):

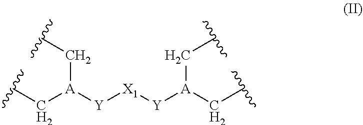

wherein:
   each A is the residue of a phenyl or a pyridyl;
   each Y is a direct bond, —CH₂—, —O—, —S—, —CO—, —NH— or —C(═NR₁)—, with R₁ which is chosen from —H, —OH or —(C₁-C₆)alkyl;
   X₁ is chosen from:

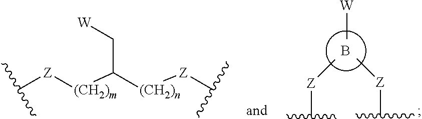

[symbol] is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxy]methyl]-5-prop-2-ynoxy-benzene;
   each Z is a direct bond, —CH₂—, —O—, —S—, —CO—, —NH— or —C(═NR₁)—;
   W is as defined for the conjugate of formula (A).

In an embodiment, the spacer is a direct bond or a group of formula -(R₅-R₆-R₅′ₛ wherein R₅, -R₅′ and R₆ are as defined for the conjugate of formula (A).

In an embodiment, the linker arm is —CO—(CH₂)ₜ-R₉-,

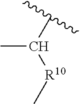

or —CH₂-R₁₀-, with:
t is an integer ranging from 1 to 10;
R₉ is -R₅-, —CO-R₂-NH—(CH₂CH₂O)_{q}—(CH₂)ᵣ-R₅-, —NH-Re-CO—(CH₂CH₂O)_{q}—(CH₂)ᵣ-R₅-, —CO-R₂-NH—(CH₂)ᵣ-R₅-, —NH-Re-CO—(CH₂)ᵣ-R₅-, —CONH—(CH₂CH₂O)_{q}—(CH₂)ᵣ—NHCO—(CH₂CH₂O)_{q}—(CH₂)-R₅-; —CONH—(CHR_{c})ᵣ—NHCO—(CH₂CH₂O)_{q}—(CH₂)-R₅-, —NHCO—(CH₂CH₂O)_{q}—(CH₂)ᵣ—CONH—(CH₂CH₂O)_{q}—(CH₂)-R₅; —NHCO—(CHR_{c})ᵣ—CONH—(CH₂CH₂O)_{q}—(CH₂)ᵣ-R₅-;
R₁₀ is a group of formula:

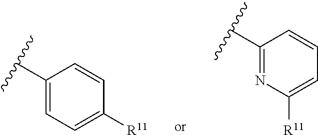

wherein
   R₁₁ is —(CH₂)ᵣ-R₅-, —CONH—(CH₂)ᵣ-R₅-, —NHCO—(CH₂)ᵣ-R₅-, —NH—(CH₂)ᵣ-R₅-, —CONH—(CH₂CH₂O)_{q}—(CH₂)ᵣ-R₅-, —NHCO—(CH₂CH₂O)_{q}—(CH₂)ᵣ-R₅-;
   R₂ is a direct bond, —NH—(CH₂CH₂O)_{q}—(CH₂)ᵣ—CO— or —NH—(CHR_{c})ᵣ—CO—;
   R₅ is a direct bond, —CO—(CH₂CH₂O)_{q}—(CH₂)ᵣ—NH— or —CO—(CHR_{c})ᵣ—NH—;
   R₅ is chosen from:

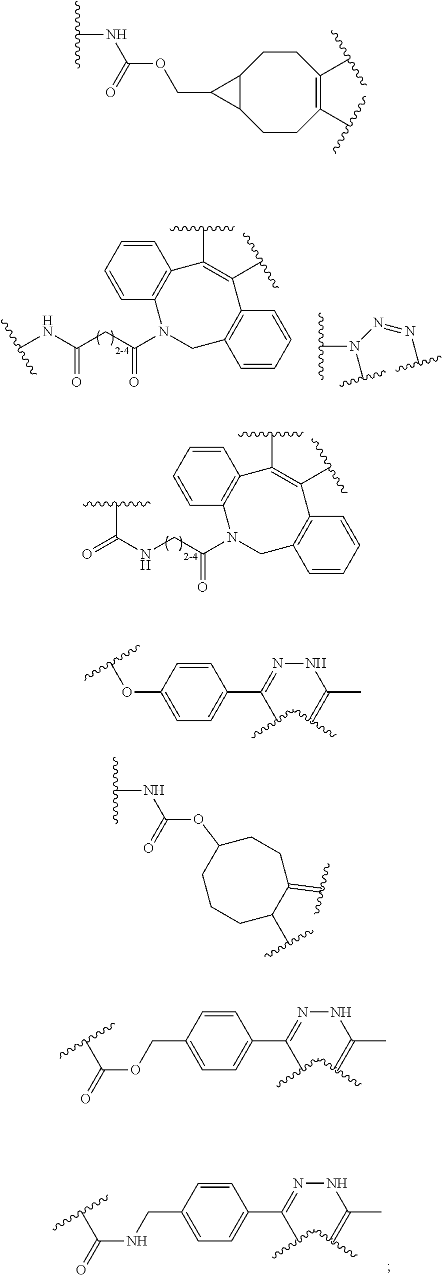

each R_{c} is chosen from —H, —CH₂—SO₃H or —SO₃H;
each q is an integer ranging from 1 to 24;
each r is an integer ranging from 1 to 8.

In an embodiment, each A is the residue of a pyridyl.

In an embodiment, each Y is chosen from a direct bond, —CO— and —NH—.

In an embodiment, one of the groups Y and Z is —CO— and the other is —NH—.

In an embodiment, X₁ is a group of formula:

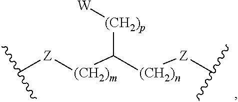

wherein
   Z is —CO— or —NH—;
   m and n are each, independently of one another, an integer ranging from 0 to 3;
   p is equal to 0, 1 or 2;
   W is —CONR₃R₄;
      R₄ is —(CH₂CH₂O)_{q}—(CH₂)ᵣ-R₅-, or —(CHR_{c})ᵣ-R₅-;
      R₅ is chosen from:

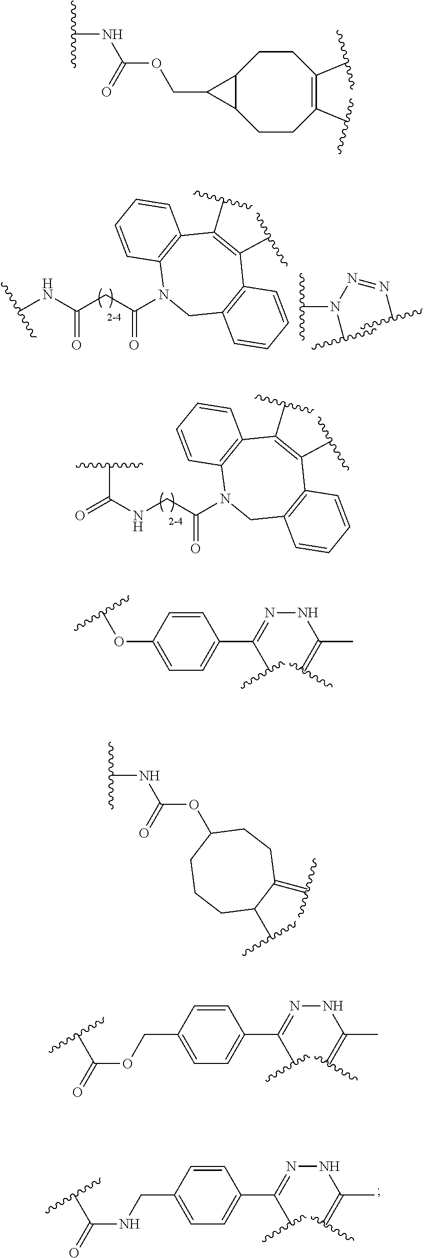

each R_{c} is chosen from —H, —CH₂—SO₃H or —SO₃H;
R₃ is —H, —(C₁-C₆)alkyl or —(CH₂)ᵥ—SO₃H, preferably R₃ is —H or —(C₁-C₆)alkyl;
q is an integer ranging from 1 to 12;
r is an integer ranging from 1 to 6;
v is an integer ranging from 1 to 6.

In a preferred embodiment, X₁ is a group:

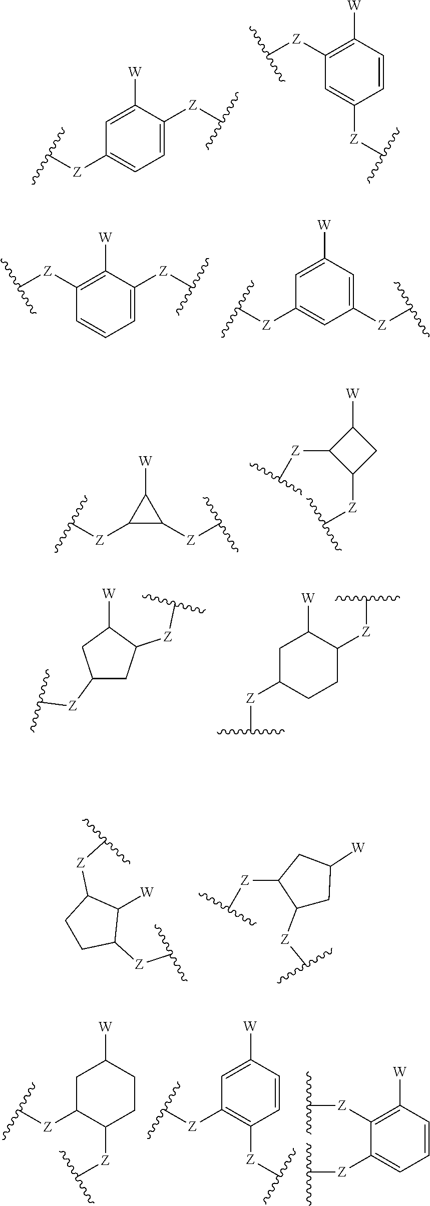

chosen from:

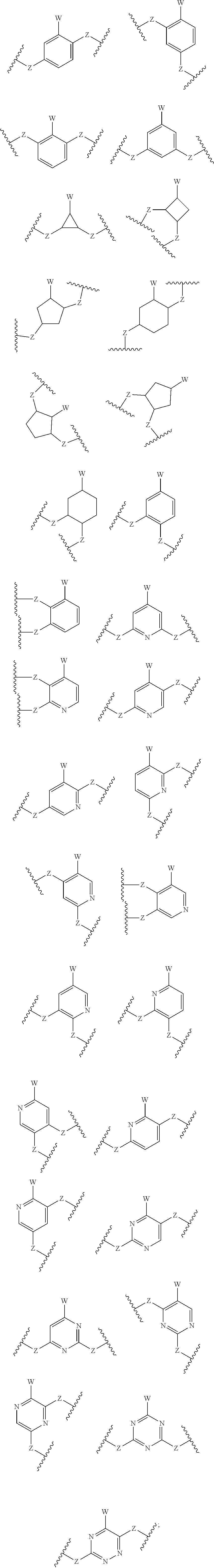

Z is —CO— or —NH—;
W is —CONR₃R₄;
   R₃ is —H or —(C₁-C₆)alkyl;
   R₄ is —(CHR_{c})ᵣ-R₅-, or —(CH₂CH₂O)_{q}—(CH₂)ᵣ-R₅-;
   R₅ is chosen from:

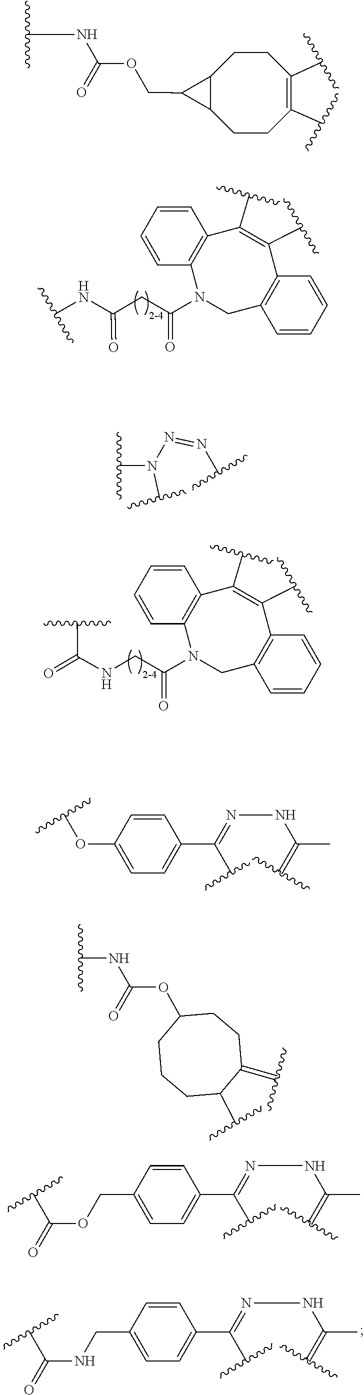

each R_{c} is chosen from —H, —CH₂—SO₃H or —SO₃H;
q is an integer ranging from 1 to 12;
r is an integer ranging from 1 to 6.

In a particularly preferred embodiment, X₁ is chosen from:

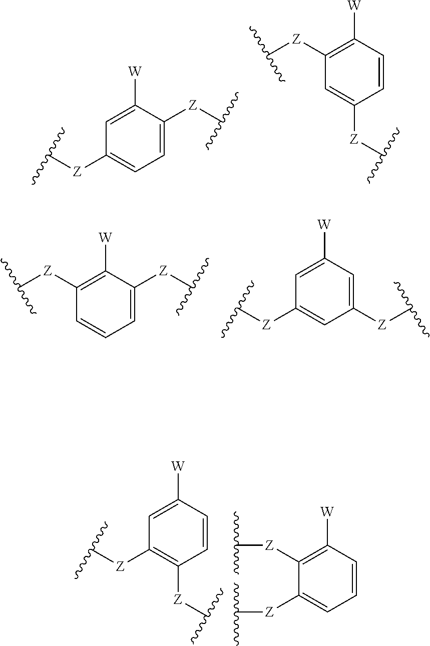

in each of these formulas W and Z are as previously defined.

In an embodiment, W is —CONR₃R₄;
R₃ is —H or —(C₁-C₆)alkyl;
R₄ is —(CH₂CH₂O)_{q}—(CH₂)ᵣ-R, or —(CHR_{c})ᵣ-R₅-;
   R₅ is chosen from:

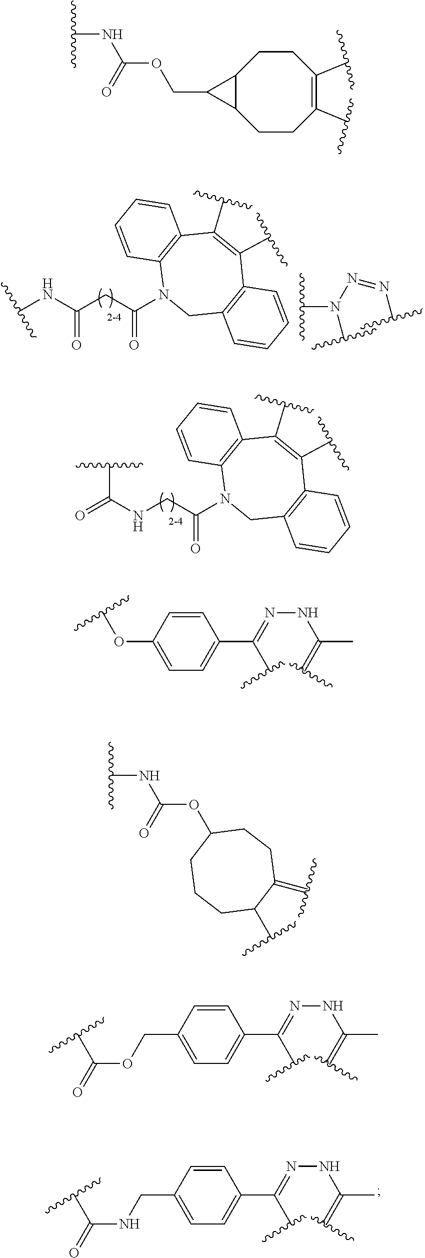

each R_{c} is chosen from —H, —CH₂—SO₃H or —SO₃H;
m and n are each, independently of one another, an integer ranging from 0 to 3;
p is equal to 0, 1 or 2;
q is an integer ranging from 1 to 12;
r is an integer ranging from 1 to 6.

In an embodiment, the hooking head is a compound of formula (IIa), (IIa′), (IIb), (IIb′), (IIc) or (IIc′):

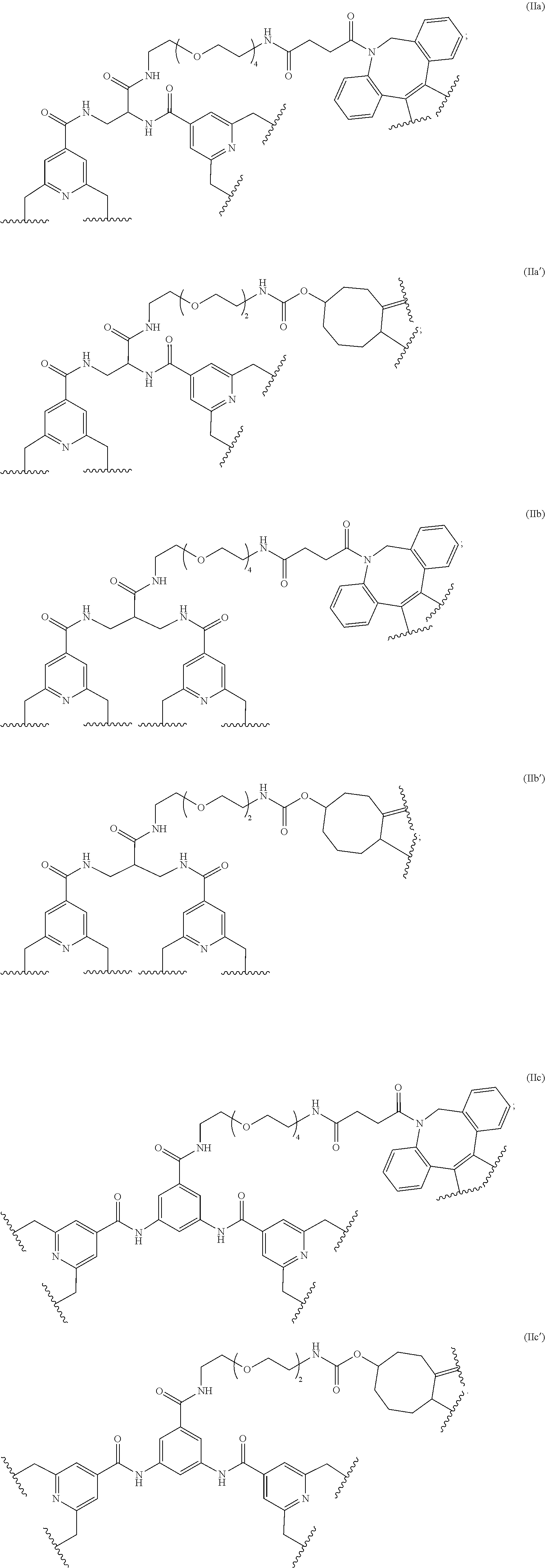

In an embodiment, the portion constituted by the spacer and the linker arm is represented by the formulas (III) or (IV):

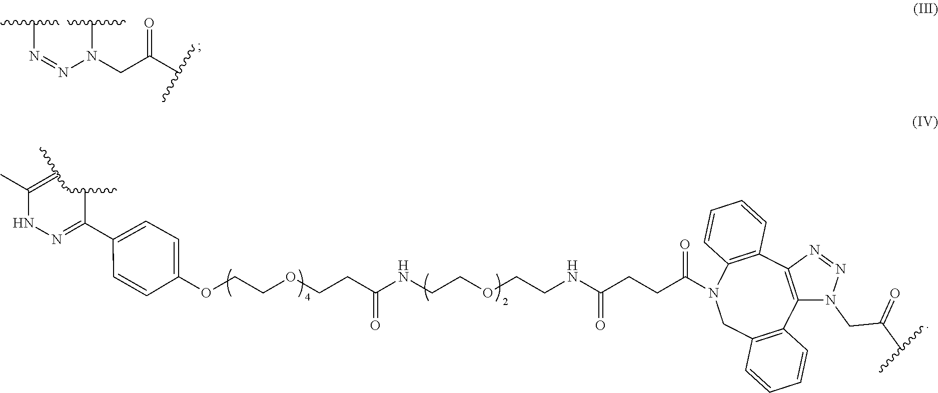

In an embodiment, the conjugate is of formula (VII) or (VIII):

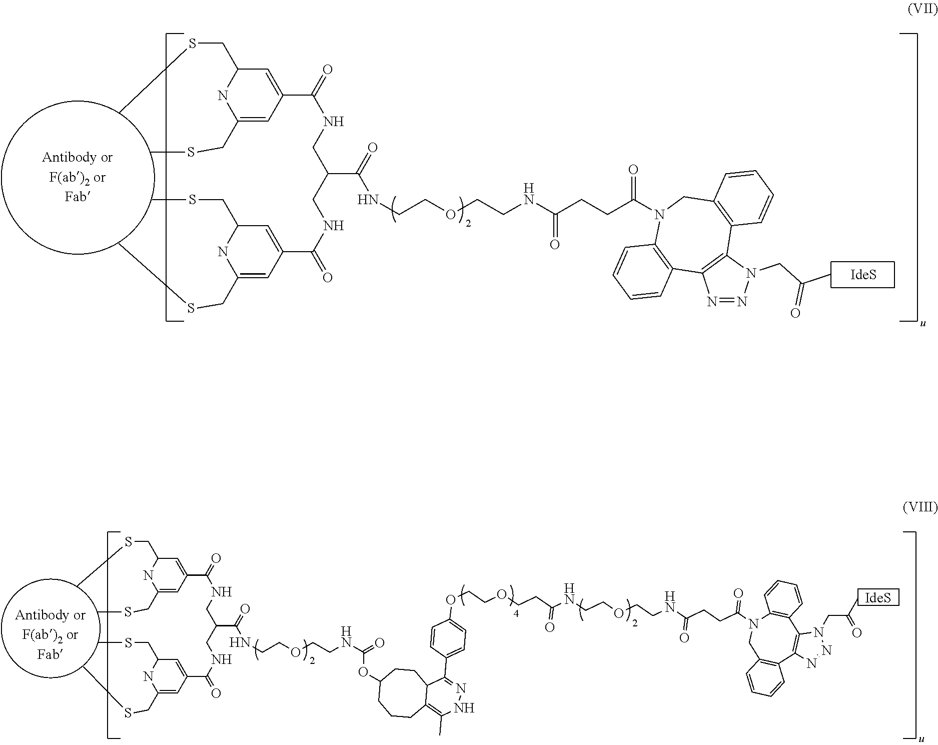

wherein u=1 or 2.

In an embodiment, the conjugate is of formula (VII′) or (VIIII′):

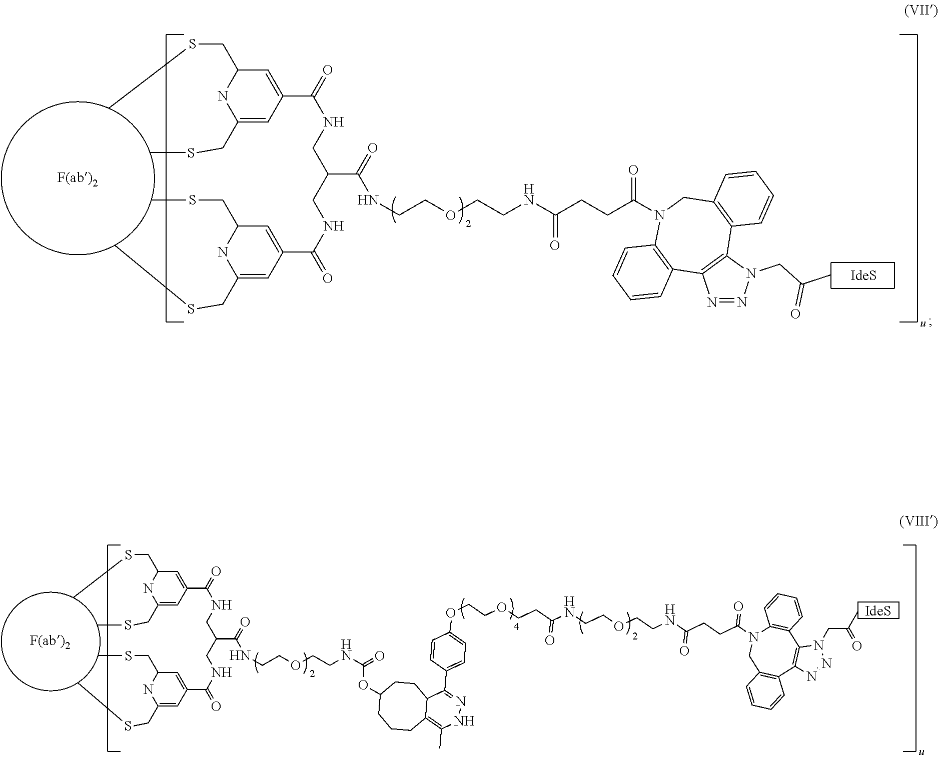

wherein u=1 or 2.

In an embodiment, the conjugate is of formula (IX) or (X):

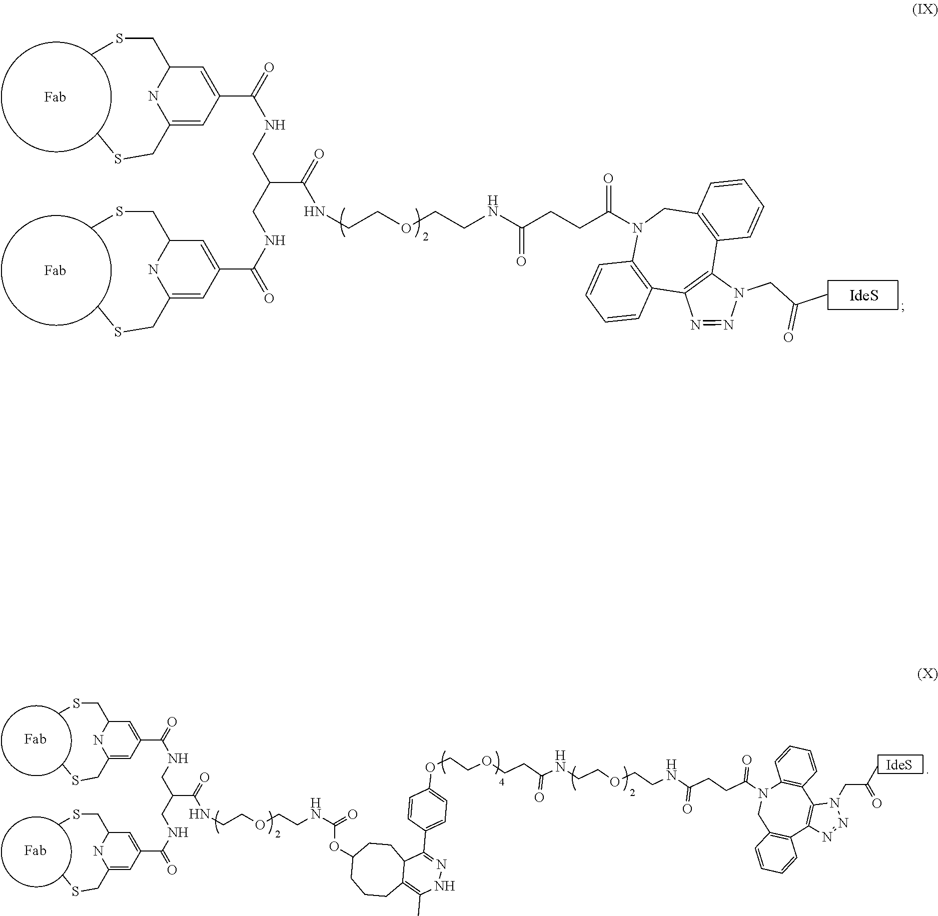

### Composition of Conjugates

In an embodiment, the conjugate according to the invention is “purified” or “isolated”.

The conjugate according to the invention can also be formulated in a composition.

Thus, a second object of the invention relates to a composition comprising one or more conjugates as defined above. The composition can be a pharmaceutical composition containing one or more excipients and/or pharmaceutically acceptable carriers.

### Therapeutic Indications

A third object of the invention relates to a conjugate according to the invention or a composition comprising one or more conjugates according to the invention, for use as drug.

A third object of the invention relates to a conjugate according to the invention or a composition comprising one or more conjugates according to the invention for use in the treatment of a disease induced by autoantibodies.

In an embodiment, the autoantibodies are IgG. Thus, the disease induced by autoantibodies can be a disease induced by IgG, such as an autoimmune disease induced by IgG. More specifically, the conjugate according to the invention can target the specific protease of the hinge region of the IgG at the site of action of the pathogenic IgG which induce the autoimmune disease. The conjugate thus targeted will therefore treat the disease by cleaving the hinge region of the pathogenic IgG, which can inactivate them.

In an embodiment, the autoimmune disease induced by the IgG is chosen from Biermer's anaemia, autoimmune haemolytic anaemia, primary sclerosing cholangitis, type I diabetes, epidermolysis bullosa, (Hashimoto's) hypothyroiditsm, systemic lupus erythematosus, coeliac disease, Basedow's hypothyroidism, Crohn's disease, myasthenia gravis, bullous pemphigoid, deep pemphigus, polymyositis, immune thrombocytopenic purpura, haemorrhagic rectocolitis, stiff-man syndrome, Lambert-Eaton myasthenic syndrome, Goujerot-Sjogren syndrome, Guillain-Barré syndrome, multiple sclerosis, psoriasis, rheumatoid polyarthritis, anti-phospholipid antibody syndrome, Horton disease, acute disseminated encephalomyelitis, glomerulonephritis, Goodpasture syndrome, Wegener's disease, Churg-Strauss syndrome, Morvan's syndrome, systemic scleroderma, vitiligo, Behcet's disease, heparin-induced thrombocytopenias, thrombotic thrombocytopenia induced by a respiratory virus, vaccine-induced thrombotic thrombocytopenia.

Advantageously, the autoimmune disease is chosen from immune thrombocytopenic purpura (ITP), antiphospholipid syndrome, autoimmune haemolytic anaemia or Wegener's disease, drug-induced thrombocytopenias, such as heparin-induced thrombocytopenias.

Another object of the invention relates to a conjugate according to the invention or a composition comprising one or more conjugates according to the invention for use in the treatment of a thrombotic thrombocytopenia induced by a respiratory virus, such as a coronavirus, preferably SARS-CoV-2.

Another object of the invention relates to a conjugate according to the invention or a composition comprising one or more conjugates according to the invention for use in the treatment of a vaccine-induced thrombotic thrombocytopenia (VITT), such as a vaccine against a coronavirus, preferably SARS-CoV-2.

The conjugate or the composition according to the invention is preferably formulated for a parenteral administration, for example an intravascular administration (intravenous or intra-arterial), intraperitoneal or intramuscular. The term “administered parenterally”, as used here, designates administration routes other than enteral and topical administration, generally by injection, and comprises, without limitation, intravascular, intravenous, intramuscular, intra-arterial, intrathecal, intracapsular, intra-orbital, intracardiac, intradermic and intraperitoneal administration, by injection, transtracheal, subcutaneous, intra-articular, subcapsular, subarachnoidal, intraspinal and intrasternal infusion. Intravenous administration is preferred in the context of the present invention, for example by intravenous infusion.

The dose of conjugate administered to the subject who has need of it will vary according to several factors, including its limitation, the administration route, type and severity of the pathology treated, condition of the patient, corpulence of the patient, age of the patient, etc. A person skilled in the art can easily determine, on the basis of his knowledge in this field, the dosage range required based on these and other factors. The appropriate dose can also be determined using animal models or with clinical trials. The administration can be performed once or, more generally, several times. The administration scheme may comprise an initial low dose then maintenance doses, for example weekly, once every two weeks, once every three weeks, every month, or more. The duration of treatment can vary according to the pathology treated on the subject.

The conjugate or the composition according to the invention can be used in monotherapy or in combination with drugs for which the therapeutic interest is recognised for the pathology considered.

### Method for Preparing Conjugates

### Conjugates of Formula (A)

Conjugates of formula (A) can be prepared by reaction between a compound of formula (a₁) or (a₂):

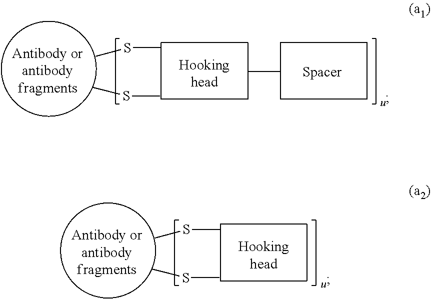

and a compound of formula (a₃):

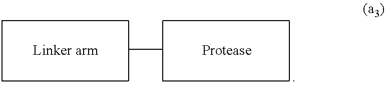

In the formulas (a₁) and (a₂), u is as defined for the conjugate of formula (A).

The compound of formula (a₂) can be prepared by conjugation of an antibody or an antibody fragment with a compound of formula (I_{bls}):

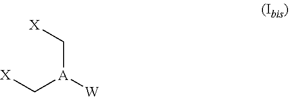

wherein X is a halogen, preferably Br, and A and W are as previously defined.

The compound of formula (a₁) is prepared by reaction of the compound of formula (a₂) with a spacer.

The conjugates of formula (A) can also be prepared by reaction between a compound of formula (a₂) as previously defined and a compound of formula (a₄):

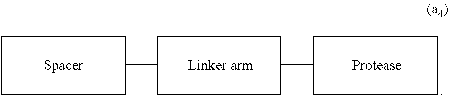

### Conjugates of Formula (B)

Conjugates of formula (B) can be prepared by reaction between a compound of formula (b₁) or (b₂):

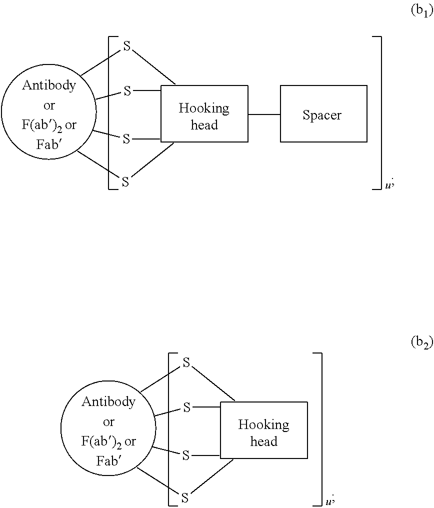

and a compound of formula (as) as previously defined. In formulas (b₁) and (b₂), u is as defined for the conjugate of formula (B).

The compound of formula (b₂) can be prepared by conjugation of an antibody or of an F(ab′)₂ or an Fab′ with a compound of formula (II_{bls}):

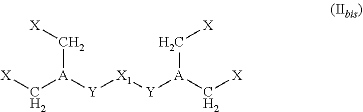

wherein X is a halogen, preferably Br, and A, Y and X₁ are as previously defined.

The compound of formula (b₁) is prepared by reaction between a compound of formula (b₂) and a spacer.

Conjugates of formula (B) can also be prepared by reaction between a compound of formula (b₂) and a compound of formula (a₄) as previously defined.

### Conjugates of Formula (C)

Conjugates of formula (C) can be prepared by reaction between a compound of formula (c₁) or (c₂):

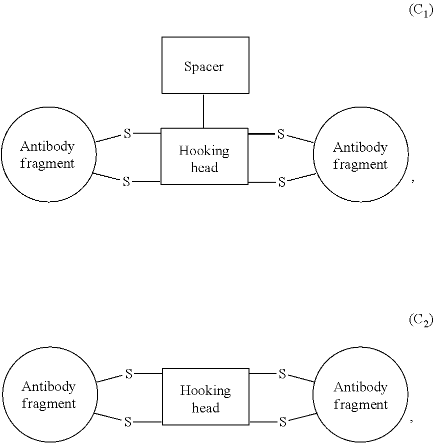

and a compound of formula (a₃) as previously defined.

The compound of formula (C₂) can be prepared by conjugation of two antibody fragments with a compound of formula (II_{bls}) as previously defined.

The compound of formula (c₁) is prepared by reaction of the compound of formula (c₂) with a spacer.

The conjugates of formula (C) can also be prepared by reaction between a compound of formula (c₂) and a compound of formula (a₄), as previously defined.

In the reactions described above, the antibody, the antibody fragment, F(ab)₂, Fab′, the hooking head, the spacer (which may or may not be present), the linker arm and the protease are as previously defined, with the immediately following exceptions.

1/ When a compound comprises a hooking head that is not bonded to a spacer or to a linker arm (compounds (a₂), (b₂), (c₂)), and when the hooking head comprises a substituent R₅, this latter responds to one of the following formulas:

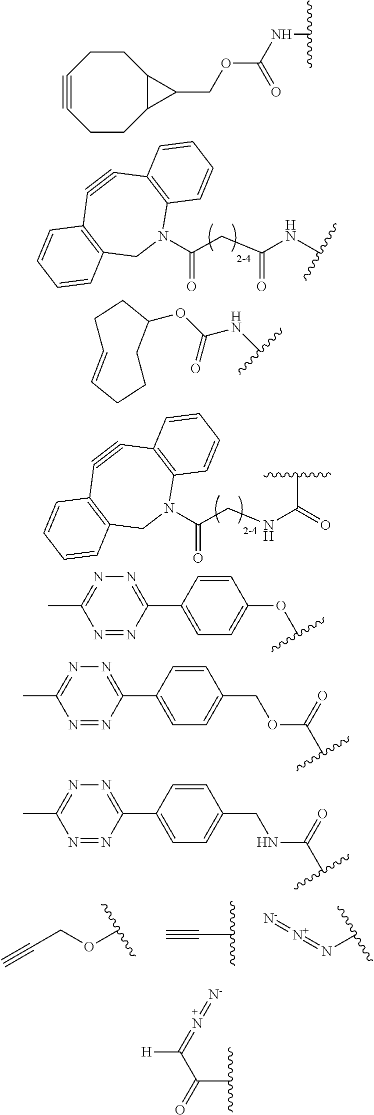

(the structures above have a single hooking point as opposed to three in the given definition of R₅ for conjugates (A), (B) and (C)—reference to R₅ here is an abuse of language justified in the present case for the sake of brevity).

2/ When the spacer is bonded to the hooking head (compounds (a₁), (b₁), (c₁)), then R₅ is as defined for conjugates (A), (B) and (C) and R′ₛ responds to one of the following formulas:

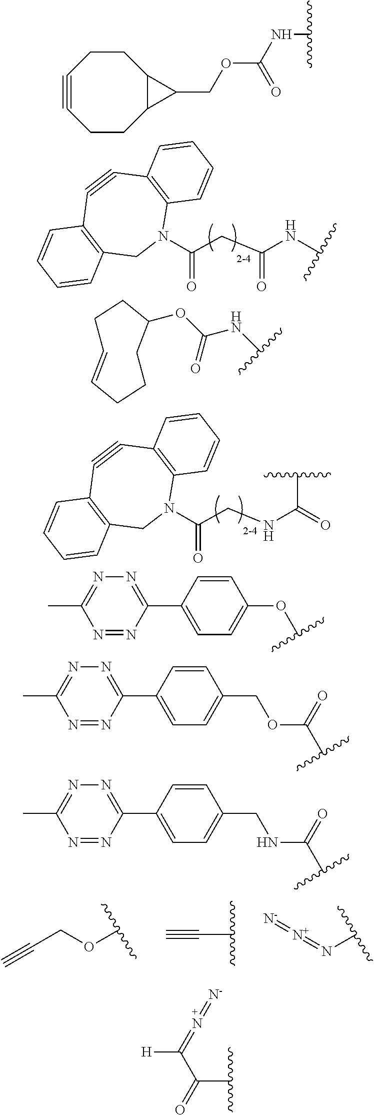

(the structures above have a single hooking point as opposed to three in the given definition of R′ₛ for conjugates (A), (B) and (C)—as before, reference to R′ₛ here is an abuse of language justified in the present case for the sake of brevity).

The preparation of compounds (a₁), (b₁), (c₁) comprises the reaction between the hooking head and the spacer. These two elements react with one another by implementing a so-called “click” reaction. More precisely, the click reaction between the hooking head and the spacer takes place between a diene (for example an azide or a diazo) and a dienophile (for example an alkene or an alkyne), each of these functions being provided by the R₅ group of the hooking head and of the spacer. Thus, the click reaction can be carried out between a diene carried by the R₈ group of the hooking head and a dienophile carried by the R₅ group of the spacer, or even between a dienophile carried by the R₅ group of the hooking head and a diene carried by the R₅ group of the spacer.

The preparation of compound (a₃) is carried out in a conventional manner, for example peptide bonding, between the protease and the linker arm.

The preparation of compound (a₄) can be carried out by reaction between a compound of formula (as) and a spacer, by means of a click reaction. This will be implemented between a diene carried by the R₅ group of the spacer and a dienophile carried by the R₅ group of the linker arm, or even between a dienophile carried by the R₅ group of the spacer and a diene carried by the R₅ group of the linker arm.

Compound (a₄) can also be prepared by first reacting the spacer and the linker arm by click reaction, as described above, then secondly reacting, in a conventional manner, the compound thus obtained with the protease (for example by peptide bonding between a CO group of the linker arm and an NH group of the protease).

The reaction between compound (a₁) or (a₂) and compound (a₃) is carried out by click reaction, between an R₅ group (diene or dienophile) carried by the spacer of compound (a₁) or the hooking head of compound (a₂), and an R₅ group (dienophile or diene) carried by the linker arm of compound (a₃).

The reaction between compound (b₁) or (b₂) and compound (a₃) is carried out by click reaction, between an R₅ group (diene or dienophile) carried by the spacer of compound (b₁) or the hooking head of compound (b₂), and an R₅ group (dienophile or diene) carried by the linker arm of compound (a₃).

The reaction between compound (c₁) or (c₂) and compound (c₃) is carried out by click reaction, between an R₅ group (diene or dienophile) carried by the spacer of compound (c₁) or the hooking head of compound (c₂), and an R₅ group (dienophile or diene) carried by the linker arm of compound (c₃).

The reaction between compound (a₂) and compound (a₄) is carried out by click reaction, between an R₅ group (diene or dienophile) carried by the hooking head of compound (a₂), and an R₅ group (dienophile or diene) carried by the spacer of compound (a₄).

The reaction between compound (b₂) and compound (a₄) is carried out by click reaction, between an R₅ group (diene or dienophile) carried by the hooking head of compound (b₂), and an R₅ group (dienophile or diene) carried by the spacer of compound (a₄).

The reaction between compound (c₂) and compound (a₄) is carried out by click reaction, between an R₅ group (diene or dienophile) carried by the hooking head of compound (c₂), and an R₈ group (dienophile or diene) carried by the spacer of compound (a₄).

Click reactions are well-known to a person skilled in the art and include, for example, a cycloaddition reaction between a dienophile and a diene. Examples of click reactions are shown in the following diagram:

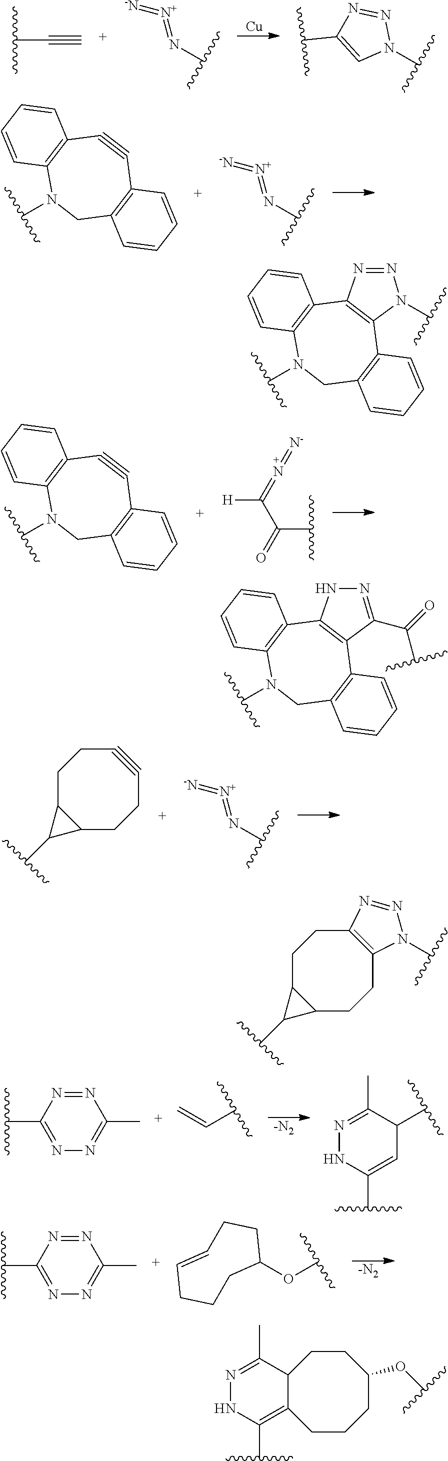

In these examples, a single regio-isomer by reaction has been shown, it being understood that cycloaddition reactions can generate a plurality of regio-isomers.

In an embodiment, the click reaction is therefore carried out between compounds of the following formulas:

TABLE 1

| CLICK PARTNER 1 | CLICK PARTNER 2 |
|---|---|
| Formula (a₁) | Formula (a₃) |
| Formula (a₂) | Formula (a₃) |
| Formula (a₂) | Formula (a₄) |
| Formula (b₁) | Formula (a₃) |
| Formula (b₂) | Formula (a₃) |
| Formula (b₂) | Formula (a₄) |
| Formula (c₁) | Formula (a₃) |
| Formula (c₂) | Formula (a₃) |
| Formula (c₂) | Formula (a₄) |

In this embodiment, the antibody or the antibody fragment bonds to the hooking head by substitution of the leaving groups represented by the substituent X in the formula (I_{bls}) or formula (II_{bls}). This results in the reconstruction of the antibody or antibody fragment, after reducing the interchain disulfide bridges. For example, in the context of the present invention, the reconstruction of a whole antibody is defined as obtaining a majority of a whole LHHL antibody. The proportion of of whole LHHL antibody and of other species (LHH, HH, LH, H, L) is determined using the optical density measured by SDS-PAGE gel analysis under denaturing reducing conditions. For example, a good reconstruction of a whole antibody is obtained when the proportion of LHHL exceeds 50%.

In an embodiment, implementing the methods described above makes it possible to obtain a composition with a ratio of protease per antibody/antibody fragment in the range from approximately 0.50 to approximately 5.0. In an embodiment, the antibody is conjugated on average to 4.00±1.00 (i.e. any value ranging from 3.00 to 5.00, for example 3.00; 3.01; . . . ; 4.99; 5.00) molecule(s), preferably to 4.00±0.50 proteases. In an embodiment, the antibody is conjugated on average to 2.00±0.50 (i.e. any value ranging from 1.50 to 2.50, for example 1.50; 1.51; . . . ; 2.49; 2.50) molecule(s), preferably to 2.00±0.30 protease(s). In an embodiment, the antibody is conjugated on average to 1.00±0.50 (i.e. any value ranging from 0.50 to 1.50, for example 0.50; 0.51; . . . ; 1.49; 1.50) molecule(s), preferably to 1.00±0.30 protease(s). In an embodiment, the Fab antibody fragment is conjugated on average to 1.00±0.30 (i.e. any value ranging from 0.70 to 1.30, for example 0.70; 0.71; . . . ; 1.29; 1.30) molecule(s), preferably to 1.00±0.10 protease.

In an embodiment, the Fab′ antibody fragment is conjugated on average to 2.00±0.50 (i.e. any value ranging from 1.50 to 2.50, for example 1.50; 1.51; . . . ; 2.49; 2.50) molecules, preferably to 2.00±0.30 proteases. In an embodiment, the Fab′ antibody fragment is conjugated on average to 1.00±0.50 (i.e. any value ranging from 0.50 to 1.50, for example 0.50; 0.51; . . . ; 1.49; 1.50) molecules, preferably to 1.00±0.30 proteases.

In an embodiment, the F(ab′)₂ antibody fragment is conjugated on average to 2.00±0.50 (i.e. any value ranging from 1.50 to 2.50, for example 1.50; 1.51; . . . ; 2.49; 2.50) molecule(s), preferably to 2.00±0.30 proteases. In an embodiment, the F(ab′)₂ antibody fragment is conjugated on average to 1.00±0.50 (i.e. any value ranging from 0.50 to 1.50, for example 0.50; 0.51; . . . ; 1.49; 1.50) molecule(s), preferably to 1.00±0.30 proteases. In an embodiment, the F(ab′)₂ antibody fragment is conjugated on average to 4.00±1.00 (i.e. any value ranging from 3.00 to 5.00, for example 3.00; 3.01; . . . ; 4.99; 5.00) molecules, preferably to 4.00±0.50 proteases.

The compounds of formulas (A), (B), (C), (a₁), (a₂), (b₁), (b₂), (c₁) and (c₂) can be prepared according to the techniques described in the literature, for example in the presence of a reducing agent. In an embodiment the antibody or the antibody fragment is in solution in a buffer. In an embodiment, the reducing agent is added before the compound to be conjugated on the antibody or antibody fragment. In another embodiment, the reducing agent and the compound to be conjugated on the antibody or the antibody fragment are added simultaneously.

The invention is illustrated by the examples below, given by way of illustration only. In these examples, the following abbreviations are used:
ADC=antibody-drug conjugate
BCN=bicyclononyne
HPLC=high-performance liquid chromatography
DBCO=dibenzylcyclooctyne
DCM=dichloromethane
DIPEA=N,N-diisopropylethylamine
DMF=N,N-dimethylformamide
DMSO=dimethyl sulfoxide
EDTA=ethylenediaminetetraacetic acid
EEDQ=N-ethoxycarbonyl-2-ethoxy-1.2-dihydroquinoline
eq=equivalent
HATU=hexafluorophosphate azabenzotriazole tetramethyl uronium
m/m=mass over mass ratio
m/z=mass over charge ratio
MAR=molecule-to-antibody ratio
ACN=acetonitrile
MeOH=methanol
MeOD=deuterated methanol
MPR=molecule-to-protease ratio
MgSO₄=magnesium sulfate
NaCl=sodium chloride
PEG=polyethylene glycol
PBS=phosphate-buffered saline
Pd/C=palladium over carbon
NMR=nuclear magnetic resonance
HRMS=high resolution mass spectrometry
AT=ambient temperature (20° C. unless indicated otherwise)
TCO=trans-cyclooctene
TFA=trifluoroacetic acid
THF=tetrahydrofurane
t_{R}=retention time
v/v=volume over volume ratio

### Analysis Methods

### Mass Spectrometry

Analyses by mass spectrometry (MS) were carried out using a Vion IMS Qtof mass spectrometer coupled to a Waters Acquity UPLC H-Class system (Wilmslow, UK) or a Bruker maXis mass spectrometer coupled with a Dionex Ultimate 3000 RSLC system.

On the Vion IMS Qtof mass spectrometer, before the MS analysis, the samples (20 μg) were desalted on a BEH SEC 2.1×150 mm 300 Å desalting column by an isocratic gradient (50 mM ammonium acetate, pH 6.5) at 40 μL/min. A bypass valve was programmed to allow the solvent to enter into the spectrometer between 6.5 and 9.5 minutes only. The MS data were acquired in positive mode with an ESI source over a range of 500 to 8000 m/z at 1 Hz and analysed using the UNIFI 1.9 software and the MaxEnt algorithm for the deconvolution.

On the Bruker maXis mass spectrometer, before the MS analysis, the samples (5 μg) were desalted on a MassPREP desalting column (2.1×10 mm, Waters), heated to 80° C. using a 0.1% aqueous solution of formic acid as solvent A and a 0.1% formic acid solution in ACN as solvent B at 500 μL/min. After 1 minute, a linear gradient of 5 to 90% of B in 1.5 minutes was applied. The MS data were acquired in positive mode with an ESI source over a range of 900 to 5000 m/z at 1 Hz and analysed using the DataAnalysis 4.4 software (Bruker) and the MaxEnt algorithm for the deconvolution. The number of:
linker arms or linker arm-spacers per protease (MPR),
hooking head or hooking head-spacer per antibody or antibody fragment (MAR),
proteases per antibody or antibody fragment,
   was determined using the intensity of the observed peaks.
   Peptide Coupling with EEDQ

Under an inert atmosphere, the acid was dissolved in anhydrous ACN, then EEDQ was added protected from light. The medium was stirred at 25° C., protected from light. A solution of free amine in the form of a TFA salt in anhydrous DMF in the presence of anhydrous DIPEA was added, protected from light. The medium was stirred at 25° C. until this reaction was completed. The reaction crude was purified by semi-preparative HPLC (Gilson PLC 2050 [ARMEN V2 system (pump) and ECOM TOYDAD600 (UV detector)] UV detection at 254 nm at 25° C.; Waters XBridge™ C-18 column; 5 μm (250 mm×19.00 mm); elution performed with 0.1% TFA (by volume) in water (solvent A), and acetonitrile (solvent B); gradient 20 to 100% of B for 37 minutes, then 100% B for 6 minutes at 17.1 mL/min) and the coupling product was obtained after concentration and lyophilisation.

### Bioconjugation Reactions

Bioconjugation buffer: 1× saline buffer, for example phosphate, borate, acetate, glycine, tris(hydroxymethyl)aminomethane, 4-(2-hydroxyethyl)-1-piperazine-ethanesulfonic acid in the pH range between 6 and 9, with a final NaCl concentration between 50 and 300 mM and a final concentration of EDTA between 0.1 and 10 mM. For example, 1× phosphate buffer at a pH of 8.3, with a final concentration of NaCl of 180 mM and a final concentration of EDTA of 1 mM.

Reducing agent: solution of a reducing agent chosen from dithiothreitol, 3-mercaptoethanol, tris(2-carboxyethyl)phosphine hydrochloride, tris(hydroxypropyl)phosphine at a concentration between 0.1 and 10 mM in the bioconjugation solution. For example, a 1 mM solution of tris(2-carboxyethyl)phosphine hydrochloride in the bioconjugation buffer.

Compounds: solutions of compounds 3, 4, 6 and 7 at a concentration between 0.1 and 10 mM in a mixture of organic solvents chosen from DMSO, DMF, MeOH, THF, ACN, N,N-dimethylacetamide, dioxane. For example, a 1 mM solution in a mixture of organic solvents composed of 20% DMF and 80% MeOH.

Purification: the reaction mixture was purified by steric exclusion on Sephadex® G-25 or PD-10 with buffer PBS Gibco™ 1× pH 7.4 in order to remove the residual chemical reagents.

If necessary, the purified protein was concentrated using a 10 kDa Amicon Ultra centrifugal concentrator with centrifuge (10,000 G) at +4° C.

### Functionalisation of Proteases

Functionalisation buffer: saline buffer, for example phosphate, borate, acetate, glycine, tris(hydroxymethyl)aminomethane, 4-(2-hydroxyethyl)-1-piperazineethanesulfonic acid at a concentration between 2 mM and 2 M in a pH range between 6 and 9, and with a final concentration of NaCl between 0 and 3 M. For example, HEPES 200 mM buffer at a pH of 7.5.

Solution of compounds 16 or 18 at a concentration between 1 and 1000 mM in an organic solvent (mixture or single solvent) chosen from DMSO, DMF, MeOH, THF, ACN, N,N-dimethylacetamide, dioxane. For example, a 100 mM solution in acetonitrile.

Purification: the reaction mixture was purified by steric exclusion on PD-10 (Cytiva) the buffer PBS Gibco™ 1× pH 7.4 in order to remove the residual chemical reagents.

SDS-PAGE analysis Tris-HCl SDS-PAGE gels were prepared with a concentration of 4% acrylamide on the concentration portion and a concentration of 6% acrylamide for the migration portion.

Laemmli 4× buffer (0.3 mM bromophenol blue; 2M glycerol, 20 mM TrisBase; 0.04% sodium dodecylsulfate) was added to the samples (1.2 μg), then the samples were incubated at 95° C. for 10 minutes. A large amplitude molecular weight marker (Invitrogen SeeBlue® Plus2 Prestained Standard) was used to estimate the molecular weight of the proteins. The gel was set to migrate at 100 V for 10 minutes then at 140 V for 35 minutes, in a NuPAGE migration buffer (50 mM MOPS; 50 mM TrisBase; 0.1% SDS (v/v); 1 mM EDTA, pH 7.3). After washing with water, the gel was stained with Coomassie blue (Thermo Scientific Imperial™ Protein Stain). The densitometric analysis was performed using the ImageJ software and a Windows Vanilla filter was applied for the analysis in black and white.

### EXAMPLES

### Example 1: DBCO 2,6-bis(bromomethyl)isonicotamido-PEG₂-ethyl-carbamate (3)

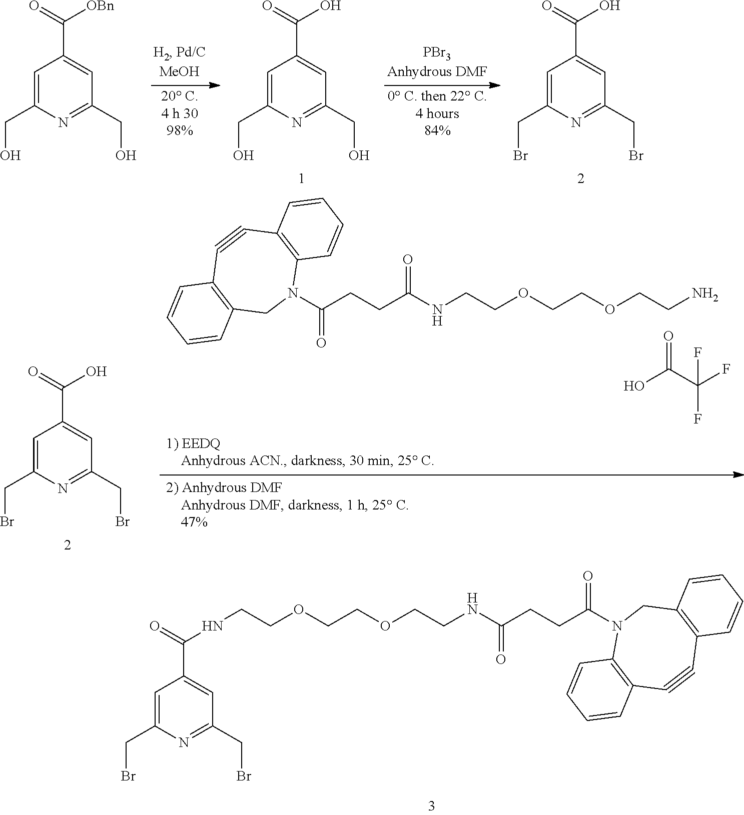

Step 1: preparation of 2,6-bis(hydroxymethyl)isonicotinic acid 1

Benzyl 2,6-bis(hydroxymethyl)isonicotinoate (1330 mg; 4.867 mmol; 1.0 eq) was dissolved in MeOH (50 mL) then the solution was degassed with argon for 15 minutes. Pd/C at 10% by mass (133 mg; 10% m/m) was added and the mixture was stirred at 20° C. under a hydrogen atmosphere for 4 hours 30 minutes. The reaction medium was filtered over Dicalite™ (MeOH rinsing). The filtrate was concentrated under reduced pressure in order to give 2,6-bis(hydroxymethyl)isonicotinic acid 1 (874 mg; 98%) in the form of an off-white solid.

¹H NMR (300 MHz, DMSO-d₆) δ 7.78 (s; 2H); 5.54 (br s; 2H); 4.59 (s; 4H).

¹³C NMR (75 MHz, DMSO-d₆) δ 166.7 (1C); 162.5 (2C); 139.4 (1C); 117.3 (2C); 64.0 (2C).

HRMS (ESI): m/zcalculated for C₈H₉NO₄ [M]=183.0532; observed [M]=183.0526.

### Step 2: Preparation of 2,6-Bis(Bromomethyl)Isonicotinic Acid 2

Under an inert atmosphere, 2,6-bis(hydroxymethyl)isonicotinic acid 1 (170 mg; 0.928 mmol; 1.0 eq) was dissolved in anhydrous DMF (3 mL). The solution was cooled to 0° C. and phosphorus tribromide (352 μL; 3.712 mmol; 4.0 eq) was added. The medium was allowed to return to 25° C. and stirred and chilled until complete conversion (for 4 hours). The crude medium was cooled to 0° C. and hydrolysed by addition of water. The precipitate formed was filtered and rinsed several times with cold water in order to give 2,6-bis(bromomethyl)isonicotinic acid 2 (240 mg; 84%) in the form of a beige solid.

¹H NMR (300 MHz, MeOD) δ 7.98 (s; 2H); 4.68 (s; 4H).

¹³C NMR (75 MHz, DMSO-d₆) δ 166.9 (1C); 159.7 (2C); 142.4 (1C); 123.5 (2C); 33.2 (2C).

HRMS (ESI): m/z calculated for CsHsBr₂NO₂ [M+H]⁺=307.8916; observed [M+H]⁺=307.8920.

Step 3: preparation of DBCO 2,6-bis(bromomethyl)isonicotamido-PEG₂-ethyl-carbamate 3

According to the general protocol for coupling with EEDQ, 2,6-bis(bromomethyl)isonicotinic acid (6.3 mg; 20.4 μmol; 2.2 eq) was dissolved in anhydrous ACN (1020 μL), then EEDQ (38.4 mg; 155.3 μmol; 16.4 eq) was added, protected from light. The medium was stirred at 25° C., protected from light for 30 minutes. A solution of TFA salt of amino-PEG-DBCO (5.2 mg; 9.5 μmol; 1.0 eq) dissolved in anhydrous DMF (865 μL) in the presence of anhydrous DIPEA (16.64 μL; 95.5 μmol; 10.1 eq) was added, protected from light. The medium was stirred for 1 hour at 25° C. The reaction crude was purified by semi-preparative HPLC (t_{R}=25 minutes) and DBCO 2,6-bis(bromomethyl)isonicotamido-PEG₂-ethyl-carbamate 3 was obtained in the form of a pale yellow lacquer (3.2 mg; 47%) after concentration and lyophilisation. ¹H NMR (300 MHz, MeOD) δ 7.79 (s; 2H); 7.65-7.55 (m; 2H); 7.48-7.42 (m; 3H); 7.40-7.26 (m; 2H); 7.25-7.19 (m; 1H); 5,12 (d; J=14.0 Hz; 1H); 4.62 (s; 4H); 3.70 (d; J=14.0 Hz; 2H); 3.67-3.53 (m; 8H); 3.48-3.40 (m; 2H); 3.26-3.18 (m; 2H); 2.79-2.60 (m; 1H); 2.39-2.27 (m; 1H); 2.24-2.09 (m; 1H); 2.05-1.90 (m; 1H).

HRMS (ESI): m/z calculated for C₃₃H3₅Br₂N40₅ [M+H]⁺=725.0969; observed [M+H]⁺=725.0959.

Analytical HPLC: t_{R}=5.592 minutes; 93%.

### Example 2: DBCO 2,6-bis(bromomethyl)isonicotamido-PEG₄-ethyl-carbamate 4

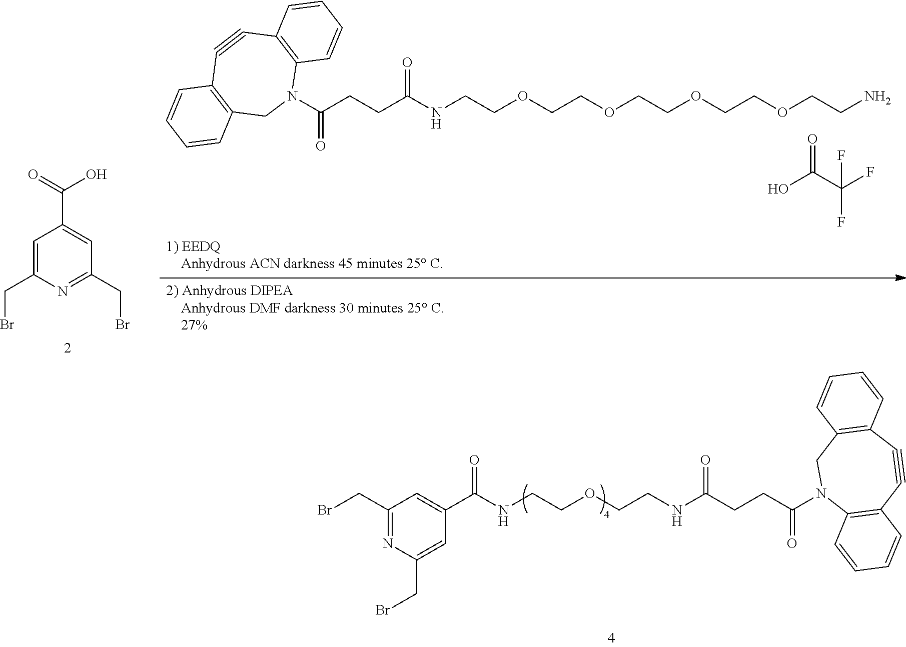

According to the general protocol for coupling with EEDQ, 2,6-bis(bromomethyl)isonicotinic acid 2 (4.1 mg; 13.3 μmol; 1.7 eq) was dissolved in anhydrous ACN (880 μL), then EEDQ (25.1 mg; 101.4 μmol; 13.0 eq) was added, protected from light. The medium was stirred at 25° C., protected from light for 45 minutes. A solution of the salt of TFA of DBCO amino-PEG₄-ethyl-carbamate (5.0 mg; 7.8 μmol; 1.0 eq) in anhydrous DMF (745 μL) in the presence of anhydrous DIPEA (10.8 μL; 62.4 μmol; 8,0 eq) was added, protected from light. The medium was stirred for 1 hour at 25° C. The reaction crude was purified by semi-preparative HPLC (t_{R}=25 minutes) and DBCO 2,6-bis(bromomethyl)isonicotamido-PEG₄-ethyl-carbamate 4 was obtained in the form of a pale yellow solid (1.7 mg; 27%) after concentration and lyophilisation. ¹H NMR (300 MHz, MeOD) δ 7.82 (s; 2H); 7.65-7.57 (m; 2H); 7.48-7.43 (m; 3H); 7.38-7.28 (m; 2H); 7.26-7.22 (m; 1H); 5.35 (m; 1H); 5.12 (d; J=13.9 Hz; 1H); 4.65-4.63 (m; 6H); 3.70 (d; J=13.9 Hz; 1H); 3.68-3.51 (m; 14H); 3.40-3.37 (m; 2H); 3.21 (t; J=5.8 Hz; 2H); 2.74-2.64 (m; 1H); 2.41-2.31 (m; 1H); 2.22-2.12 (m; 1H); 2.08-1.96 (m; 1H).

HRMS (ESI): m/z calculated for C₃₇H₄₃Br₂N₄O₇ [M+H]⁺=813.1493; observed [M+H]⁺=813.1480. analytical HPLC: t_{R}=5.575 minutes; 88%.

### Example 3: 2,6-bis(bromomethyl)isonicotamido-PEG₄-BCN 6

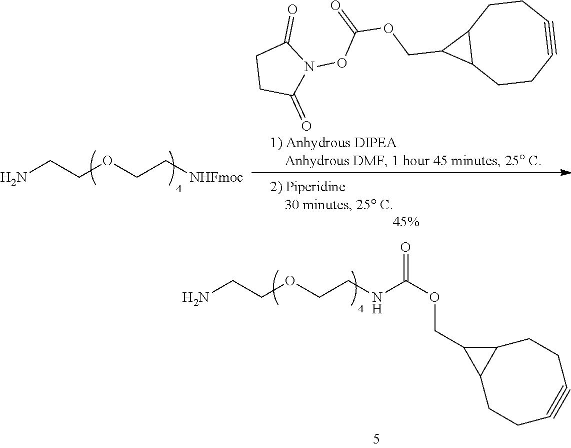

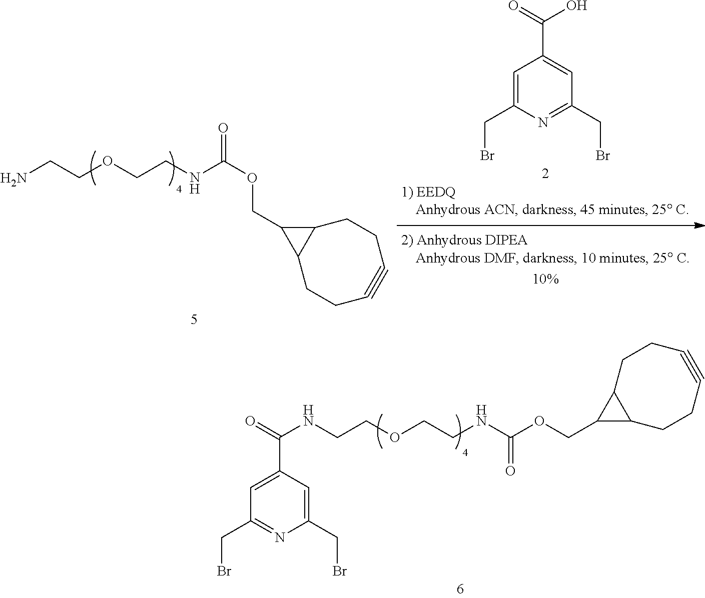

Step 1: preparation of BCN amino-PEG₄-ethyl-carbamate 5

BCN N-hydroxysuccinimide ester (20 mg; 68,7 μmol; 1.0 eq) was dissolved in anhydrous DMF (1 mL), then amino-PEG₄-ethylenaminofluorenylmethoxycarbonyl chloride (34 mg; 68.7 μmol; 1.0 eq) and anhydrous DIPEA (35.9 μL; 206.1 μmol; 3.0 eq) were added. The reaction medium was stirred at 28° C. until complete conversion (for 1 hour 45 minutes). Piperidine was added, (100 μL; 10% v/v) and the medium was stirred at 25° C. for 30 minutes. The reaction crude was purified by semi-preparative HPLC without acid in the mobile phase (t_{R}=33 minutes) and BCN amino-PEG₄-ethyl-carbamate 5 was obtained after concentration and lyophilisation, in the form of a colourless oil (12.5 mg; 45%). ¹H NMR (300 MHz, MeOD) δ 4.17 (d; J=8.2 Hz; 2H); 3.70-3.62 (m; 12H); 3.58-3.53 (m; 4H); 3.30 (t; J=5.0 Hz; 2H); 2.84 (t; J=5 Hz; 2H); 2.33-2.14 (m; 6H); 1.70-1.58 (m; 2H); 1.41 (q; J=8.5 Hz; 1H); 1.03-0.93 (m; 2H). ¹³C NMR (75 MHz, MeOD) δ 159.3 (1C); 99.5 (1C); 73.0-71.1 (8C); 63.7 (1C); 42.0 (1C); 41.7 (1C); 30.2 (2C); 21.9 (2C); 21.4 (2C); 19.0 (1C).

HRMS (ESI): m/zcalculated for C₂₁H₃₆N₂O₆[M]=412.2573; observed [M]=412.2583.

Step 2: preparation of 2,6-bis(bromomethyl)isonicotamido-PEG₄-BCN 6

Under an inert atmosphere, 2,6-bis(bromomethyl)isonicotinic acid 2 (15.9 mg; 51.5 μmol; 1.7 eq) was dissolved in anhydrous ACN (1.7 mL), then EEDQ (97.4 mg; 393.9 μmol; 13.0 eq) was added, protected from light. The medium was stirred at 25° C., protected from light for 45 minutes. A solution of BCN amino-PEG₄-ethyl carbamate 5 (12.5 mg; 30.3 μmol; 1.0 eq) in anhydrous DMF (1.4 mL) in the presence of anhydrous DIPEA (42 μL; 242.4 μmol; 8.0 eq) was added, protected from light. The medium was stirred for 10 minutes at 25° C. The reaction crude was purified by semi-preparative HPLC without method in the mobile phase (t_{R}=33.5 minutes) and 2,6-bis(bromomethyl)isonicotamido-PEG₄-BCN 6 was obtained after concentration and lyophilisation, in the form of a pale yellow lacquer (2.1 mg; 10%). ¹H NMR (300 MHz, MeOD) δ 7.84 (s; 2H); 4.65 (s; 4H); 4.13 (d; J=8.1 Hz; 2H); 3.70-3.55 (m; 20H); 3.50 (t; J=5.5 Hz; 2H); 3.28-3.23 (m; 2H); 2.29-2.11 (m; 6H); 1.66-1.55 (m; 2H); 1.41-1.35 (m; 1H); 0.96-0.87 (m; 2H).

HRMS (ESI): m/z calculated for C₂₉H₄₂Br₂N₃O₇ [M+H]⁺=702.1384; observed [M+H]⁺=702.1372.

Analytical HPLC: t_{R}=5.067 minutes; 91%.

### Example 4: 2,6-bis(bromomethyl)isonicotamido-PEG₂-TCO 7

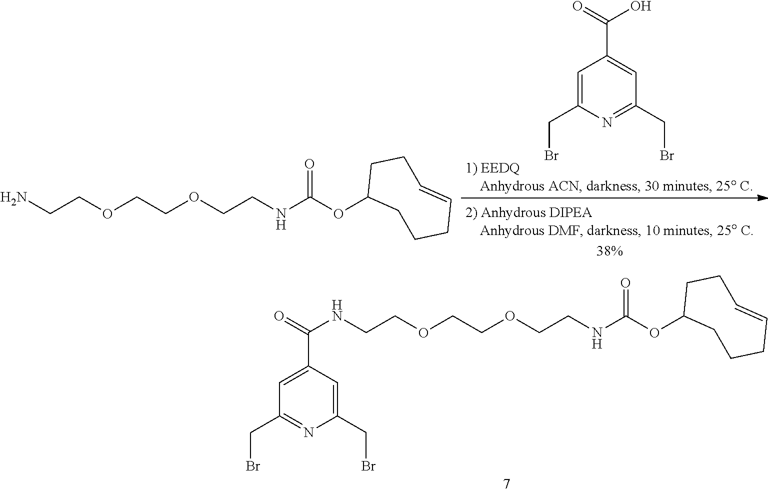 [SMILES: O=C(O)c1cc(CBr)nc(CBr)c1 ; NCCOCCOCCNC(=O)OC1CC/C=C/CCC1 ; O=C(NCCOCCOCCNC(=O)c1cc(CBr)nc(CBr)c1)OC1CC/C=C/CCC1]

According to the general protocol for coupling with EEDQ, 2,6-bis(bromomethyl)isonicotinic acid 2 (21.6 mg; 69.9 μmol; 2.1 eq) was dissolved in anhydrous ACN (1.0 mL), then EEDQ (41.2 mg; 166.5 μmol; 5.0 eq) was added, protected from light. The medium was stirred at 25° C., protected from light for 30 minutes. A solution of TCO amino-PEG₂-ethyl carbamate (10.0 mg; 33.3 μmol; 1.0 eq) in anhydrous DMF (250 μL) in the presence of anhydrous DIPEA (23.2 μL; 133.2 μmol; 4,0 eq) was added, protected from light. The medium was stirred for 10 minutes at 25° C. The reaction crude was purified by semi-preparative HPLC (t_{R}=27.8 min) and 2,6-bis(bromomethyl)isonicotamido-PEG₂-TCO 7 was obtained after concentration and lyophilisation, in the form of a pale yellow lacquer (7.5 mg; 38%). ¹H NMR (300 MHz, MeOD) δ 8,85 (m; 1H); 7.84 (s, 2H); 5.68-5.40 (m; 2H); 4.65 (s; 4H); 4.32-4.25 (m; 1H); 3.70-3.57 (m; 8H); 3.54-3.49 (m; 2H); 3.27-3.22 (m; 2H); 2.35-2.26 (m; 2H); 2.00-1.85 (m; 4H); 1.77-1.50 (m; 4H).

HRMS (ESI): m/z calculated for C₂₃H₃₄Br₂N₃O₅ [M+H]⁺=590.0860; observed [M+H]⁺=590.0860.

### Example 5: DBCO methyltetrazin-PEG₄-amido-PEG₂-ethylcarbamate 8

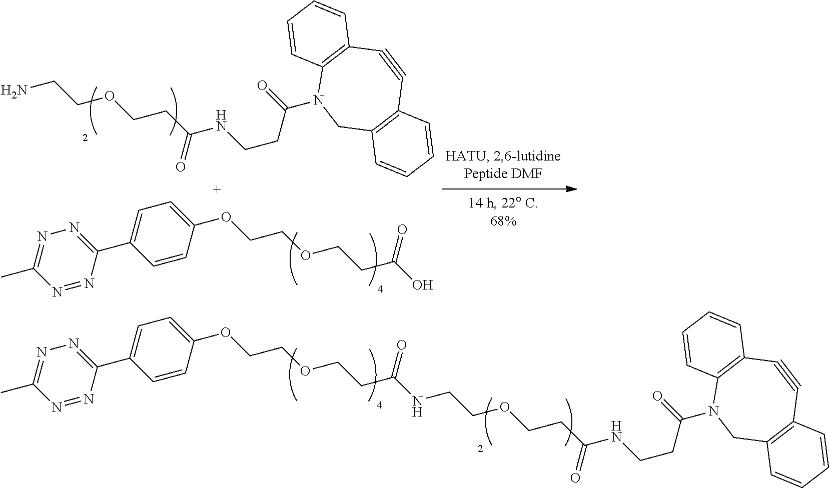

Methyltetrazine-PEG₄-ethylic acid (11.0 mg; 25.3 μmol; 1.4 eq) was dissolved in DMF (230 μL), then HATU (17.5 mg; 46.0 μmol; 2.0 eq) and 2,6-lutidine (10.66 μL; 92.0 μmol; 4.0 eq) were added. The medium was stirred at 22° C. for 10 minutes, then a solution of DBCO amino-PEG₂-ethyl carbamate in the form of TFA salt (10.0 mg; 18.2 μmol; 1.0 eq) in DMF (230 μL) was added. The medium was stirred for 14 hours at 22° C. The reaction crude was purified by semi-preparative HPLC (t_{R}=25.0 min) and DBCO methyltetrazine-PEG₄-amido-PEG₂-ethylcarbamate 8 was obtained after concentration and lyophilisation, in the form of a pink lacquer (13.7 mg; 88%). ¹H NMR (300 MHz, CDCl₃) δ 8,52 (dt; J=9.5; 2.0 Hz; 2H); 7.65 (dd; J=6.7; 1.6 Hz; 1H); 7.53-7.47 (m; 1H); 7.40-7.28 (m; 5H); 7.25-7.22 (m; 1H); 7.08 (dt; J=9.5; 2.0 Hz; 2H); 6.83-6.79 (m; 1H); 6.32-6.28 (m; 1H); 5.14 (d; J=14.3 Hz; 1H); 4.22 (t; J=5.4 Hz; 2H); 3.89 (t, J=4.8 Hz; 2H); 3.75-3.51 (m; 24H); 3.47-3.48 (m; 4H); 3.35-3.30 (m; 2H); 3.05 (s; 3H) under water; 2.86-2.75 (m; 1H); 2.53-2.39 (m; 3H); 2.18 (dt; J=15.1; 6.0 Hz; 1H); 1.94 (dt; J=19.9; 6.0 Hz; 1H).

HRMS (ESI): m/z calculated for C₄₅H₅₆N₇O₁₀ [M+H]⁺=854.4083; observed [M+H]⁺=854.4091.

Analytical HPLC: t_{R}=5.458 min; 94%.

### Example 6: Bioconjugation of Trastuzumab Fab (TTZ Fab)

Trastuzumab Fab was obtained by digestion of trastuzumab IgG with IgdE or papain, with mass of 47,499 Da and 47,637 Da respectively.

Trastuzumab Fab was used at a concentration between 0.10 mg/mL and 10 mg/mL in the bioconjugation buffer, for example 0.67 mg/mL.

Under an inert atmosphere, the reducing agent (4 to 100 eq, for example 8 eq) was added to trastuzumab Fab in the bioconjugation buffer (5 to 5000 μg, for example 33.5 μg; 1 eq), and the reaction medium were incubated at between 15 and 40° C., for example 37° C., for 0.25 to 5 hours, for example 2 hours. The solution of compound 3, 4, 6 or 7 (4 to 100 eq, for example 8 eq) was then added under an inert atmosphere and the reaction medium was stirred at between 15 and 40° C., for example 37° C., for 0.5 to 15 hours, for example 2.5 hours and the final product is purified by steric exclusion and concentrated if necessary.

Compound 9: TTZ Fab—compound 3 conjugate

### Denaturing MS Analysis

TABLE 2

| | Expected mass (Da) | Obtained mass (Da) | Relative intensity |
|---|---|---|---|
| MAR 0 | 47499 | — | — |
| MAR 1 | 48065 | 48066 | 100% |

Average MAR = 1.00

### Compound 10: TTZ Fab—Compound 4 Conjugate

### Denaturing MS Analysis

TABLE 3

| | Expected mass (Da) | Obtained mass (Da) | Relative intensity |
|---|---|---|---|
| MAR 0 | 47499 | — | — |
| MAR 1 | 48153 | 48154 | 100% |

Average MAR = 1.00

### Compound 11: TTZ Fab—Compound 6 Conjugate

### Denaturing MS Analysis

TABLE 4

| | Expected mass (Da) | Obtained mass (Da) | Relative intensity |
|---|---|---|---|
| MAR 0 | 47499 | — | — |
| MAR 1 | 48042 | 48043 | 100% |

Average MAR = 1.00

### Compound 12: TTZ Fab—Compound 7 Conjugate

### Denaturing MS Analysis

TABLE 5

| | Expected mass (Da) | Obtained mass (Da) | Relative intensity |
|---|---|---|---|
| MAR 0 | 47637 | 47638 | 6% |
| MAR 1 | 48068 | 48068 | 94% |

Average MAR = 0.94

### Example 7: Bioconjugation of Fab 1E12

1E12 Fab was obtained by digestion of the IgG 1E12 with IgdE. 1E12 Fab was used at a concentration between 0.10 mg/mL and 10 mg/mL in the bioconjugation buffer, for example 0.34 mg/mL.

Under an inert atmosphere, the reducing agent (4 to 100 eq, for example 8 eq) was added to 1E12 Fab in the bioconjugation buffer (5 to 5000 μg, for example 17.0 μg; 1 eq), and the reaction medium was incubated at between 15 and 40° C., for example 37° C., for 0.25 to 5 hours, for example 2 hours. The solution of compound 3, 4 or 6 (4 to 100 eq, for example 8 eq) was then added under an inert atmosphere and the reaction medium was stirred at between 15 and 40° C., for example 37° C., for 0.5 to 15 hours, for example 2.5 hours and the final product is purified by steric exclusion and concentrated if necessary.

### Compound 13: 1E12 Fab—Compound 3 Conjugate

### Denaturing MS Analysis

TABLE 6

| | Expected mass (Da) | Obtained mass (Da) | Relative intensity |
|---|---|---|---|
| MAR 0 | 48144 | — | — |
| MAR 1 | 48710 | 48710 | 100% |

Average MAR = 1.00

### Compound 14: 1E12 Fab—Compound 4 Conjugate

Denaturing MS analysis

TABLE 7

| | Expected mass (Da) | Obtained mass (Da) | Relative intensity |
|---|---|---|---|
| MAR 0 | 48144 | — | — |
| MAR 1 | 48798 | 48799 | 100% |

Average MAR = 1.00

### Compound 15: 1E12 Fab—Compound 6 Conjugate

### Denaturing MS Analysis

TABLE 8

| | Expected mass (Da) | Obtained mass (Da) | Relative intensity |
|---|---|---|---|
| MAR 0 | 48144 | — | — |
| MAR 1 | 48687 | 48687 | 100% |

Average MAR = 1.00

### Example 8: Synthesis of Reaction Intermediates

### Preparation of 4-Methoxyphenyl 2-Azidoacetate 16

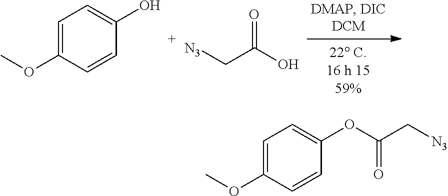

4-methoxyphenol (98.4 mg; 0.79 mmol; 1.0 eq) was dissolved in DCM (2.5 mL), and 4-dimethylaminopyridine (9.8 mg; 0.08 mmol; 0.1 eq) was added to the reaction medium. In parallel, 2-azidoacetic acid (71.2 μL; 0.95 mmol; 1.2 eq) was dissolved in DCM (2.5 mL), and N,N′-diisopropylcarbodiimide (147.3 μL; 0.95 mmol; 1.2 eq) was added to this second solution. The two solutions were stirred at 22° C. for 15 minutes. The second solution was added, gradually to the first then the reaction medium was stirred at 22° C. for 16 hours. The solution was concentrated under reduced pressure. The residue was purified by flash chromatography (12 g of silica, DCM 100%) in order to obtain 4-methoxyphenyl 2-azidoacetate 16 (97.4 mg; 59%) in the form of a yellow oil.

¹H NMR (300 MHz, CDCl₃) δ 7.08-7.03 (m; 2H); 6.93-6.88 (m; 2H); 4.12 (s; 2H); 3.81 (s; 3H).

¹³C NMR (75 MHz, CDCl₃) δ 167.34 (1C); 157.80 (1C); 143.74 (1C); 122.11 (2C); 114.74 (2C); 55.77 (1C); 50.59 (1C).

HRMS (ESI): m/z calculated for C₉H₉N₃O₃Na [M+Na]⁺=230.0541; observed [M+Na]⁺=230.0536.

Preparation of 4-methoxyphenyl 1-azido-13-oxo-3,6,9-trioxa-12-azahexadecan-16-oate 18

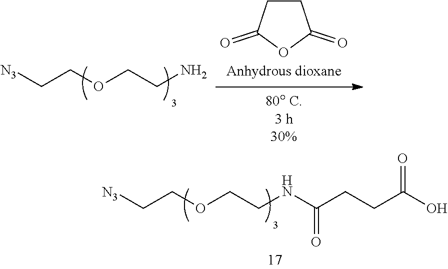

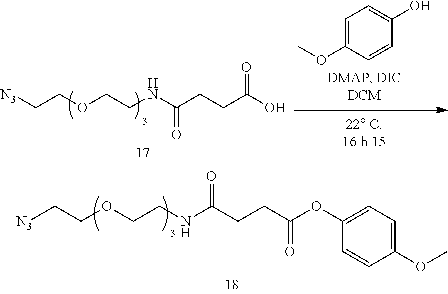

a) Preparation of 1-azido-13-oxo-3,6,9-trioxa-12-azahexadecan-16-oic acid 17

11-azido-3,6,9-trioxaundecan-1-amine (74.1 mg; 0.34 mmol; 1.0 eq) was dissolved in anhydrous dioxane (1.5 mL), and succinic anhydride (34.0 mg; 0.34 mmol; 1.0 eq) was added to the reaction medium. The reaction medium was stirred at 80° C. for 3 hours. After returning to AT and dilution in ethyl acetate, the organic phase was washed with water (3×10 mL) and with a saturated solution of NaCl (1×10 mL) then dried over MgSO₄ and concentrated under reduced pressure. The solution was concentrated under reduced pressure. The residue was purified by flash chromatography (4 g of silica, DCM/MeOH 90:10) in order to obtain 1-azido-13-oxo-3,6,9-trioxa-12-azahexadecan-16-oic acid 17 (30.0 mg; 28%) in the form of a yellow oil.

¹H NMR (300 MHz, CDCl₃) δ 6.64 (m; 1H); 3.68-3.60 (m; 10H); 3.56-3.53 (m; 2H); 3.46-3.37 (m; 4H); 2.69-2.63 (m; 2H); 2.53-2.49 (m; 2H).

¹³C NMR (75 MHz, CDCl₃) δ 175.64 (1C); 172.58 (1C); 70.75 (1C); 70.69 (1C); 70.56 (1C); 70.27 (1C); 70.07 (1C); 69.71 (1C); 50.76 (1C); 39.57 (1C); 31.00 (1C); 30.12 (1C).

b) Preparation of 4-methoxyphenyl 1-azido-13-oxo-3,6,9-trioxa-12-azahexadecan-16-oate 18

4-methoxyphenol (9.7 mg; 0.08 mmol; 1.0 eq) was dissolved in DCM (1.0 mL), and 4-dimethylaminopyridine (1.1 mg; 0.009 mmol; 0.1 eq) was added to the reaction medium. In parallel, 1-azido-13-oxo-3,6,9-trioxa-12-azahexadecan-16-oic acid 17 (30.0 mg; 0.09 mmol; 1.2 eq) was dissolved in DCM (1.0 mL), and N,N′-diisopropylcarbodiimide (14.6 μL; 0.09 mmol; 1.2 eq) was added to this second solution. The two solutions were stirred at 22° C. for 15 minutes. The second solution was carefully added to the first, then the reaction medium was stirred at 22° C. for 16 hours. After recovery in water, the aqueous phase was extracted by DCM (3×10 mL) then the organic phase was washed with water (1×10 mL) and with a saturated solution of NaCl (1×10 mL) then dried over MgSO₄ and concentrated under reduced pressure. The residue was purified by flash chromatography (12 g of silica, DCM/MeOH 90:10) in order to obtain 4-methoxyphenyl 1-azido-13-oxo-3,6,9-trioxa-12-azahexadecan-16-oate 18 in the form of a yellow oil. ¹H NMR (300 MHz, CDCl₃) δ 7.04-6.99 (m; 2H); 6.90-6.86 (m; 2H); 6.33 (bs; 1H); 3.80 (s; 3H); 3.71-3.61 (m; 10H); 3.58-3.55 (m; 2H); 3.50-3.47 (m; 2H); 3.41-3.38 (m; 2H); 2.91 (t; J=6.9 Hz; 2H); 2.60 (t; J=6.9 Hz; 2H). ¹³C NMR (75 MHz, CDCl₃) δ 172.03 (1C); 171.23 (1C); 157.34 (1C); 144.32 (1C); 122.40 (2C); 114.52 (2C); 70.80 (1C); 70.72 (1C); 70.66 (1C); 70.38 (1C); 70.14 (1C); 69.91 (1C); 55.69 (1C); 50.79 (1C); 39.48 (1C); 30.95 (1C); 29.72 (1C).

HRMS (ESI): m/z calculated for C₁₉H₂₉N₄O₇ [M+H]⁺=425.2035; observed [M+H]⁺=425.2031.

### Example 9: Functionalisation of Proteases

Proteases, IdeS—TAG His (GSSHHHHHH) or IdeS, were used at a concentration between 0.5 and 10 mg/mL in HEPES buffer, for example 1 mg/mL.

To protease IdeS or IdeS—TAG His (GSSHHHHHH) in the HEPES buffer (25 to 5000 for example 500 μg, 1 eq) was added the solution of compound 16 or 18 (50 to 500 eq, for example 222 eq). The reaction medium was stirred or not stirred, at between 0 and 37° C., for example 4° C., for 1 to 48 hours, for example 16 hours and the product was purified by steric exclusion.

Compound 19: Compound 16—protease IdeS—TAG His (GSSHHHHHH)

### Denaturing MS Analysis

TABLE 9

| | Expected mass (Da) | Obtained mass (Da) | Relative intensity |
|---|---|---|---|
| MPR 0 | 37432 | 37433 | 26% |
| MPR 1 | 37516 | 37515 | 74% |

Average MPR = 0.74

Compound 20: Compound 16—protease IdeS

### Denaturing MS Analysis

TABLE 10

| | Expected mass (Da) | Obtained mass (Da) | Relative intensity |
|---|---|---|---|
| MPR 0 | 35682 | 35682 | 55% |
| MPR 1 | 35766 | 35764 | 45% |

Average MPR = 0.45

Compound 21: Compound 18—protease IdeS—TAG His (GSSHHHHHH)
Denaturing MS analysis

TABLE 11

| | Expected mass (Da) | Obtained mass (Da) | Relative intensity |
|---|---|---|---|
| MPR 0 | 37432 | 374323 | 88% |
| MPR 1 | 37733 | 37732 | 12% |

Average MPR = 0.12

Compound 22: Compound 8—compound 16—protease IdeS—TAG His (GSSHHHHHH)

Compound 19 (100.0 μg; 1 eq) was reacted in the presence of compound 8 (1 to 10 eq, for example 8 eq). The reaction mixture was stirred at between 4 and 40° C., for example 25° C., for 1 to 96 hours, for example 16 hours, in order to obtain compound 22.

Denaturing MS analysis

TABLE 12

| | Expected mass (Da) | Obtained mass (Da) | Relative intensity |
|---|---|---|---|
| MPR 0 | 37432 | 37432 | 29% |
| MPR 1 | 38370 | 38370 | 71% |

Average MPR = 0.71

### Example 10: Click Reaction

Use of two click partners: 19 or 22 with 9, 10, 11, 13, 14 or 15.

Each of the click partners was concentrated:
Either using a Vivaspin™ 10 kDa centrifugal concentrator with centrifuge (5000 G) for the IdeS compounds in order to attain a concentration between 0.5 and 10 mg/mL, for example 1 mg/mL.
or using an Amicon® Ultra 10 kDa centrifugal concentrator with centrifuge (10,000 G) for the Fab compounds in order to attain a concentration between 0.2 and 10 mg/mL, for example 0.4 mg/mL.

TABLE 13

| Conjugated fragment | +modified protease | → Clicked conjugate |
|---|---|---|
| 9 | 19 | 23 |
| 10 | | 24 |
| 11 | | 25 |
| 13 | | 26 |
| 14 | | 27 |
| 15 | | 28 |
| 11 | 22 | 29 |

The protease partner (1 to 10 eq, for example 2 eq) was reacted in the presence of the fragment partner (1 to 10 eq, for example 1 eq). The reaction mixture was stirred at between 4 and 40° C., for example 4° C., for 1 to 96 hours, for example 16 hours.

### Compound 23

Compound 9 (10 μL-8,32 μM-0.4 mg/mL-1 eq)—Compound 19 (6.24 μL-26.7 μM-1 mg/mL-2 eq).

Denaturing MS analysis

TABLE 14

| | Expected mass (Da) | Obtained mass (Da) |
|---|---|---|
| Click | 85581 | 85586 |

1.00 protease per fragment.

Compound 24 Compound 10 (10 μL-0.74 mg/mL-1 eq)—Compound 19 (2.6 mg/mL-2 eq).
Denaturing MS analysis

TABLE 15

| | Expected mass (Da) | Obtained mass (Da) |
|---|---|---|
| Click | 85669 | 85672 |

1.00 protease per fragment.

### Compound 25

Compound 11 (15 μL-0.40 mg/mL-1 eq)—Compound 19 (3.1 mg/mL-4 eq).

Denaturing MS analysis

TABLE 16

| | Expected mass (Da) | Obtained mass (Da) |
|---|---|---|
| Click | 85558 | 85561 |

1.00 protease per fragment.

### Compound 26

Compound 13 (15 μL-0.30 mg/mL-1 eq)—Compound 19 (3.1 mg/mL-4 eq).

### Denaturing MS Analysis

TABLE 17

| | Expected mass (Da) | Obtained mass (Da) |
|---|---|---|
| Click | 86225 | 86227 |

1.00 protease per fragment.

### Compound 27

Compound 14 (15 μL-0.30 mg/mL-1 eq)—Compound 19 (3.1 mg/mL-4 eq).

Denaturing MS analysis

TABLE 18

| | Expected mass (Da) | Obtained mass (Da) |
|---|---|---|
| Click | 86313 | 86315 |

1.00 protease per fragment.

### Compound 28

Compound 15 (15 μL-0.30 mg/mL-1 eq)—Compound 19 (3.1 mg/mL-4 eq).

Denaturing MS analysis

TABLE 19

| | Expected mass (Da) | Obtained mass (Da) |
|---|---|---|
| Click | 86202 | 86203 |

1.00 protease per fragment.

### Compound 29

Compound 11 (15 μL-0.60 mg/mL-1 eq)—Compound 22 (0.56 mg/mL-4 eq).

Denaturing MS analysis

TABLE 20

| | Expected mass (Da) | Obtained mass (Da) |
|---|---|---|
| Click | 86412 | 86419 |

1.00 protease per fragment.

### Example 11: Proteolytic Activity of Compound 24

### a) Purification of Compound 24

Compound 24 was purified by steric exclusion chromatography on a UHPLC Vanquish™ Flex system, having a quaternary pump, using an Agilent BioSEC-5 column; 5 μM; 150 Å; 7.8×300 mm positioned in a thermostatic oven at 25° C. by stationary air. The elution was performed with PBS buffer (KH₂PO₄ 1 mM, NaCl 205 mM, Na₂HPO₄.7H₂O₃ mM, pH 7.0), a flow rate of 0.5 mL/min was used. The various fractions are detected at 280 nm (HL Vanquish™ diode array detector). The fractions corresponding to compound 24 (t_{R}=13.3 minutes) were combined and concentrated by Vivaspin™ in order to obtain a concentration of approximately 0.2 mg/mL.

The purity of compound 24 was determined by SDS-PAGE gel and densiometric analysis.

TABLE 21

| | Optical density (%) |
|---|---|
| Compound 24 | 85 |
| Compound 10 | 15 |
| Compound 19 | 0 |

### b) Evaluation of the Proteolytic Activity

A sample of IdeS having been subjected to the same conditions as compound 24, but without chemical modification, was used as control (Control). 2.5 μL of Control or of compound 24 at 0.27 μM in PBS Gibco® were added to 56.3 μL of trastuzumab at 3.33 mg/mL. The reaction media were incubated at 37° C. for 15 minutes then analysed by SDS-PAGE gel and densitometry.

TABLE 22

| | Control | Compound 24 |
|---|---|---|
| trastuzumab | 11% | 14% |
| simple cleaved trastuzumab | 67% | 66% |
| F(ab′)₂ | 4% | 8% |
| Fc residues | 18% | 12% |

Compound 24 has a proteolytic activity similar to the Control.

### c) Evaluation of Antigen Recognition

The various samples (TTZ Fab and compound 24) were diluted to a concentration of 1 μM in PBS Gibco 1× buffer. The antigen HER2 (Interchim) was immobilised overnight at 4° C. in a 96-well plate (ThermoScientific) at a concentration of 1 μg/mL (100 μL per well). The wells were then saturated with 300 μL per well of 3% BSA solution in PBS for 1 hour at 37° C. The samples were deposited in the wells with decreasing concentrations from 100 to 0.001 nM (100 μL per well) then incubated for 1 hour at 37° C. After 4 successive washings with PBS at 0.05% Tween, the protein L-HRP (ThermoFisher, 0.25 μg/μL diluted to 1/800) was deposited in the wells (100 μL) and allowed to incubate for 1 hour at 37° C. 3,3′,5,5′-tetramethylbenzidine (100 μL per well) was added, and allowed to react for several minutes. Finally, 1 M H₂SO₄ (50 μL per well) was added, in order to stop the reaction and the absorbance of the 96-well plates was read at 450 nm using the Multiskan system (ThermoScientific) and ELISA ascent software for iEMS Reader MF.

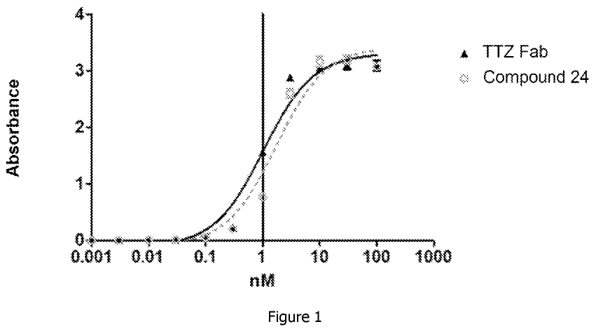

The recognition kinetics for antigen HER2 by TTZ Fab and compound 24 are presented in FIG. 1. These data have made it possible to determine the affinity constant of TTZ Fab and compound 24.

TABLE 23

| | TTZ Fab | Compound 24 |
|---|---|---|
| Affinity constant (nM) | 1.04 | 1.71 |

Compound 24 according to the invention recognises the antigen HER2 in a similar manner to compound TTZ Fab.

### REFERENCES

[1] Barran, P. et al., EuPA Open Proteomics, 11, 23-27, 2016
[2] Vayne, C. et al., Thrombosis and Haemostasis, 121(03), 322-331, 2021
[3] Kinder et al., The Journal of Biological Chemistry, vol. 288, no. 43, pp. 30843-30854, 2013

1. A conjugate comprising:
a) an antibody or an antibody fragment, and
b) a specific protease for the immunoglobulin G (IgG) hinge region.

2. The conjugate according to claim 1, of formula (A): 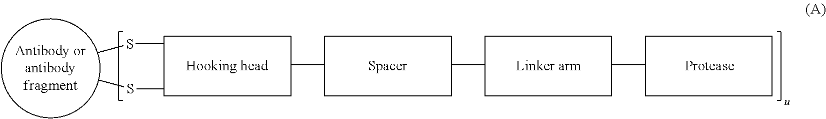
wherein u=1, 2, 3, 4, 5 or 6.

3. The conjugate according to claim 1, of formula (A): 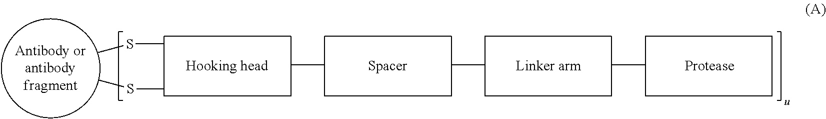
wherein u=1, 2, 3, 4, 5 or 6,
and wherein the hooking head is a compound of formula (I): 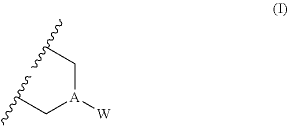
wherein:
A is the residue of a phenyl or a pyridyl;
W is —ORₐ, —COR₂, —CONR₃R₄ or —NR₃COR₄;
Rₐ is —(CH₂CH₂O)_{q}—(CH₂)ᵣ-R₅-, —(CHR_{c})ᵣ-R₅-, —COR_{b}, —(CHR_{c})ᵣ—NHCO—(CH₂CH₂O)_{q}—(CH₂)ᵣ-R₅- or —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣ-R₅— or —(CH₂CH₂O)_{q}—(CH₂)ᵣ—CONH—(CHR_{c})ᵣ-R₅-;
R_{b} is —(CH₂CH₂O)_{q}—(CH₂)ᵣ-R₅-, —O(CH₂CH₂O)_{q}—(CH₂)ᵣ-R₅-, —(CHR_{c})ᵣ-R₅-, —O(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣR₅- or —(CH₂CH₂O)_{q}—(CH₂)ᵣ—CONH—(CHR_{c})ᵣ-R₅-;
R₂ is —(CH₂CH₂O)_{q}—(CH₂)ᵣ-R₅-, —(CHR_{c})ᵣ-R₅-, —O(CH₂CH₂O)_{q}—(CH₂)ᵣ-R₅-, —O(CHR_{c})ᵣ- R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)_{q}—(CH₂)ᵣ—NHCO—(CR_{c}H)ᵣ-R₅- or —O(CH₂CH₂O)_{q}—(CH₂)ᵣ—CONH—(CHR_{c})ᵣ-R₅-;
R₃ is —H, —(C₁-C₆)alkyl or —(CH₂)ᵥ—SO₃H;
R₄ is —(CH₂CH₂O)_{q}—(CH₂)ᵣ-R₅-, —(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣ-R₅— or —(CH₂CH₂O)_{q}—(CH₂)ᵣ—CONH—(CHR_{c})ᵣ-R₅-;
R₅ is chosen from: 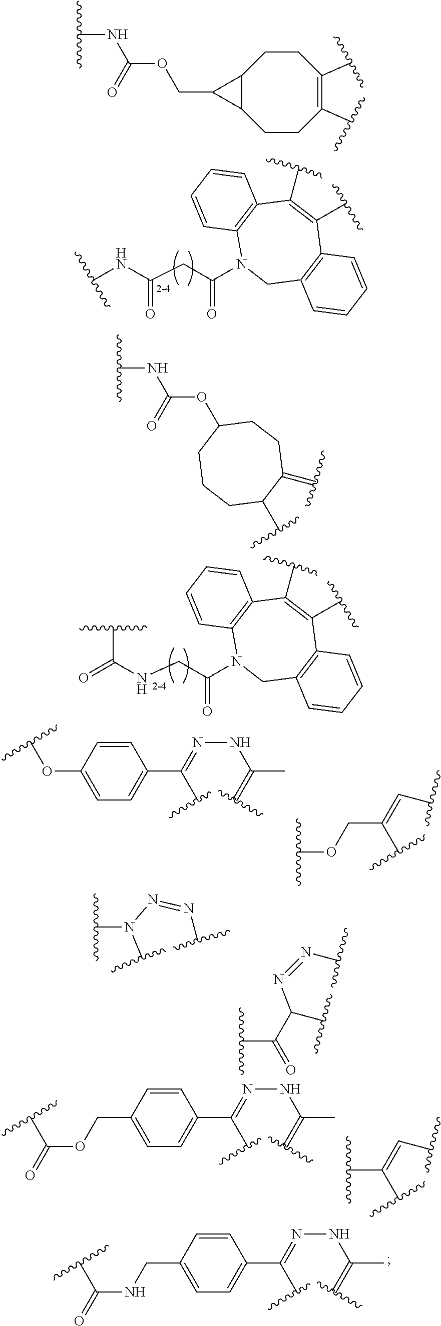
each R_{c} is chosen from —H, —CH₂—SO₃H or —SO₃H;
q is an integer ranging from 1 to 24;
each r is an integer ranging from 1 to 8;
each v is an integer ranging from 1 to 6.

4. The conjugate according to claim 1, of formula (A): 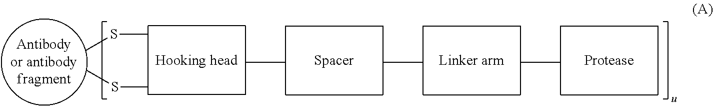
wherein u=1, 2, 3, 4, 5 or 6,
and wherein the spacer is a direct bond or a group of formula -(R₅-R₆-R₅′)ₛ wherein:
s is an integer ranging from 1 to 10
R₅ and R₅′ are different from one another and are each chosen from: 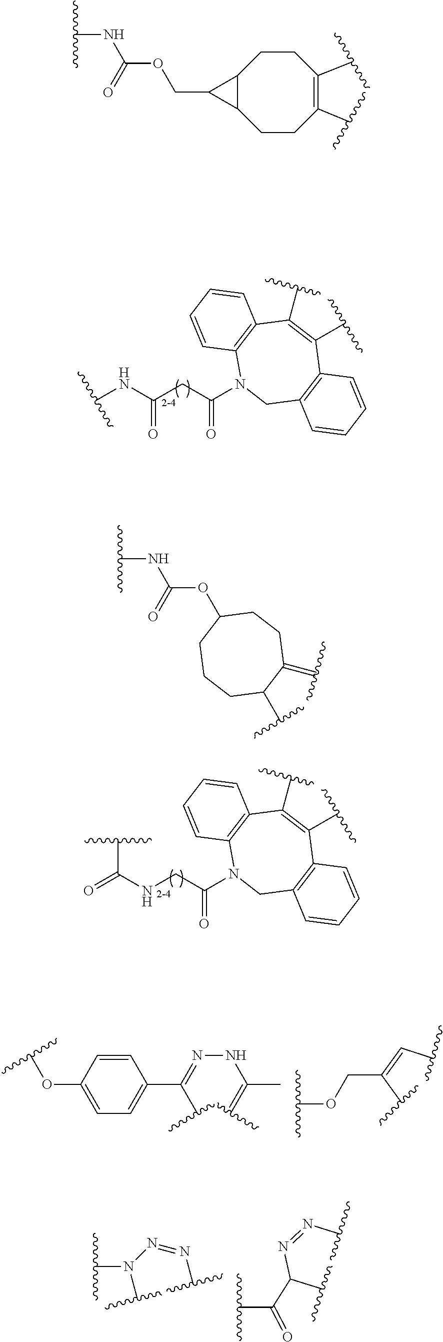 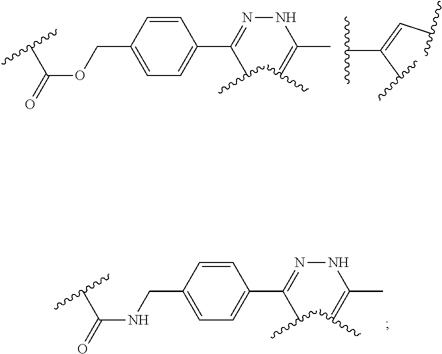
R₆ is —(CH₂CH₂O)_{q}—(CH₂)ᵣ—, —(CH₂CH₂O)_{q}—(CH₂)ᵣ—CO-R₇-NH—(CH₂CH₂O)_{q}—(CH₂)ᵣ—, —(CH₂CH₂O)_{q}—(CH₂)ᵣ—NH-R₈—CO—(CH₂CH₂O)_{q}—(CH₂)ᵣ—, —(CH₂)ᵣ—NHCO—(CHR_{c})ᵣ, —(CH₂CH₂O)_{q}—(CH₂)ᵣ—CONH—(CH₂CH₂O)_{q}—(CH₂)ᵣ—NHCO—(CH₂CH₂O)_{q}—(CH₂)ᵣ—, —(CH₂CH₂O)_{q}—(CH₂)ᵣ—CONH—(CHR_{c})ᵣ—NHCO—(CH₂CH₂O)_{q}—(CH₂)ᵣ—, —(CH₂CH₂O)_{q}—(CH₂)ᵣ NHCO—(CH₂CH₂O)_{q}—(CH₂)ᵣ—CONH—(CH₂CH₂O)_{q}—(CH₂)ᵣ or —(CH₂CH₂O)_{q}—(CH₂)ᵣ—NHCO—(CHR_{c})ᵣ—CONH—(CH₂CH₂O)_{q}—(CH₂)ᵣ—;
R₇ is a direct bond, —NH—(CH₂CH₂O)_{q}—(CH₂)ᵣ—CO— or —NH—(CHR_{c})ᵣ—CO—;
R₈ is a direct bond, —CO—(CH₂CH₂O)_{q}—(CH₂)ᵣ—NH— or —CO—(CHR_{c})ᵣ—NH—;
each R_{c} is chosen from —H, —CH₂—SO₃H or —SO₃H;
each q is an integer ranging from 1 to 24;
each r is an integer ranging from 1 to 8.

5. The conjugate according to claim 1, of formula (A): 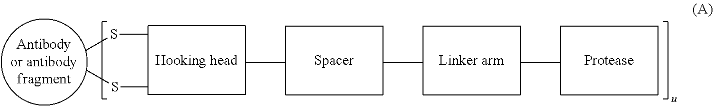
wherein u=1, 2, 3, 4, 5 or 6,
and wherein the linker arm is —CO—(CH₂)ₜ-R₉-, 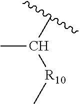
or —CH₂-R₁₀-;
t is an integer ranging from 1 to 10;
R₉ is -R₅-, —CO-R₇-NH—(CH₂CH₂O)_{q}—(CH₂)ᵣ-R₅-, —NH-R₈—CO—(CH₂CH₂O)_{q}—(CH₂)ᵣ-R₅-, —CO-R₇-NH—(CH₂)ᵣ-R₅-, —NH-R₈—CO—(CH₂)ᵣ-R₅-, —CONH—(CH₂CH₂O)_{q}—(CH₂)ᵣ—NHCO—(CH₂CH₂O)_{q}—(CH₂)ᵣ-R₅-; —CONH—(CHR_{c})ᵣ—NHCO—(CH₂CH₂O)_{q}—(CH₂)ᵣ-R₅-, —NHCO—(CH₂CH₂O)_{q}—(CH₂)ᵣ—CONH—(CH₂CH₂O)_{q}—(CH₂)ᵣ-R₅-; —NHCO—(CHR_{c})ᵣ—CONH—(CH₂CH₂O)_{q}—(CH₂)ᵣ-R₅-;
R₁₀ is a group of formula: 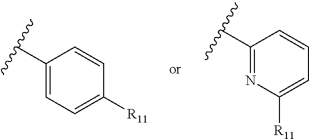
R₇ is a direct bond, —NH—(CH₂CH₂O)_{q}—(CH₂)ᵣ—CO— or —NH—(CHR_{c})ᵣ—CO—;
R₈ is a direct bond, —CO—(CH₂CH₂O)_{q}—(CH₂)ᵣ—NH— or —CO—(CHR_{c})ᵣ—NH—;
R₁₁ is —(CH₂)ᵣ-R₅-, —CONH—(CH₂)ᵣ-R₅-, —NHCO—(CH₂)ᵣ-R₅-, —NH—(CH₂)ᵣ-R₅-, —CONH—(CH₂CH₂O)_{q}—(CH₂)ᵣ-R₅-, —NHCO—(CH₂CH₂O)_{q}—(CH₂)ᵣ-R₅-;
R₅ has the definition given in claim 3;
each R_{c} is chosen from —H, —CH₂—SO₃H or —SO₃H;
each q is an integer ranging from 1 to 24;
each r is an integer ranging from 1 to 8.

6. The conjugate according to claim 1, of formula (A): 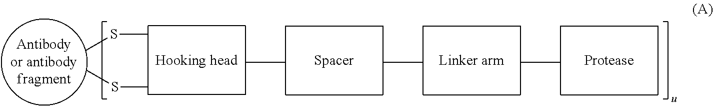
wherein u=1, 2, 3, 4, 5 or 6,
and wherein the hooking head is a compound of formula (I): 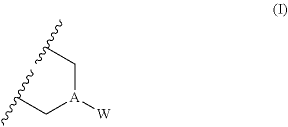
wherein:
A is the residue of a phenyl or a pyridyl;
W is —CONR₃R₄;
R₃ is —H, —(C₁-C₆)alkyl or —(CH₂)ᵥ—SO₃H;
R₄ is —(CH₂CH₂O)_{q}—(CH₂)ᵣ-R₅-, or —(CHR_{c})ᵣ-R₅-;
R₅ is chosen from: 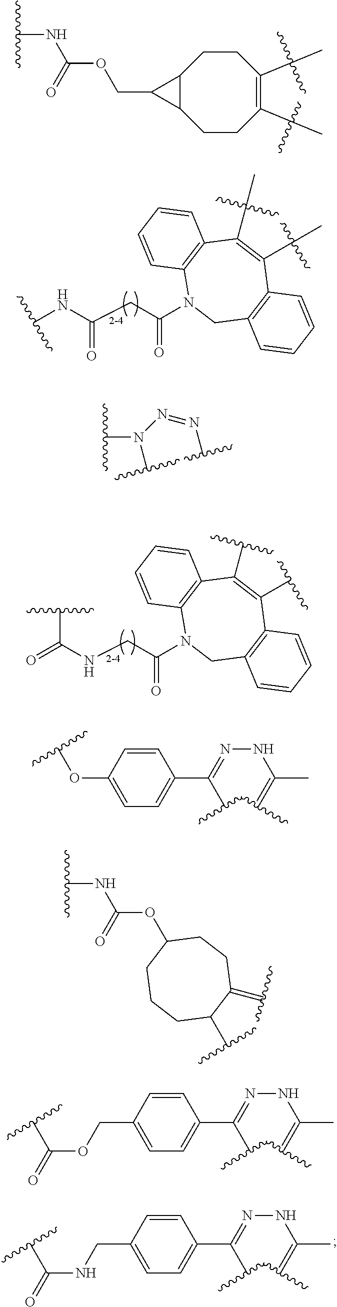
R_{c} is chosen from —H, —CH₂—SO₃H or —SO₃H;
q is an integer ranging from 1 to 12;
r is an integer ranging from 1 to 6;
v is an integer ranging from 1 to 6.

7. The conjugate according to claim 1, of formula (A); 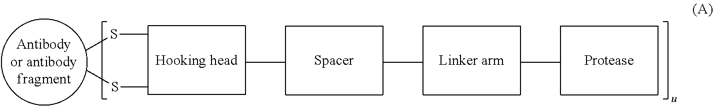
wherein u=1, 2, 3, 4, 5 or 6,
and wherein the hooking head is a compound of formula (Ib) or (Ib′): 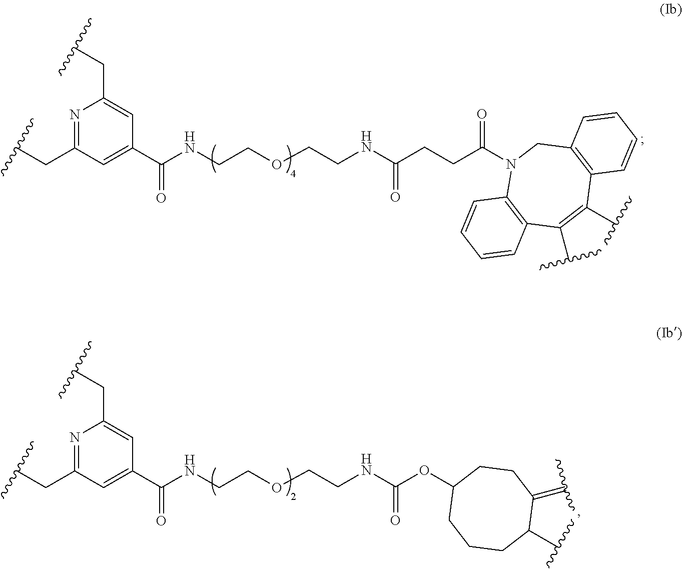
wherein:
A is the residue of a phenvl or a pyridyl;
W is —ORₐ, —COR₂, —CONR₃R₄ or —NR₃COR₄;
Rₐ is —(CH₂CH₂O)_{q}—(CH₂)ᵣR₅-, —(CHR_{c})ᵣ-R₅-, —COR_{b}, —(CHR_{c})ᵣ—NHCO—(CH₂CH₂O)—(CH₂)ᵣ- R₅- or —(CHR_{c})ᵣ—CONH—(CH₂CH₂O)_{q}—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R_{b} is —(CH₂CH₂O)_{q}—(CH₂)ᵣR₅-, —O(CH₂CH₂O)_{q}—(CH₂)ᵣR₅-, —(CHR_{c})ᵣR₅-, —O(CHR_{c})ᵣR₅-, —(CHR_{c})ᵣ—NHCO—(CH₂CH₂O)_{q}—(CH₂)ᵣ-R₅-, —(CHR_{c})rCONH—(CH₂CH₂O)_{q}—(CH₂)ᵣR₅-, —(CH₂CH₂O)_{q}—(CH₂)ᵣ—NHCO—(CHR_{c})ᵣR₅- or —(CH₂CH₂O)_{q}—(CH₂)ᵣ—CONH—(CHR_{c})ᵣ-R₅-;
R₂ is —(CH₂CH₂O)—(CH₂)-R₅-, —(CHR_{c})-R₅-, —O(CH₂CH₂O)—(CH₂)ᵣ-R₅-, —O(CHR_{c})-R₅-, —O(CHR_{c})ᵣ—NHCO—(CH₂CH₂O)—(CH₂)ᵣ-R₅-, —O(CHR_{c})ᵣ—CONH—(CH₂CH₂O)—(CH₂)ᵣ-R₅-, —O(CH₂CH₂O)—(CH₂)ᵣ—NHCO—(CR_{c}H)ᵣ-R₅— or —O(CH₂CH₂O)—(CH₂)ᵣ—CONH—(CHR_{c})-R₅-;
R₃ is —H, —(C₁-C₆)alkyl or —(CH₂)ᵥ—SO₃H:
R₄ is —(CH₂CH₂O)—(CH₂)ᵣ-R₅-, —(CHR_{c})ᵣ-R₅-, —(CHR_{c})ᵣ—NHCO—(CH₂CH₂O)—(CH₂)ᵣ-R₅-, —(CHR_{c})ᵣ—CONH—(CH₂CH₂O)—(CH₂)ᵣ-R₅-, —(CH₂CH₂O)—(CH₂)ᵣ—NHCO—(CHR_{c})-R₅— or —(CH₂CH₂O)—(CH₂)ᵣ—CONH—(CHR_{c})-R₅-;
R₅ is chosen from: 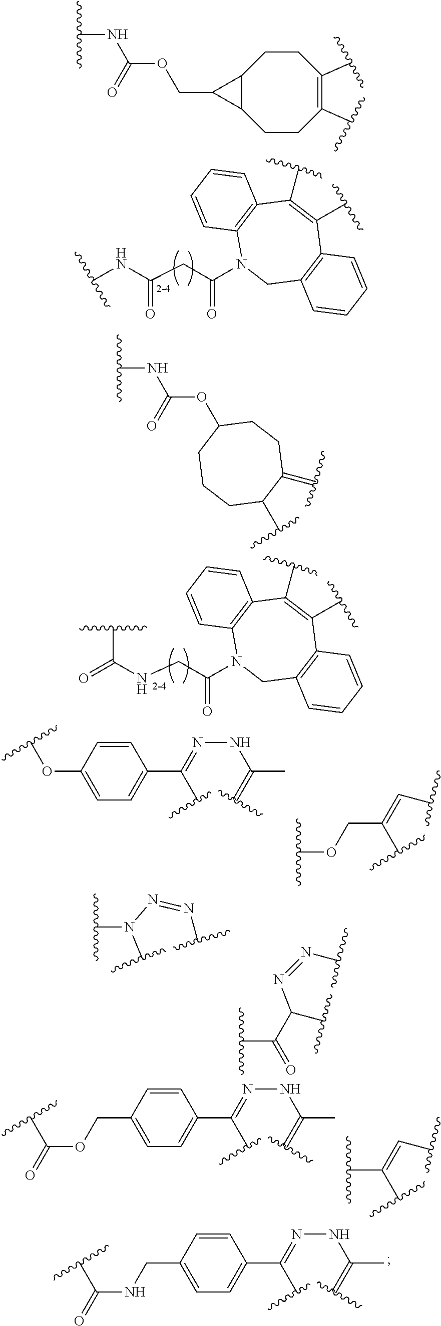
each R_{c} is chosen from —H, —CH₂—SO₃H or —SO₃H:
q is an integer ranging from 1 to 24;
each r is an integer ranging from 1 to 8;
each v is an integer ranging from 1 to 6.

8. The conjugate according to claim 1, of formula (B) or (C): 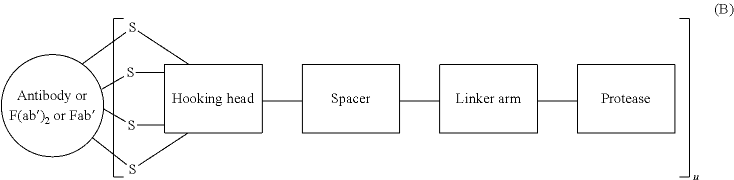
wherein u 1, 2 or 3;

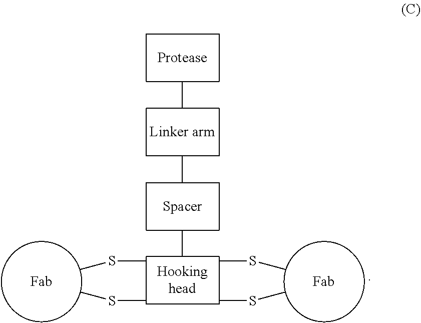

9. The conjugate according to claim 1, of formula (B) or (C): 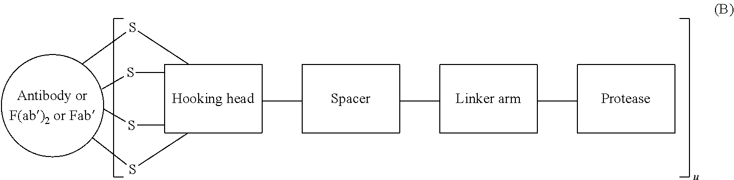
wherein u=1, 2 or 3; 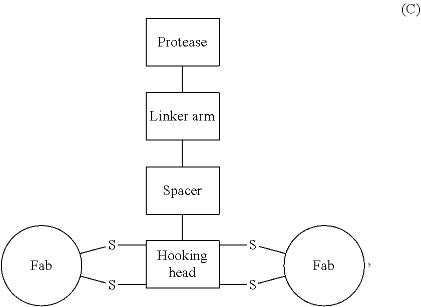
and wherein the hooking head is a compound of formula (II): 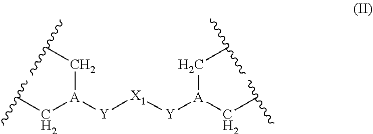
wherein:
each A is the residue of a phenyl or pyridyl;
each Y is a direct bond, —CH₂—, —O—, —S—, —CO—, —NH— or —C(═NR₁)—;
X₁ is chosen from: 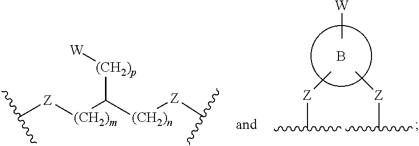 [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxy]methyl]-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)_{q}—(CH₂)ᵣ-R₅-, —(CHR_{c})ᵣ-R₅-, —COR_{b}, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣ-R₅— or —(CH₂CH₂O)_{q}—(CH₂)ᵣ—CONH—(CHR_{c})ᵣ-R₅-;
R_{b} is —(CH₂CH₂O)_{q}—(CH₂)ᵣ-R₅-, —O(CH₂CH₂O)_{q}—(CH₂)ᵣ-R₅-, —(CHR_{c})ᵣ-R₅-, —O(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣR₅- or —(CH₂CH₂O)_{q}—(CH₂)ᵣ—CONH—(CHR_{c})ᵣR₅-;
R₁ is —H, —OH or —(C₁-C₆)alkyl;
R₂ is —(CH₂CH₂O)_{q}—(CH₂)ᵣ-R₅-, —(CHR_{c})ᵣ-R₅-, —O(CH₂CH₂O)_{q}—(CH₂)ᵣ-R₅-, —O(CHR_{c})ᵣ-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)_{q}—(CH₂)ᵣ—NHCO—(CHR_{c})ᵣ-R₅— or —O(CH₂CH₂O)_{q}—(CH₂)ᵣ—CONH—(CHR_{c})ᵣ-R₅-;
R₃ is —H, —(C₁-C₆)alkyl or —(CH₂)ᵥ—SO₃H;
R₄ is —(CH₂CH₂O)_{q}—(CH₂)ᵣ-R₅-, —(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})ᵣ-R₅— or —(CH₂CH₂O)_{q}—(CH₂)ᵣ—CONH—(CHR_{c})ᵣR₅-;
R₅ is chosen from: 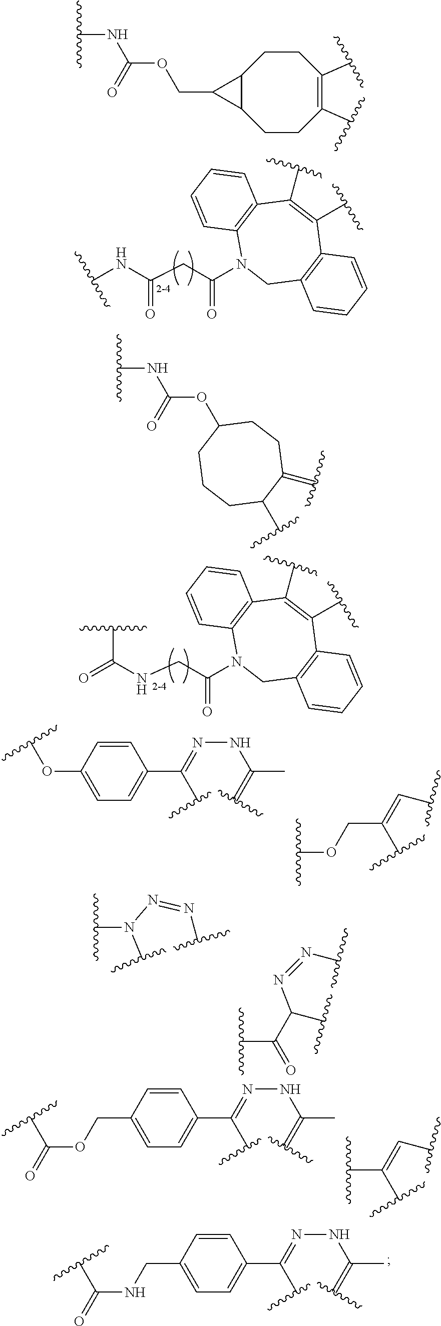
each R_{c} is independently —H, —CH₂—SO₃H or —SO₃H;
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
y is an integer ranging from 1 to 6.

10. The conjugate according to claim 1, of formula (B) or (C): 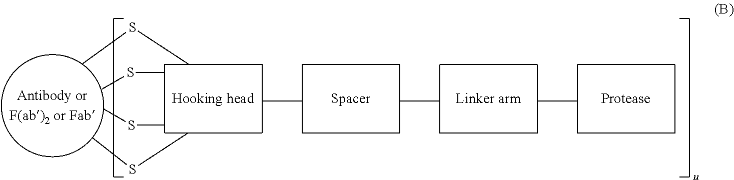
wherein u 1, 2 or 3 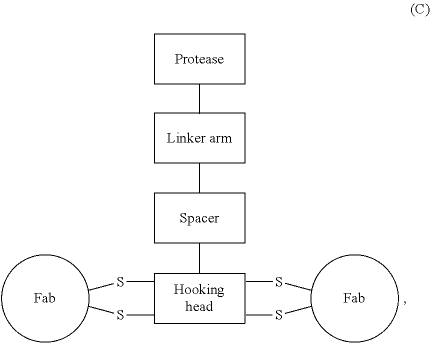
wherein the spacer is a direct bond or a group of formula -(R₅-R₆-R₅′)ₛ wherein:
R₅ and R₅′ are different from one another and are each chosen from: 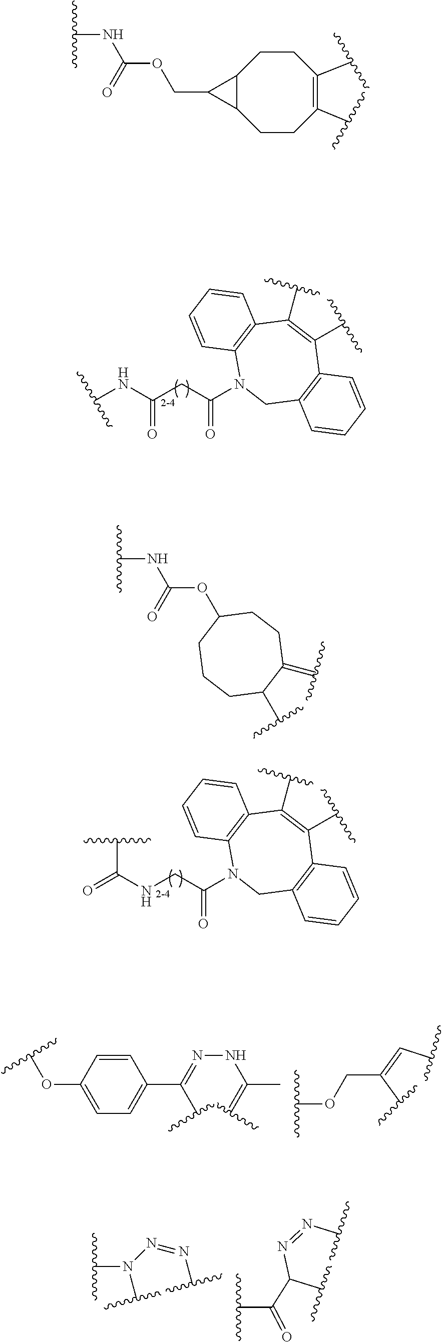 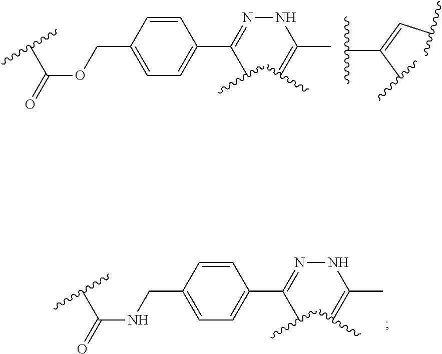
R₆ is —(CH₂CH₂O)—(CH₂)ᵣ, —(CH₂CH₂O)—(CH₂)ᵣ—CO-R₇-NH—(CH₂CH₂O)—(CH₂)ᵣ, —(CH₂CH₂O)_{q}—(CH₂)ᵣ—NH-R₈—CO—(CH₂CH₂O)_{q}—(CH₂)ᵣ—, —(CH₂)ᵣ—NHCO—(CHR_{c})ᵣ—, —(CH₂CH₂O)_{q}—(CH₂)ᵣ—CONH—(CH₂CH₂O)_{q}—(CH₂)ᵣ—NHCO—(CH₂CH₂O)_{q}—(CH₂)ᵣ—, —(CH₂CH₂O)_{q}—(CH₂)ᵣ—CONH—(CHR_{c})ᵣ—NHCO—(CH₂CH₂O)_{q}—(CH₂)ᵣ—, —(CH₂CH₂O)_{q}—(CH₂)ᵣ NHCO—(CH₂CH₂O)_{q}—(CH₂)ᵣ—CONH—(CH₂CH₂O)_{q}—(CH₂)ᵣ or —(CH₂CH₂O)_{q}—(CH₂)ᵣ—NHCO—(CHR_{c})ᵣ—CONH—(CH₂CH₂O)_{q}—(CH₂)ᵣ—;
R₇ is a direct bond, —NH—(CH₂CH₂O)_{q}—(CH₂)ᵣ—CO— or —NH—(CHR_{c})ᵣ—CO—;
R₈ is a direct bond, —CO—(CH₂CH₂O)_{q}—(CH₂)ᵣ—NH— or —CO—(CHR_{c})ᵣ—NH—;
each R_{c} is chosen from —H, —CH₂—SO₃H or —SO₃H;
each q is an integer ranging from 1 to 24;
each r is an integer ranging from 1 to 8;
s is an integer ranging from 1 to 10.

11. The conjugate according to claim 1, of formula (B) or (C): 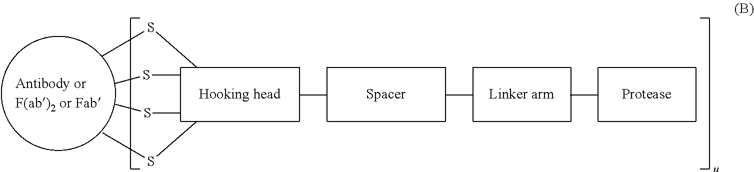
wherein u=1, 2 or 3; 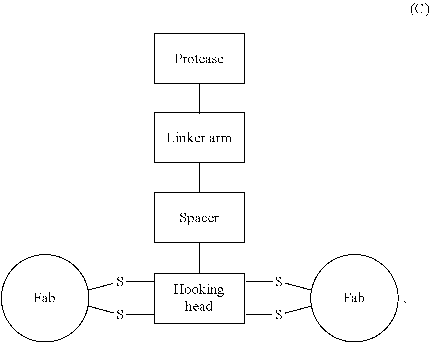
wherein the linker arm is —CO—(CH₂)ₜ-R₉—, 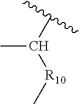
or —CH₂-R₁₀-, where:
t is an integer ranging from 1 to 10;
R₉ is -R₅-, —CO-R₇-NH—(CH₂CH₂O)_{q}—(CH₂)ᵣ-R₅-, —NH-R₈—CO—(CH₂CH₂O)_{q}—(CH₂)ᵣ-R₅-, —CO—R₇-NH—(CH₂)ᵣ-R₅-, —NH-R₈—CO—(CH₂)ᵣ-R₅-, —CONH—(CH₂CH₂O)_{q}—(CH₂)ᵣ—NHCO—(CH₂CH₂O)_{q}—(CH₂)ᵣ-R₅-; —CONH—(CHR_{c})ᵣ—NHCO—(CH₂CH₂O)_{q}—(CH₂)ᵣ-R₅-, —NHCO—(CH₂CH₂O)_{q}—(CH₂)ᵣ CONH—(CH₂CH₂O)_{q}—(CH₂)ᵣ-R₅-; —NHCO—(CHR_{c})ᵣ—CONH—(CH₂CH₂O)_{q}—(CH₂)ᵣ-R₅-;
R₁₀ is a group of formula: 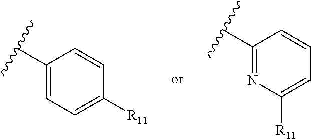
wherein
R₁₁ is —(CH₂)ᵣ-R₅-, —CONH—(CH₂)ᵣ-R₅-, —NHCO—(CH₂)ᵣ-R₅-, —NH—(CH₂)ᵣ-R₅-, —CONH—(CH₂CH₂O)_{q}—(CH₂)ᵣ-R₅-, —NHCO—(CH₂CH₂O)_{q}—(CH₂)ᵣ-R₅-.
R₇ is a direct bond, —NH—(CH₂CH₂O)_{q}—(CH₂)ᵣ—CO— or —NH—(CHR_{c})ᵣ—CO—;
R₈ is a direct bond, —CO—(CH₂CH₂O)_{q}—(CH₂)ᵣ—NH— or —CO—(CHR_{c})ᵣ—NH—;
R₅ has the definition given in claim 9;
each R_{c} is chosen from —H, —CH₂—SO₃H or —SO₃H;
each q is an integer ranging from 1 to 24;
each r is an integer ranging from 1 to 8.

12. The conjugate according to claim 1, of formula (B) or (C): 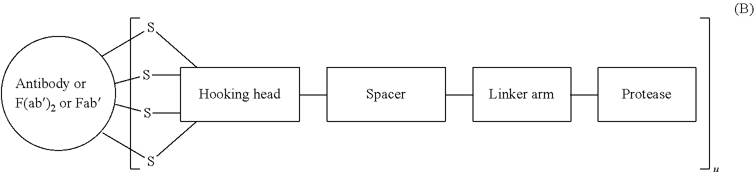
wherein u 1, 2 or 3 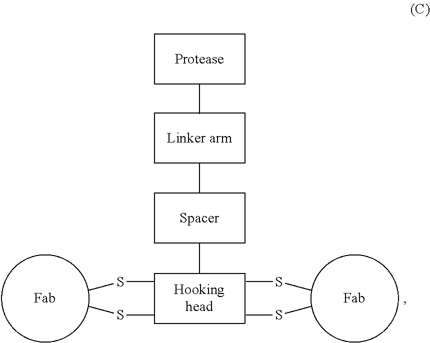
wherein the hooking head is a compound of formula (II): 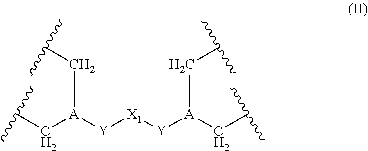
wherein:
each A is the residue of a pyridyl;
each Y is a direct bond, —CH₂—, —O—, —S—, —CO—, —NH— or —C(═NR₁)—;
X₁ is chosen from: 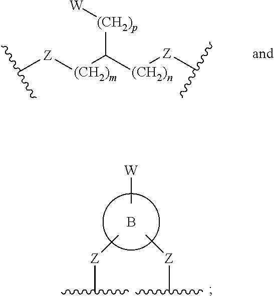 [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxy]methyl]-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)_{q}—(CH₂)ᵣR₅-, —(CHR_{c})ᵣ-R₅-, —COR_{b}, —(CHR_{c})ᵣ—NHCO—(CH₂CH₂O)—(CH₂)ᵣ- R₅-, —(CHR_{c})ᵣ—CONH—(CH₂CH₂O)_{q}—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R_{b} is —(CH₂CH₂O)_{q}—(CH₂)-R₅-, —O(CH₂CH₂O)_{q}—(CH₂)-R₅-, —(CHR_{c})-R₅-, —O(CHR_{c})-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)-R₅-, —(CH₂CH₂O)_{q}—(CH₂)ᵣ—NHCO—(CHR)ᵣ-R₅— or —(CH₂CH₂O)_{q}—(CH₂)ᵣ—CONH—(CHR_{c})ᵣ-R₅-;
R₁ is —H, —OH or —(C₁-C₆)alkyl;
R₂ is —(CH₂CH₂O)_{q}—(CH₂)ᵣ-R₅-, —(CHR_{c})-R₅-, —O(CH₂CH₂O)_{q}—(CH₂)ᵣ-R₅-, —O(CHR_{c})-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)_{q}—(CH₂)ᵣ—NHCO—(CHR_{c})-R₅— or —O(CH₂CH₂O)_{q}—(CH₂)ᵣ—CONH— (CHR)ᵣ-R₅-;
R₃ is —H, —(C₁-C₆)alkyl or —(CH₂)ᵥ—SO₃H:
R₄ is —(CH₂CH₂O)_{q}—(CH₂)-R₅-, —(CHR_{c})ᵣ-R₅-, —(CHR_{c})ᵣ—NHCO—(CH₂CH₂O)—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})-R₅— or —(CH₂CH₂O)_{q}—(CH₂)ᵣ—CONH—(CHR_{c})ᵣ-R₅-;
R₅ is chosen from: 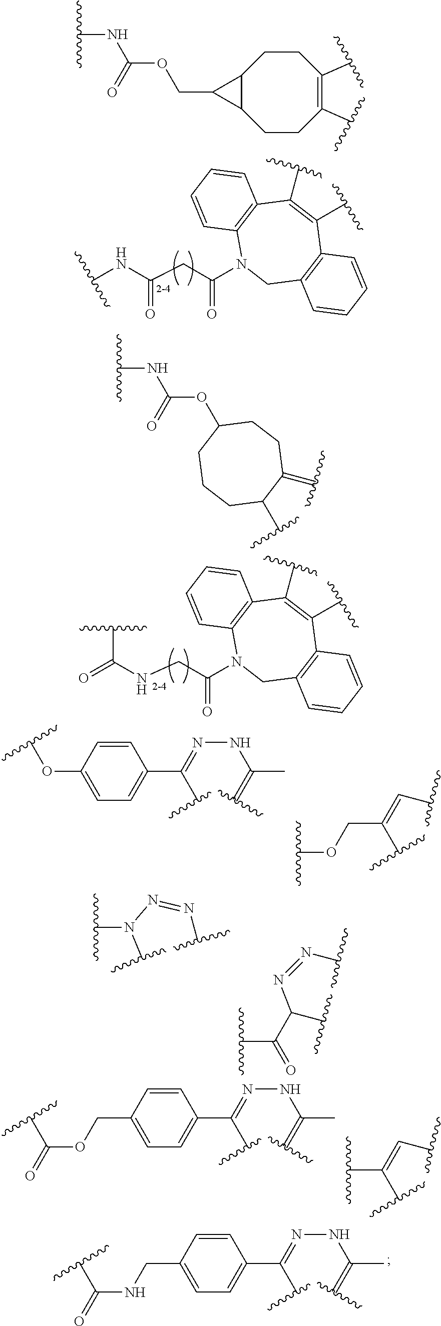
each R_{c} is independently —H, —CH₂—SO₃H or —SO₃H;
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
v is an integer ranging from 1 to 6.

13. The conjugate according to claim 1, of formula (B) or (C): 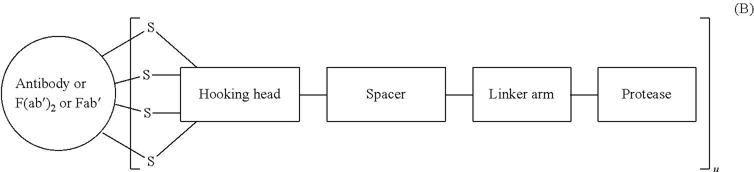
wherein u 1, 2 or 3 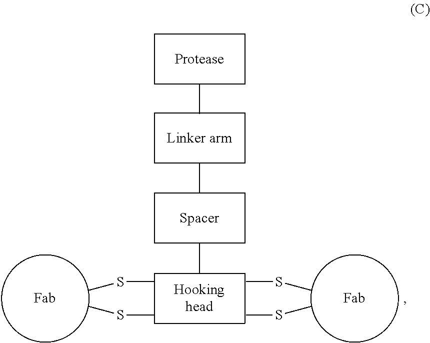
wherein the hooking head is a compound of formula (II): 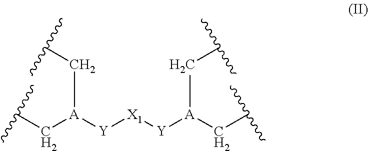
wherein:
each A is the residue of a phenyl or pyridyl;
each Y is chosen from a direct bond, —CO— and —NH—;
X₁ is chosen from: 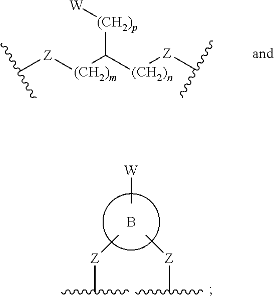 [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxylmethyll-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)(CH₂)ᵣR₅-, —(CHR_{c})ᵣ-R₅-, —COR_{b}, —(CHR_{c})ᵣ—NHCO—(CH₂CH₂O)—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})-R₅— or —(CH₂CH₂O)_{q}—(CH₂)ᵣ—CONH—(CHR_{c})ᵣ-R₅-;
R_{b} is —(CH₂CH₂O)_{q}—(CH₂)-R₅-, —O(CH₂CH₂O)_{q}—(CH₂)-R₅-, —(CHR_{c})-R₅-, —O(CHR_{c})-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)-R₅-, —(CH₂CH₂O)_{q}—(CH₂)ᵣ—NHCO—(CHR)ᵣ-R₅— or —(CH₂CH₂O)_{q}—(CH₂)ᵣ—CONH—(CHR_{c})ᵣ-R₅-;
R₁ is —H, —OH or —(C₁-C₆)alkyl;
R₂ is —(CH₂CH₂O)_{q}—(CH₂)ᵣ-R₅-, —(CHR_{c})-R₅-, —O(CH₂CH₂O)_{q}—(CH₂)ᵣ-R₅-, —O(CHR_{c})-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)_{q}—(CH₂)ᵣ—NHCO—(CHR_{c})-R₅— or —O(CH₂CH₂O)_{q}—(CH₂)ᵣ—CONH— (CHR)ᵣ-R₅-;
R₃ is —H, —(C₁-C₆)alkyl or —(CH₂)ᵥ—SO₃H:
R₄ is —(CH₂CH₂O)_{q}—(CH₂)ᵣ-R₅-, —(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})-R₅— or —(CH₂CH₂O)—(CH₂)ᵣ—CONH—(CHR_{c})ᵣ-R₅-;
R₅ is chosen from: 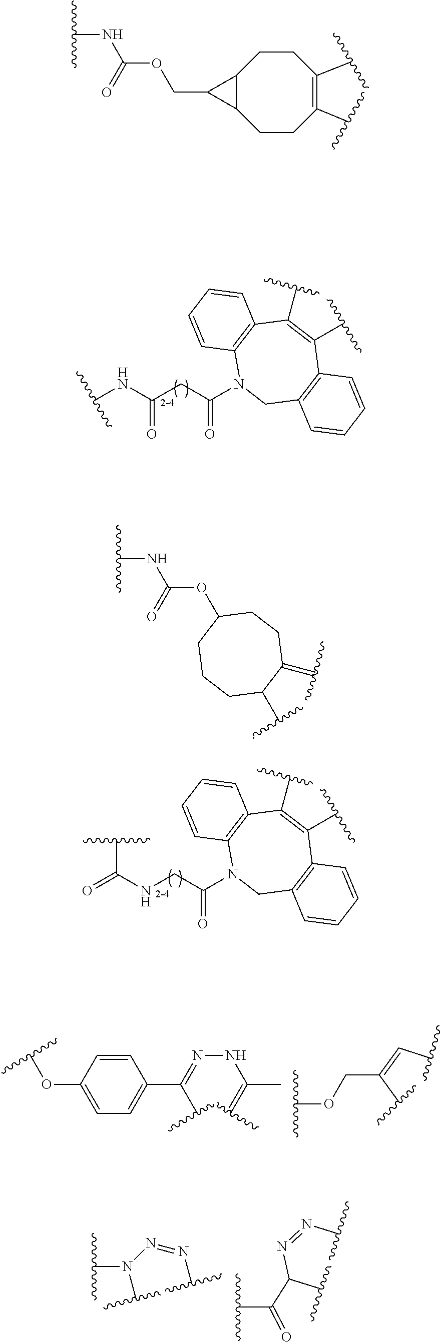 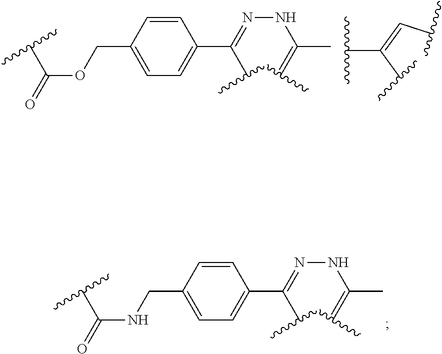
each R_{c} is independently —H, —CH₂—SO₃H or —SO₃H:
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
v is an integer ranging from 1 to 6.

14. The conjugate according to claim 1, of formula (B) or (C): 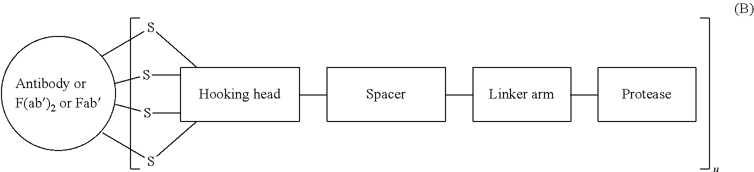
wherein u=1, 2 or 3; 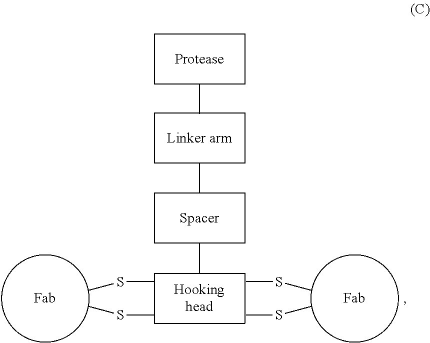
and wherein the hooking head is a compound of formula (II): 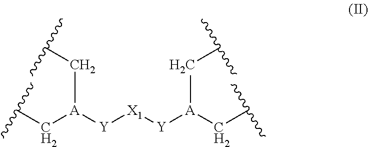
wherein:
each A is the residue of a phenyl or pyridyl;
each Y is a direct bond, —CH₂—, —O—, —S—, —CO—, —NH— or —C(═NR₁)—;
X₁ is chosen from: 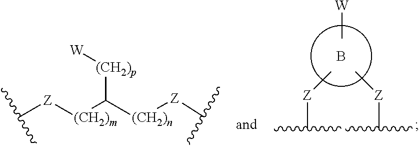 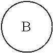
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxylmethyl]-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)_{q}—(CH₂)ᵣR₅-, —(CHR_{c})ᵣ-R₅-, —COR_{b}, —(CHR_{c})ᵣ—NHCO—(CH₂CH₂O)—(CH₂)ᵣ- R₅-, —(CHR_{c})ᵣ—CONH—(CH₂CH₂O)_{q}—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R_{b} is —(CH₂CH₂O)_{q}—(CH₂)ᵣR₅-, —O(CH₂CH₂O)—(CH₂)ᵣR₅-, —(CHR_{c})ᵣR₅-, —O(CHR_{c})ᵣR₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣR₅-, —(CH₂CH₂O) (CH₂)ᵣ—NHCO—(CHR_{c})ᵣR₅- or —(CH₂CH₂O)—(CH₂)ᵣ—CONH—(CHR_{c})ᵣR₅-;
R₁ is —H, —OH or —(C₁-C₆)alkyl;
R₂ is —(CH₂CH₂O)_{q}—(CH₂)ᵣR₅-, —(CHR_{c})ᵣR₅-, —O(CH₂CH₂O)_{q}—(CH₂)ᵣR₅-, —O(CHR_{c})ᵣ-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)—(CH₂)ᵣ—NHCO—(CHR_{c})ᵣ-R₅— or —O(CH₂CH₂O)—(CH₂)ᵣ—CONH— (CHR_{c})ᵣR₅-;
R₃ is —H, —(C₁-C₆)alkyl or —(CH₂)ᵥ—SO₃H:
R₄ is —(CH₂CH₂O)_{q}—(CH₂)ᵣR₅-, —(CHR_{c})ᵣ-R₅-, —(CHR_{c})ᵣ—NHCO—(CH₂CH₂O) (CH₂)ᵣ-R₅-, —(CHR_{c})ᵣ—CONH—(CH₂CH₂O)—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R₅ is chosen from: 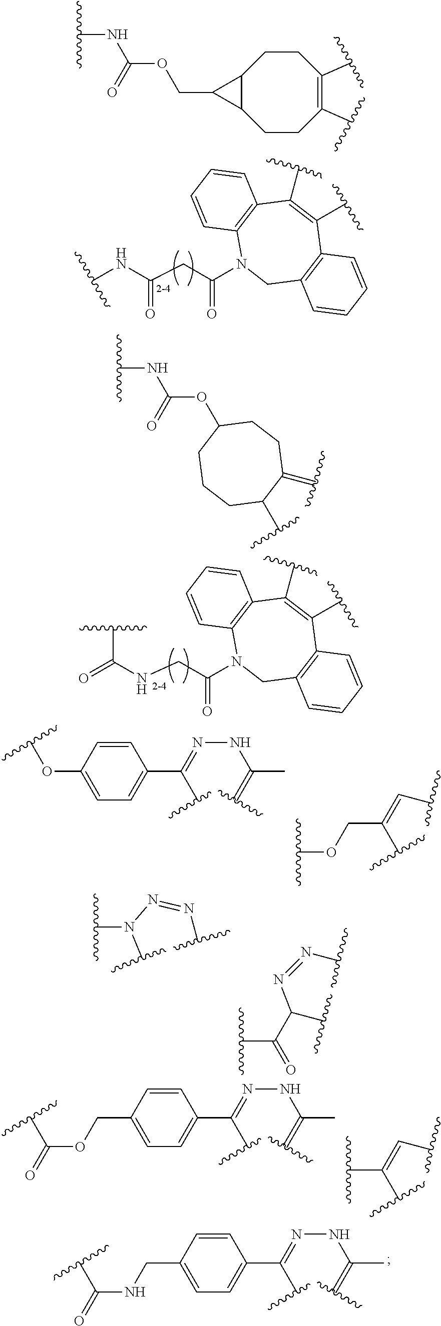
each R_{c} is independently —H, —CH₂—SO₃H or —SO₃H:
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
v is an integer ranging from 1 to 6,
wherein one of the groups Y and Z is —CO— and the other is —NH—.

15. The conjugate according to claim 1, of formula (B) or (C): 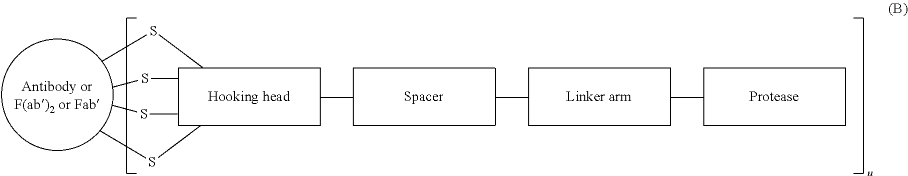
wherein u=1, 2 or 3 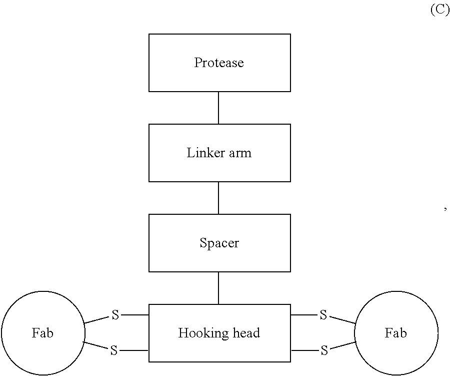
and wherein the hooking head is a compound of formula (II): 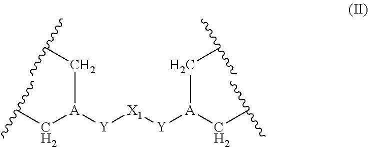
wherein:
each A is the residue of a phenyl or pyridyl;
each Y is a direct bond, —CH₂—, —O—, —S—, —CO—, —NH— or —C(═NR₁)—;
X₁ is of formula: 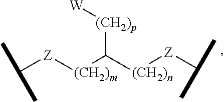
wherein
Z is —CO— or —NH—;
m and n are each, independently of one another, an integer ranging from 0 to 3;
p is equal to 0, 1 or 2;
W is —CONR₃R₄;
R₃ is —H or —(C₁-C₆)alkyl;
R₄ is —(CH₂CH₂O)_{q}—(CH₂)ᵣ-R₅-, or —(CHR_{c})ᵣ-R₅-;
R₅ is chosen from: 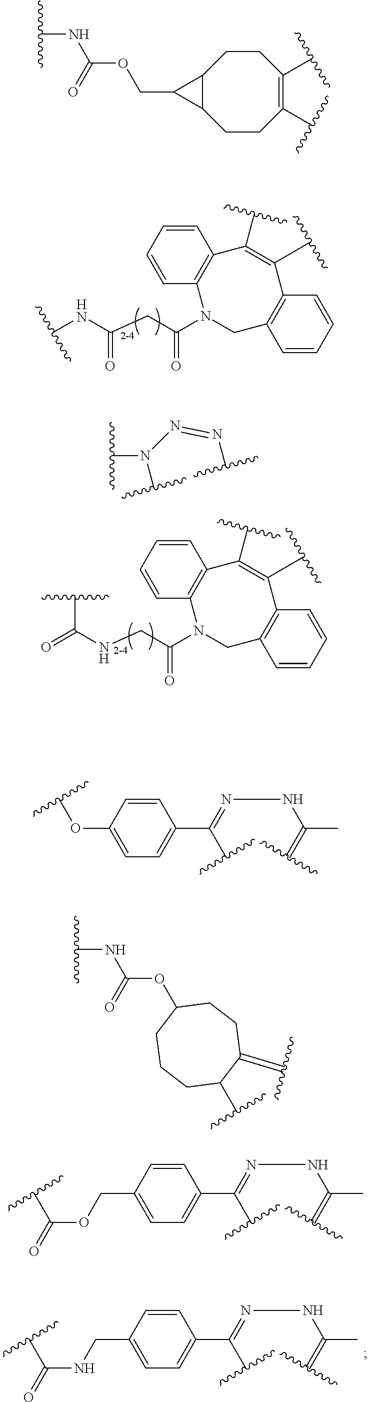
R_{c} is chosen from —H, —CH₂—SO₃H or —SO₃H;
q is an integer ranging from 1 to 12;
r is an integer ranging from 1 to 6; [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each g is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxylmethyl]-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)—(CH₂)ᵣR₅-, —(CHR_{c})ᵣ-R₅-, —COR_{b}, —(CHR_{c})ᵣ—NHCO—(CH₂CH₂O)—(CH₂)ᵣ-R₅-, —(CHR_{c})ᵣ—CONH—(CH₂CH₂O)—(CH₂)ᵣ-R₅-, —(CH₂CH₂O)_{c}—(CH₂)ᵣ—NHCO—(CHR_{c})ᵣ-R₅- or —(CH₂CH₂O)_{c}—(CH₂)ᵣ—CONH—(CHR_{c})ᵣ-R₅-;
R_{b} is —(CH₂CH₂O)ₜ—(CH₂)ᵣR₅-, —O(CH₂CH₂O)ₜ—(CH₂)ᵣR₅-, —(CHR_{c})-R₅-, —O(CHR_{c})ᵣ-R₅-, —(CHR_{c})ᵣ—NHCO—(CH₂CH₂O)ₜ—(CH₂)ᵣ-R₅-, —(CHR_{c})ᵣ—CONH—(CH₂CH₂O)—(CH₂-R₅-, —(CH₂CH₂O)_{c}—(CH₂)ᵣ—NHCO—(CHR_{c})ᵣ-R₅- or —(CH₂CH₂O)—(CH₂)ᵣ—CONH—(CHR_{c})ᵣ-R₅-;
R₁ is —H, —OH or —(C₁-C₆)alkyl;
R₂ is —(CH₂CH₂O)ₜ—(CH₂)ᵣR₅-, —(CHR)ᵣ-R₅-, —O(CH₂CH₂O)—(CH₂)rR₅-, —O(CHR_{c})ᵣ-R₅-, —O(CHR_{c})ᵣ—NHCO—(CH₂CH₂O)ₜ—(CH₂)ᵣ-R₅-, —O(CHR_{c})ᵣ—CONH—(CH₂CH₂O)_{q}—(CH₂)ᵣ-R₅-, —O(CH₂CH₂O)—(CH₂)ᵣ—NHCO—(CHR_{c})ᵣ-R₅- or —O(CH₂CH₂O)—(CH₂)ᵣ—CONH— (CHR_{c})-R₅-;
R₃ is —H, —(C₁-C₆)alkyl or —(CH₂)ᵥ—SO₃H:
R₄ is —(CH₂CH₂O)ₜ—(CH₂)ᵣR₅-, —(CHR_{c})ᵣ-R₅-, —(CHR_{c})ᵣ—NHCO—(CH₂CH₂O)_{c}—(CH₂)ᵣ-R₅- , —(CHR_{c})ᵣ—CONH—(CH₂CH₂O)_{c}—(CH₂)ᵣ-R₅-, —(CH₂CH₂O)_{c}—(CH₂)ᵣ—NHCO—(CHR_{c})ᵣ-R₅- or —(CH₂CH₂O)—(CH₂)ᵣ—CONH—(CHR_{c})ᵣR₅-;
R₅ is chosen from: 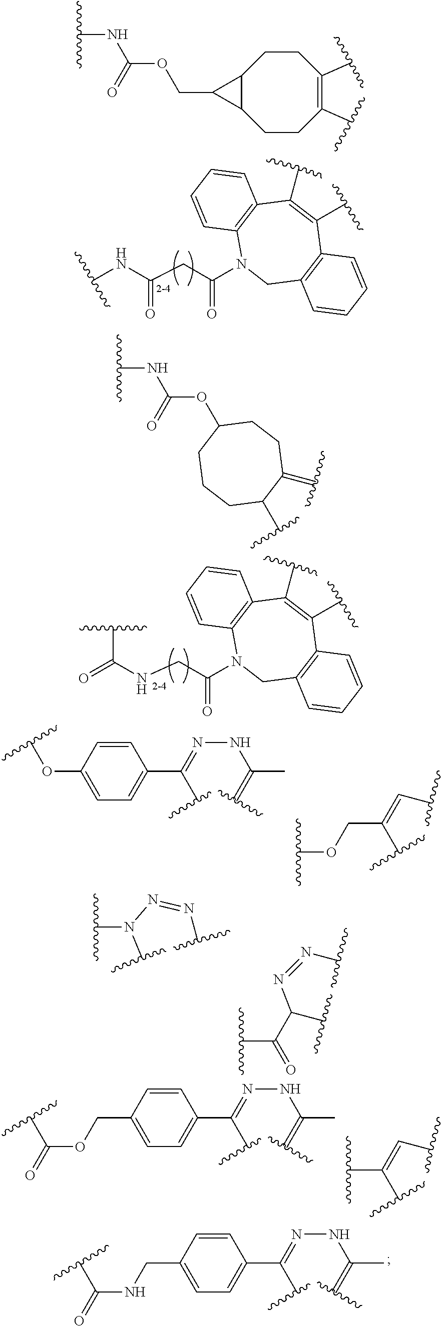
each R_{c} is independently —H, —CH₂—SO₃H or —SO₃H:
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
v is an integer ranging from 1 to 6.

16. The conjugate according to claim 1, of formula (B) or (C): 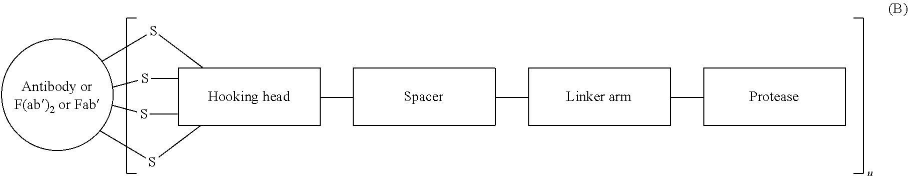
wherein u=1, 2 or 3; 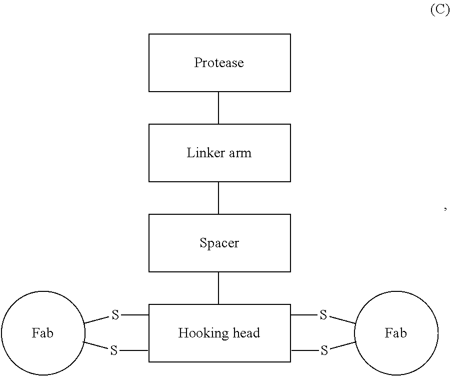
and wherein the hooking head is a compound of formula (II): 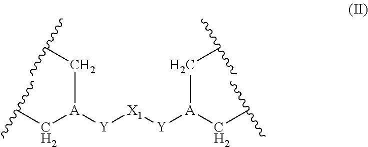
wherein:
each A is the residue of a phenyl or pyridyl;
each Y is a direct bond, —CH₂—, —O—, —S—, —CO—, —NH— or —C(═NR₁)—;
X₁ is a group:
chosen from: 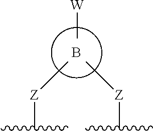 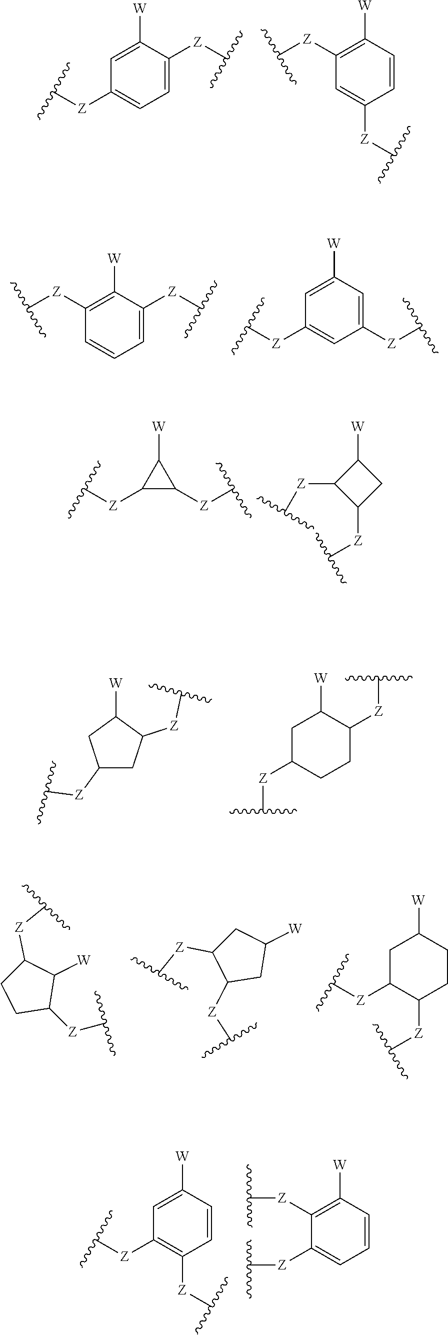 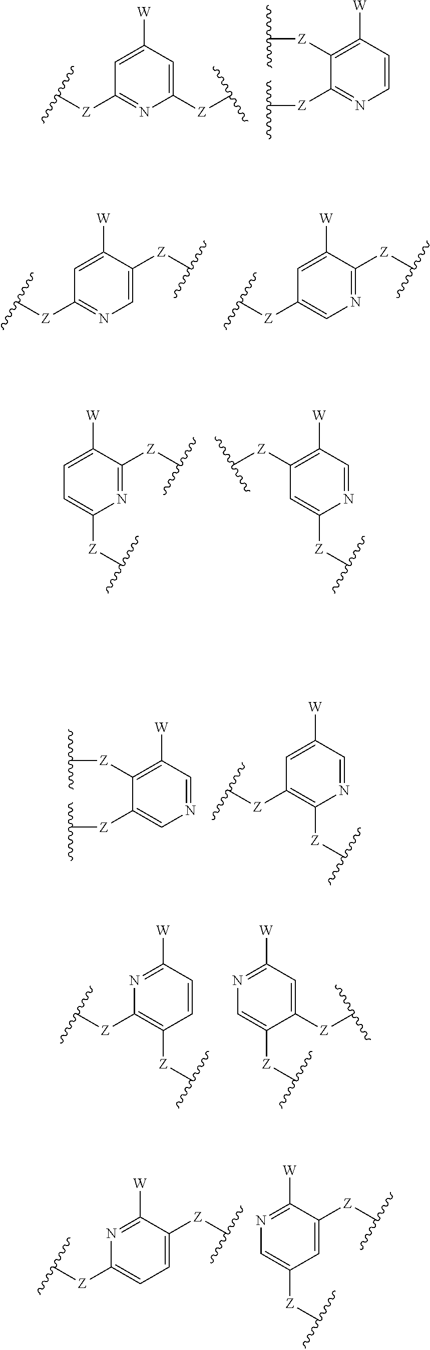 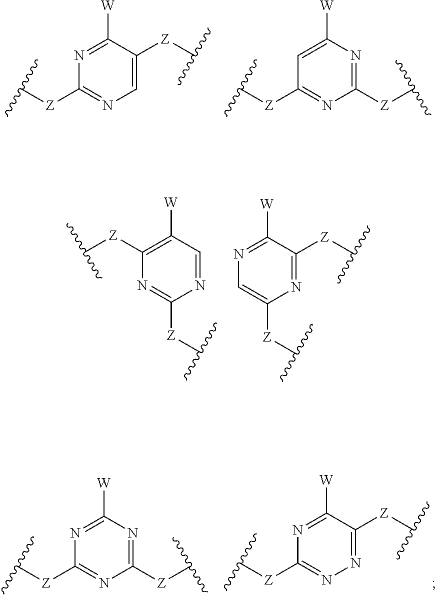
Z is —CO— or —NH—;
W is —CONR₃R₄;
R₃ is —H or —(C₁-C₆)alkyl;
R₄ is —(CHR_{c})ᵣ-R₅-, or —(CH₂CH₂O)_{q}—(CH₂)ᵣ-R₅-;
R₅ is chosen from: 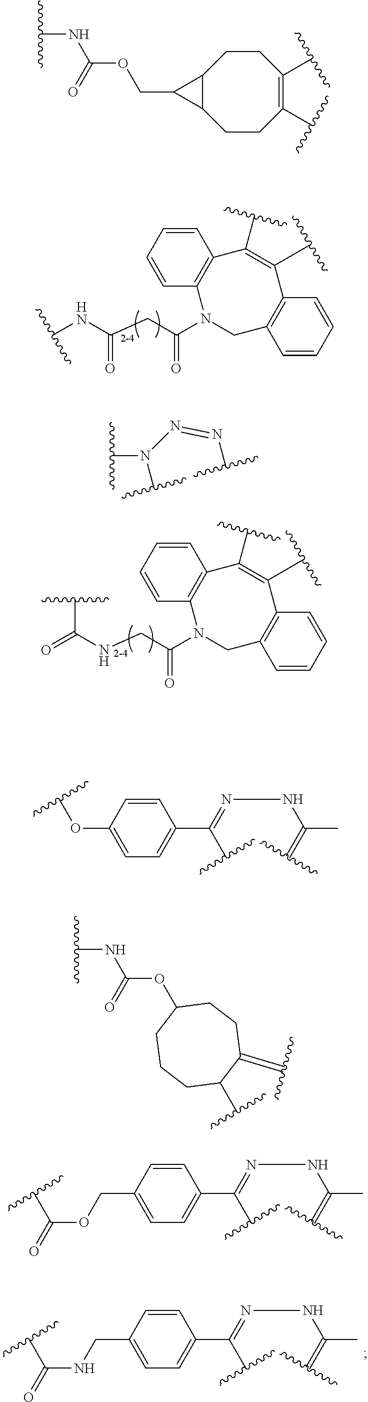
R_{c} is chosen from —H, —CH₂—SO₃H or —SO₃H;
q is an integer ranging from 1 to 12;
r is an integer ranging from 1 to 6; [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each g is an integer ranging from 1 to 24;
each r is an integer ranging from 1 to 8;
each s is an integer ranging from 0 to 6;
each s is an integer ranging from 1 to 6;
with the exception of the following compounds:
2,6-bis[2,6-bis(bromomethyl)phenyl]benzoic acid, and
1,3-bis[[3,5-bis(bromomethyl)phenyl]methyl]-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)_{q}—(CH₂)ᵣ-R₅-, —(CHR_{c})ᵣ-R₅-, —COR_{b}, —(CHR_{c})ᵣ—NHCO—(CH₂CH₂O)—(CH₂)ᵣ- R₅-, —(CHR_{c})ᵣ—CONH—(H₂CH₂O)_{q}—(CH₂)ᵣ-R₅-, —(CH₂)ᵣ-R₅-, —(CHR_{c})ᵣ-R₅- or —(CH₂CH₂O)C—(CH₂)ᵣ—CO_{N}H—(CHR_{c})ᵣ-R₅-;
R_{b} is —(CH₂CH₂O)_{q}—(CH₂)ᵣ-R₅-, —O(CH₂CH₂)ᵣ-R₅-, —(CH₂)ᵣ-R₅-, —(CHR_{c})-R₅-, —O(CHR_{c})ᵣ-R₅-, —(CHR_{c})ᵣ—NHCO—(H₂CCH₂O)_{q}—(CH₂)ᵣ-R₅-, —(CHR_{c})ᵣ—CONH—(CH₂CH₂O)_{q}—(CH₂)ᵣR₅-, —(CH₂CH₂O)_{q}—(CH₂)ᵣ—NHCO—(CHR_{c})ᵣ-R₅- or —(CH₂CH₂O)_{q}—(CH₂)ᵣ—CONH—(CHR_{c})ᵣ-R₅-;
R₁ is —H, —OH or —(C₁-C₆)alkyl:
R₂ is —(CH₂CH₂O)_{q}—(CH₂)ᵣR₅-, —(CHR_{c})ᵣ-R₅-, —O(CH₂CH₂O)_{q}—(CH₂)ᵣ-R₅-, —O(CHR_{c})ᵣ-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)—(CH₂)ᵣ—NHCO—(CHR_{c})ᵣ-R₅- or —O(CH₂CH₂O)—(CH₂)ᵣ—CONH— (CHR_{c})-R₅-;
R₃ is —H, —(C₁-C₆)alkyl or —(CH₂)ᵥ—SO₃H;
R₄ is —(CH₂CH₂O)_{q}—(CH₂)ᵣ-R₅-, —(CHR_{c})ᵣ-R₅-, —(CHR_{c})ᵣ—NHCO—(CH₂CH₂O) (CH₂)ᵣ-R₅-, —(CHR_{c})ᵣ—CONH—(CH₂CH₂O)—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R₅ is chosen from: 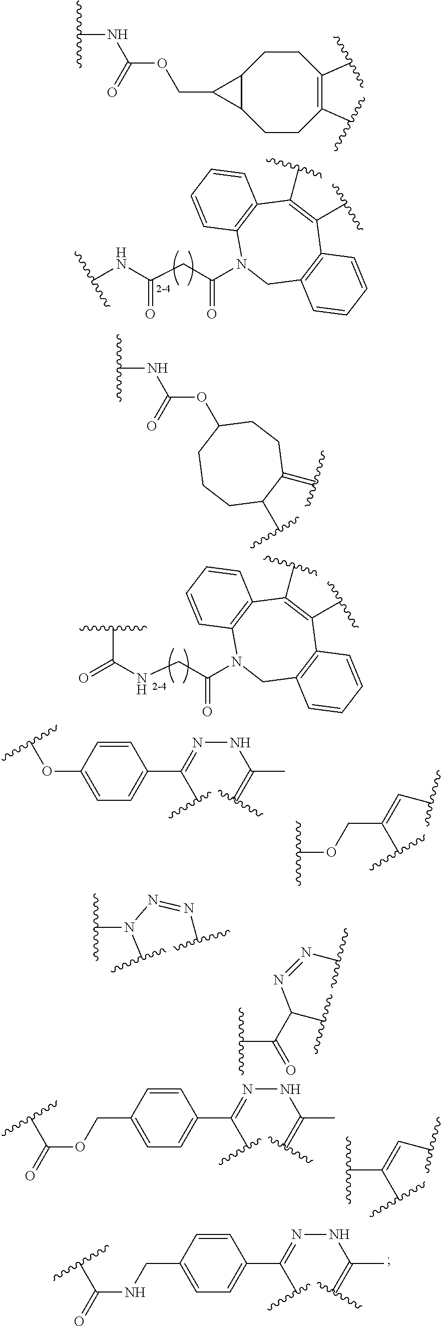
each R_{c} is independently —H, —CH₂—SO₃H or —SO₃H:
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
v is an integer ranging from 1 to 6.

17. The conjugate according to claim 1, of formula (B) or (C): 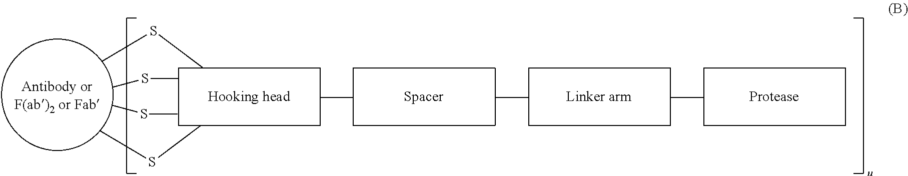
wherein u=1, 2 or 3 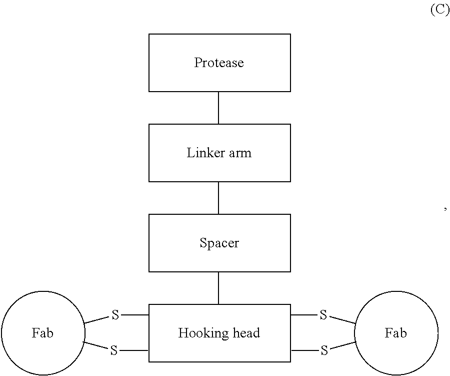
and wherein the hooking head is a compound of formula (II): 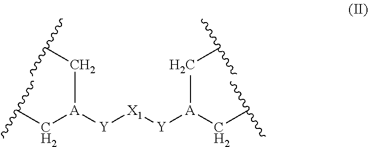
wherein:
each A is the residue of a phenyl or pyridyl;
each Y is a direct bond, —CH₂—, —O—, —S—, —CO—, —NH— or —C(═NR₁)—;
X₁ is chosen from: 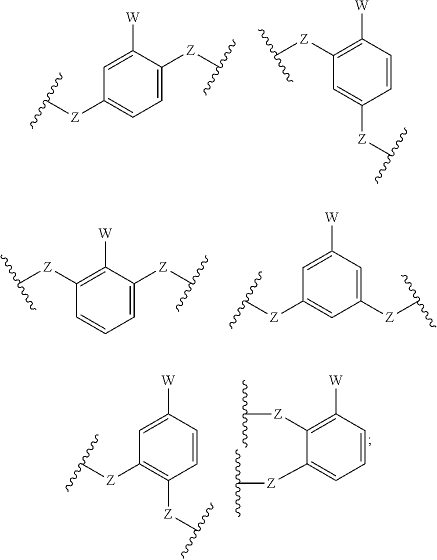 [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxylmethyl]-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)_{q}—(CH₂)ᵣR₅-, —(CHR_{c})ᵣ-R₅-, —COR_{b}, —(CHR_{c})ᵣ—NHCO—(CH₂CH₂O)—(CH₂)ᵣ- R₅-, —(CHR_{c})ᵣ—CONH—(CH₂CH₂O)_{q}—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R_{b} is —(CH₂CH₂O)_{q}—(CH₂)ᵣR₅-, —O(CH₂CH₂O)—(CH₂)ᵣR₅-, —(CHR_{c})ᵣR₅-, —O(CHR_{c})ᵣR₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣR₅-, —(CH₂CH₂O) (CH₂)ᵣ—NHCO—(CHR_{c})ᵣR₅- or —(CH₂CH₂O)—(CH₂)ᵣ—CONH—(CHR_{c})ᵣ-R₅-;
R₁ is —H, —OH or —(C₁-C₆)alkyl;
R₂ is —(CH₂CH₂O)_{q}—(CH₂)ᵣ-R₅-, —(CHR_{c})-R₅-, —O(CH₂CH₂O)_{q}—(CH₂)ᵣ-R₅-, —O(CHR_{c})-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)_{q}—(CH₂)ᵣ—NHCO—(CHR_{c})-R₅— or —O(CH₂CH₂O)_{q}—(CH₂)ᵣ—CONH— (CHR)ᵣ-R₅-;
R₃ is —H, —(C₁-C₆)alkyl or —(CH₂)ᵥ—SO₃H:
R₄ is —(CH₂CH₂O)_{q}—(CH₂)-R₅-, —(CHR_{c})ᵣ-R₅-, —(CHR_{c})ᵣ—NHCO—(CH₂CH₂O)—(CH₂)ᵣ-R₅-, —(CHR_{c})ᵣ—CONH—(CH₂CH₂O)_{q}—(CH₂)ᵣ-R₅-, —(CH₂CH₂O)_{q}—(CH₂)ᵣ—NHCO—(CHR_{c})-R₅— or —(CH₂CH₂O)—(CH₂)ᵣ—CONH—(CHR_{c})ᵣ-R₅-;
R₅ is chosen from: 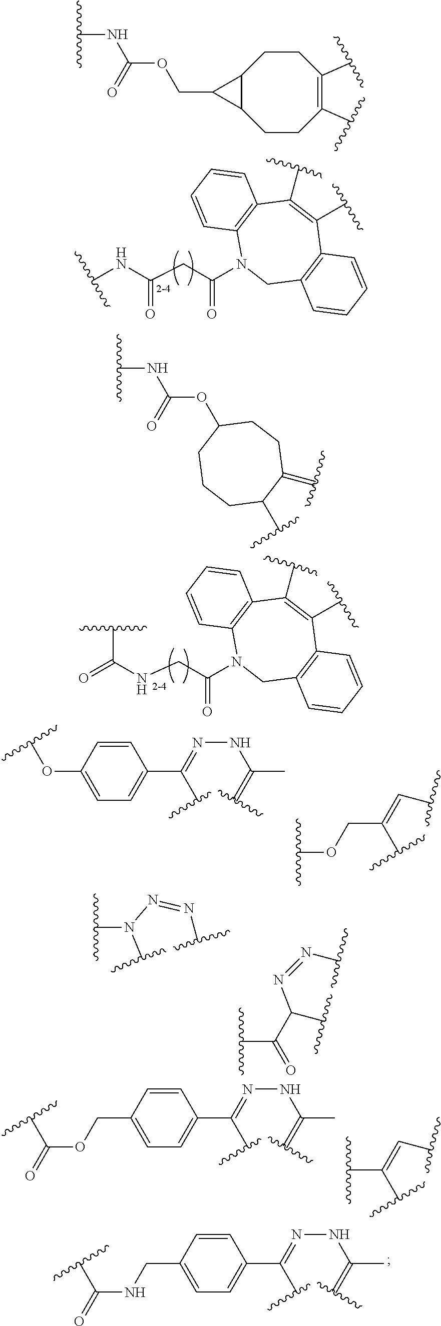
each R_{c} is independently —H, —CH₂—SO₃H or —SO₃H;
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
v is an integer ranging from 1 to 6.

18. The conjugate according to claim 1, of formula (B) or (C): 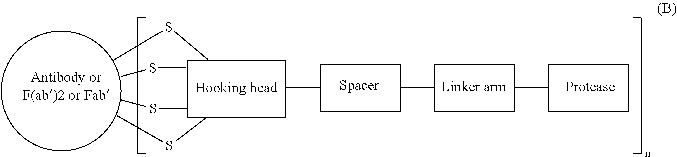
wherein u=1, 2 or 3 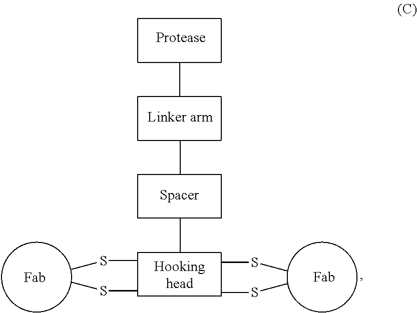
and wherein the hooking head is a compound of formula (II): 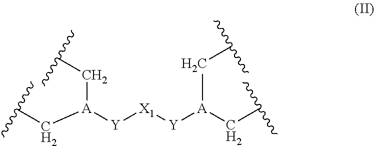
wherein:
each A is the residue of a phenyl or pyridyl;
each Y is a direct bond, —CH₂—, —O—, —S—, —CO—, —NH— or —C(═NR₁)—;
X₁ is chosen from: 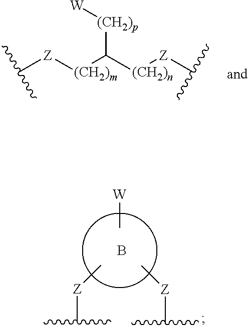 [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxylmethyl]-5-prop-2-ynoxy-benzene;
each Z is a direct bond, —CH₂—, —O—, —S—, —CO—, —NH— or —C(═NR₁)—;
wherein W is —CONR₃R₄;
R₃ is —H or —(C₁-C₆)alkyl;
R₄ is —(CH₂CH₂O)_{q}—(CH₂)ᵣ-R₅-, or —(CHR_{c})ᵣ-R₅-;
R₅ is chosen from: 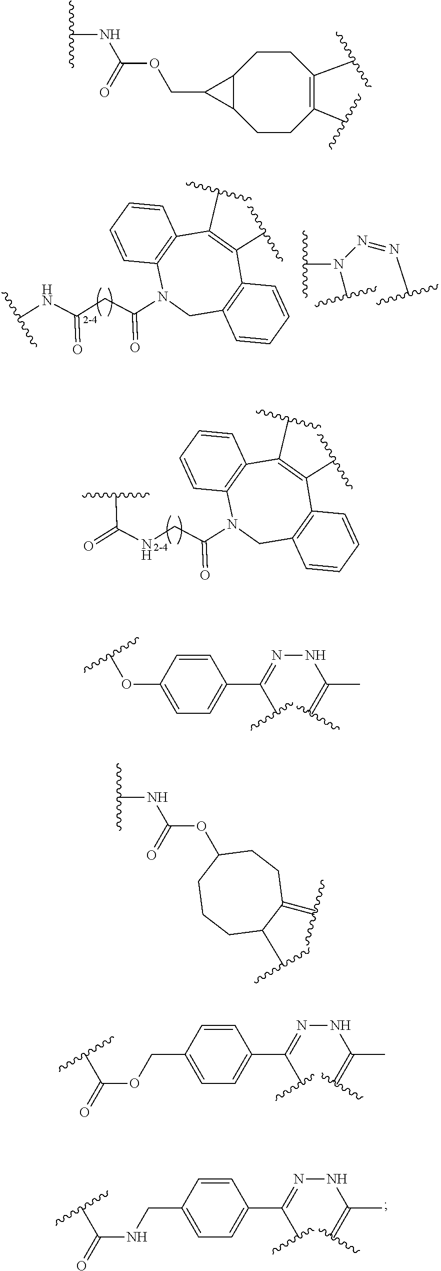
R_{c} is chosen from —H, —CH₂—SO₃H or —SO₃H;
m and n are each, independently of one another, an integer ranging from 0 to 3;
p is equal to 0, 1 or 2;
q is an integer ranging from 1 to 12;
r is an integer ranging from 1 to 6.

19. The conjugate according to claim 1, of formula (B) or (C): 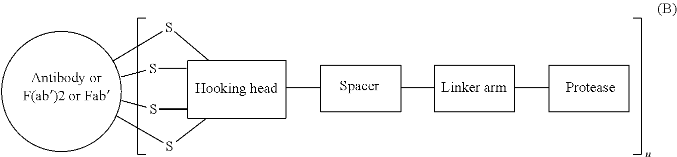
wherein u=1, 2 or 3 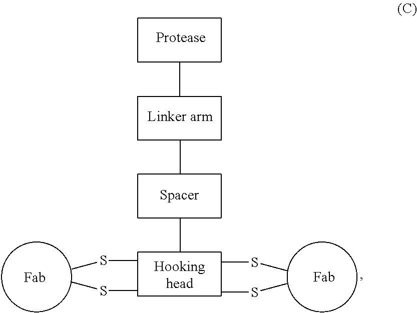
and wherein the hooking head is a compound of formula (IIa), (IIa′), (IIb), (IIb′), (IIc) or (IIc′):

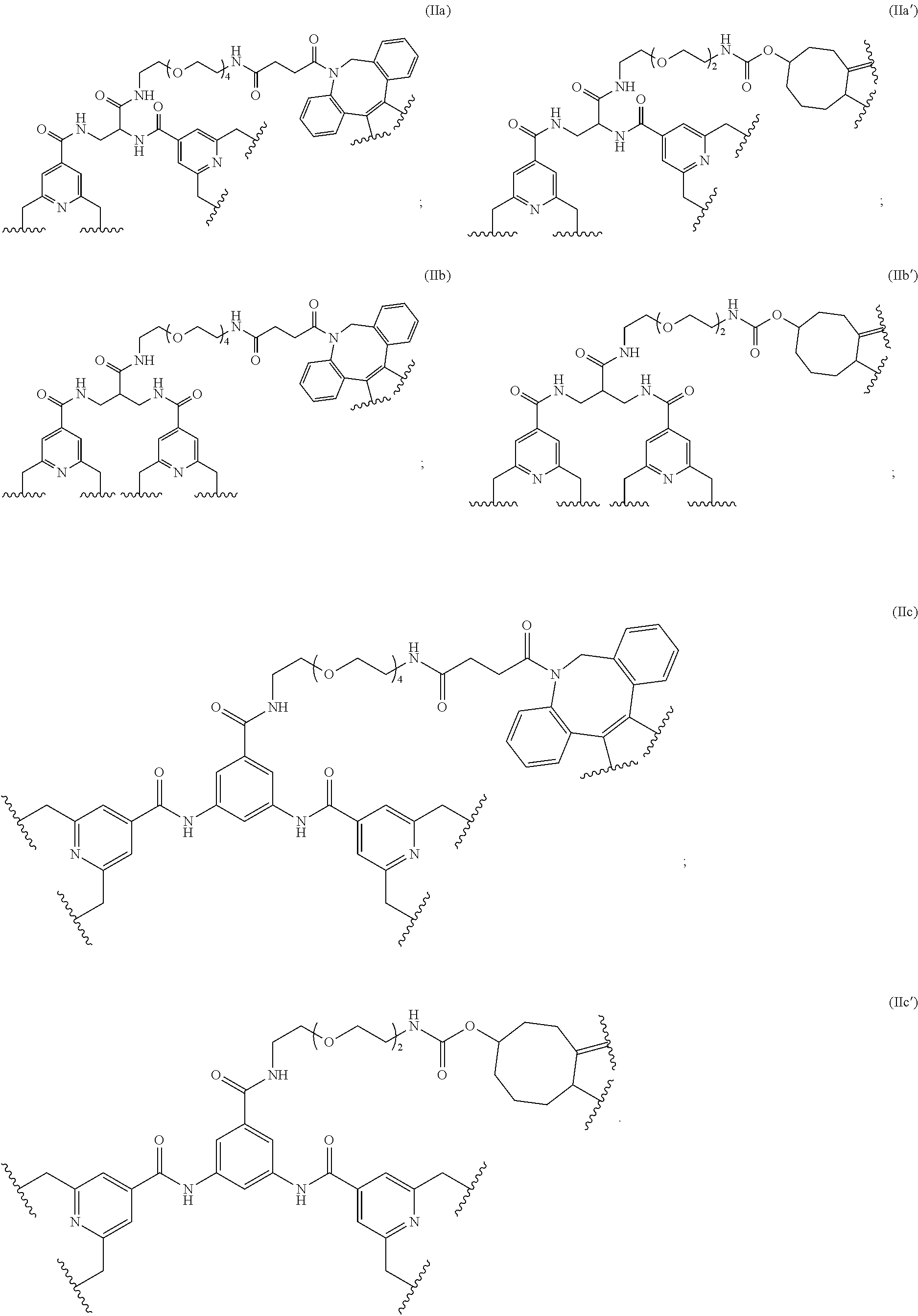

20. The conjugate according to claim 1, of formula (A): 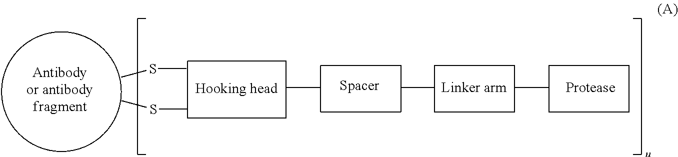
wherein u=1, 2, 3, 4, 5 or 6,
and wherein the hooking head is a compound of formula (I): 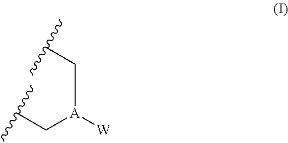
wherein:
A is the residue of a phenyl or a pyridyl;
W is —ORₐ, —COR₂, —CONR₃R₄ or —NR₃COR₄;
Rₐ is —(CH₂CH₂O)_{q}—(CH₂)ᵣ-R₅-, —(CHR_{c})ᵣ-R₅-, —COR_{b}, —(CHR_{c})ᵣ—NHCO—(CH₂CH₂O)—(CH₂)ᵣ- R₅-, —(CHR_{c})ᵣ—CONH—(CH₂CH₂O)_{q}—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R_{b} is —(CH₂CH₂O)_{q}—(CH₂)ᵣ-R₅-, —O(CH₂CH₂O)—(CH₂)ᵣ-R₅-, —(CHR_{c})ᵣ-R₅-, —O(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O) (CH₂)ᵣ—NHCO—(CHR_{c})ᵣR₅- or —(CH₂CH₂O)—(CH₂)ᵣ—CONH—(CHR_{c})ᵣR₅-;
R₂ is —(CH₂CH₂O)_{q}—(CH₂)ᵣ-R₅-, —(CHR_{c})ᵣ-R₅-, —O(CH₂CH₂O)_{q}—(CH₂)ᵣ-R₅-, —O(CHR_{c})ᵣ- R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)_{q}—(CH₂)ᵣ—NHCO—(CR_{c}H)ᵣ-R₅— or —O(CH₂CH₂O) (CH₂)ᵣ—CONH—(CHR_{c})ᵣ-R₅-;
R₃ is —H, —(C₁-C₆)alkyl or —(CH₂)ᵥ—SO₃H:
R₄ is —(CH₂CH₂O)_{q}—(CH₂)ᵣ-R₅-, —(CHR_{c})ᵣ-R₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R₅ is chosen from: 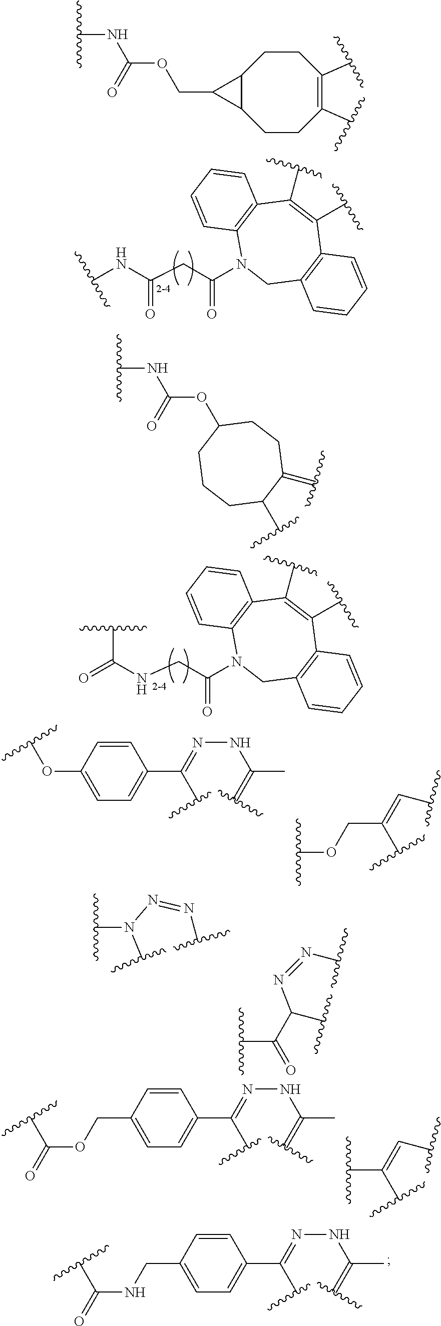
each R_{c} is chosen from —H, —CH₂—SO₃H or —SO₃H:
q is an integer ranging from 1 to 24;
each r is an integer ranging from 1 to 8;
each v is an integer ranging from 1 to 6
and the conjugate according to claim 1, of formula (B) or (C): 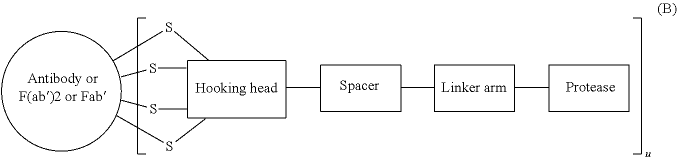
wherein u=1, 2 or 3; 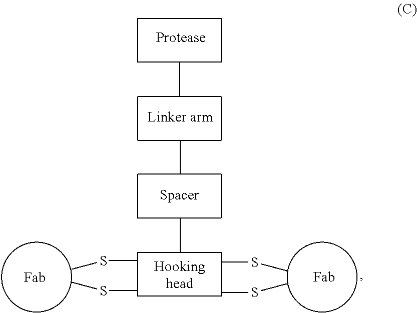
and wherein the hooking head is a compound of formula (II): 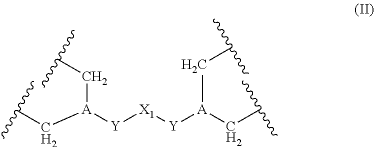
wherein:
each A is the residue of a phenyl or pyridyl;
each Y is a direct bond, —CH₂—, —O—, —S—, —CO—, —NH— or —C(═NRI)—;
X₁ is chosen from: 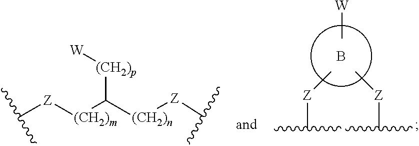 [symbol]
is a (C₃-C₆)cycloalkyl, a (C₆-C₁₀)aryl or a saturated, unsaturated or partially unsaturated heterocycle, having 5 to 15 members and comprising 1 to 4 heteroatoms chosen from nitrogen, oxygen and sulfur;
m, n and p are each, independently of one another, an integer ranging from 0 to 8;
each q is an integer ranging from 1 to 24;
each r is an integer ranging from 1 to 8;
each s is an integer ranging from 0 to 6;
each u is an integer ranging from 1 to 6;
with the exception of the following compounds:
2,6-bis[2,6-bis(bromomethyl)phenyl]benzoic acid, and
1,3-bis[[3,5-bis(bromomethyl)phenoxylmethyl]-5-prop-2-ynoxy-benzene;
each Z is a direct bond, —CH₂—, —O—, —S—, —CO—, —NH— or —C(═NR₁)—;
W is —ORₐ, —COR₂, —CONR₃R₄ or —NR₃COR₄;
Rₐ is —(CH₂CH₂O)_{q}—(CH₂)ᵣR₅-, —(CHR_{c})ᵣ-R₅-, —COR_{b}, —(CHR_{c})ᵣ—NHCO—(CH₂CH₂O)—(CH₂)ᵣ- R₅-, —(CHR_{c})ᵣ—CONH—(CH₂CH₂O)_{q}—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R_{b} is —(CH₂CH₂O)_{q}—(CH₂)ᵣR₅-, —O(CH₂CH₂O)—(CH₂)ᵣR₅-, —(CHR_{c})ᵣR₅-, —O(CHR_{c})ᵣR₅-, —(CHR_{c})ᵣ—NHCO—(CH₂CH₂O)_{q}—(CH₂)ᵣ-R₅-, —(CHR_{c})ᵣ—CONH—(CH₂CH₂O)_{q}—(CH₂)ᵣR₅-, —(CH₂CH₂O) (CH₂)ᵣ—NHCO—(CHR_{c})ᵣR₅- or —(CH₂CH₂O)—(CH₂)ᵣ—CONH—(CHR_{c})ᵣR₅-;
R₁ is —H, —OH or —(C₁-C₆)alkyl;
R₂ is —(CH₂CH₂O)_{q}—(CH₂)ᵣR₅-, —(CHR_{c})ᵣR₅-, —O(CH₂CH₂O)_{q}—(CH₂)ᵣR₅-, —O(CHR_{c})ᵣ-R₅-, —O(CHR_{c})ᵣ—NHCO—(CH₂CH₂O)_{q}—(CH₂)ᵣ-R₅-, —O(CHR_{c})ᵣ—CONH—(CH₂CH₂O)_{q}—(CH₂)ᵣ-R₅-, —O(CH₂CH₂O)—(CH₂)ᵣ—NHCO—(CHR_{c})ᵣ-R₅— or —O(CH₂CH₂O)—(CH₂)ᵣ—CONH— (CHR_{c})ᵣR₅-;
R₃ is —H, —(C₁-C₆)alkyl or —(CH₂)ᵥ—SO₃H:
R₄ is —(CH₂CH₂O)_{q}—(CH₂)ᵣR₅-, —(CHR_{c})ᵣ-R₅-, —(CHR_{c})ᵣ—NHCO—(CH₂CH₂O) (CH₂)ᵣ-R₅-, —(CHR_{c})ᵣ—CONH—(CH₂CH₂O)—(CH₂)ᵣ-R₅-, —(CH₂CH₂O)—(CH₂)ᵣ—NHCO—(CHR_{c})ᵣ-R₅— or —(CH₂CH₂O)—(CH₂)ᵣ—CONH—(CHR_{c})ᵣ-R₅-;
R₅ is chosen from: 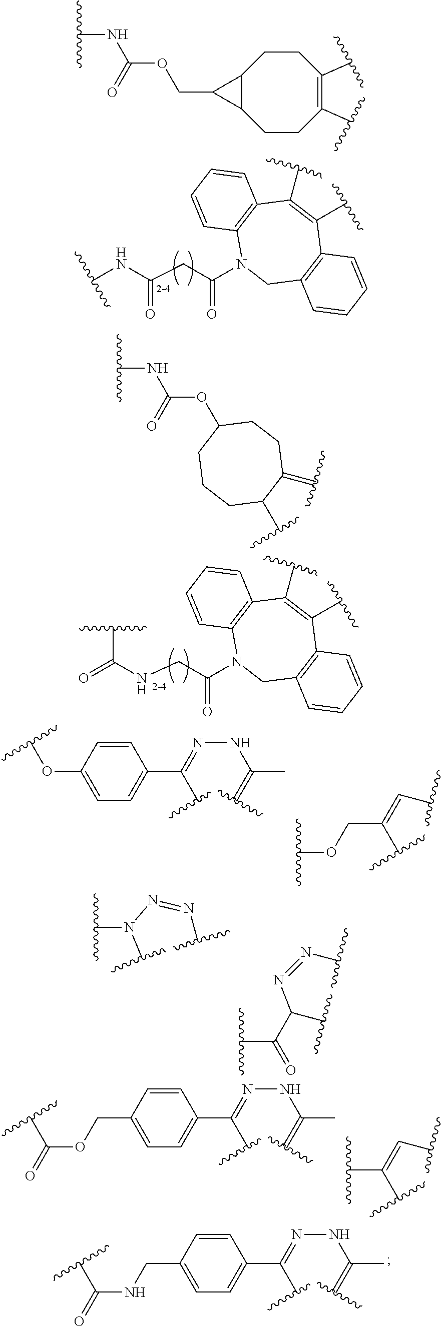
each R_{c} is independently —H, —CH₂—SO₃H or —SO₃H:
m and n are each, independently of one another, an integer ranging from 0 to 3;
p is equal to 0, 1 or 2;
each q is, independently of one another, an integer ranging from 1 to 24;
each r is, independently of one another, an integer ranging from 1 to 8;
v is an integer ranging from 1 to 6,
wherein the portion constituted by the spacer and the linker arm is represented by one of the formulas (III) or (IV):

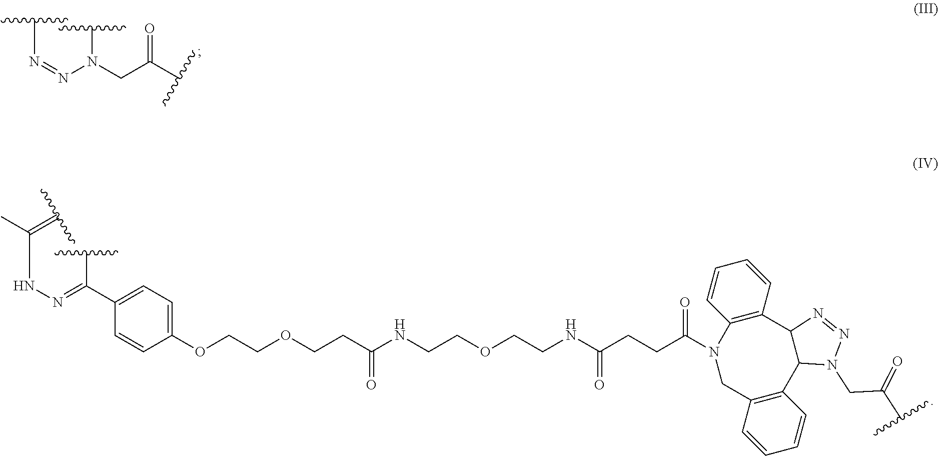 [SMILES: */N=N\N(*)CC(*)=O ; *C(=O)CN1N=NC2c3ccccc3N(C(=O)CCC(=O)NCCOCCNC(=O)CCOCCOc3ccc(/C(*)=N/NC(=*)C)cc3)Cc3ccccc3C21]

21. The conjugate according to claim 1, wherein the antibody fragment is chosen from Fab, Fab′ and F(ab′)₂.

22. The conjugate according to claim 1, wherein the protease is specific to the hinge region of IgG1, IgG2, IgG3 or IgG4.

23. The conjugate according to claim 1, wherein the protease is chosen from IdeS, IdeZ, IdeE, IgdE, SeMac.

24. The conjugate according to claim 1, of formula (V) or (VI):

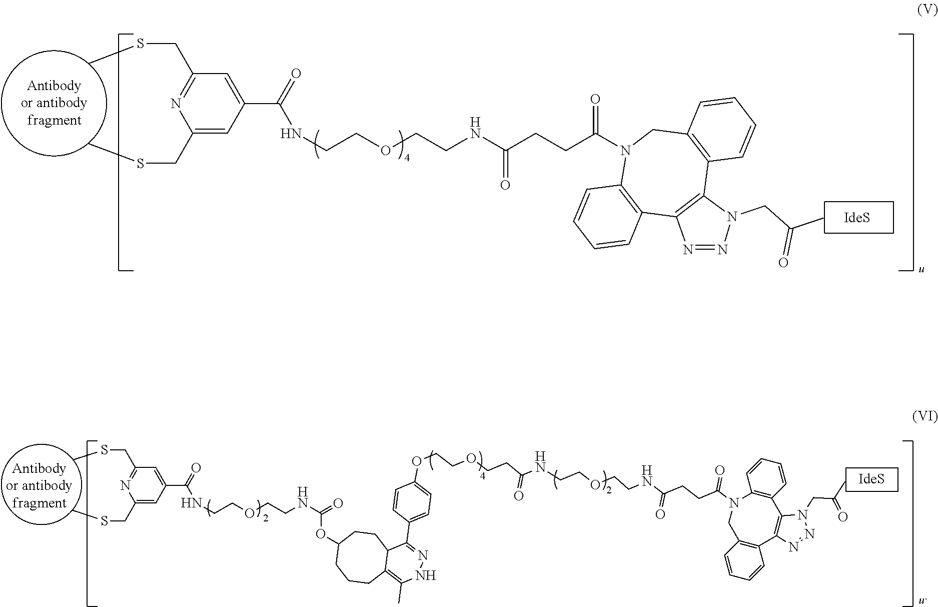

25. The conjugate according to claim 1, of formula (V′) or (VI′):

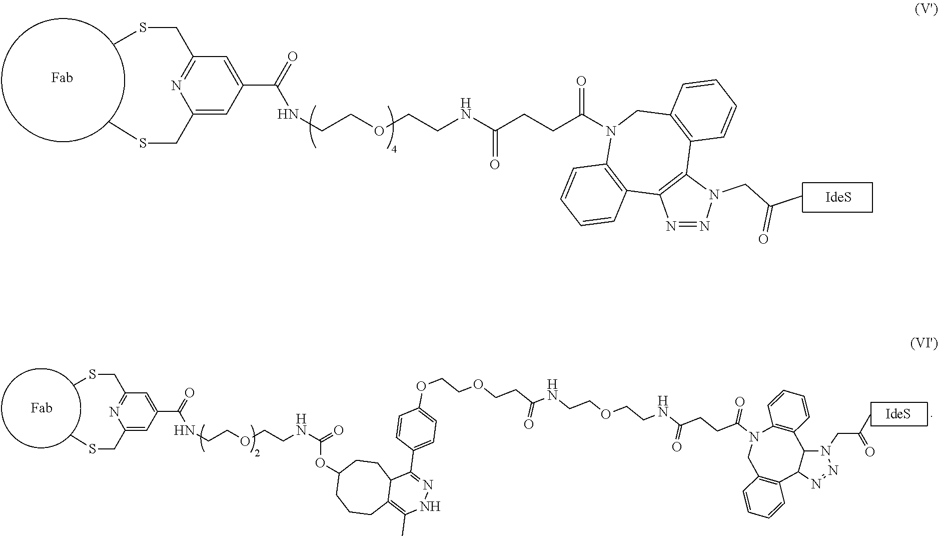

26. A composition comprising one or more conjugates according to claim 1.

27.-31. (canceled)

32. A method of treatment of a subject having a disease induced by autoantibodies comprising administering to said subject a conjugate comprising:
a) an antibody or an antibody fragment, and
b) a specific protease for the immunoglobulin G (IgG) hinge region.

33. A method of treatment of a subject having an autoimmune disease induced by Immunoglobulins G comprising administering to said subject a conjugate comprising:
a) an antibody or an antibody fragment, and
b) a specific protease for the immunoglobulin G (IgG) hinge region.

34. The method of treatment according to claim 33, wherein the autoimmune disease induced by Immunoglobulins G is chosen from immune thrombocytopenic purpura, antiphospholipid syndrome, autoimmune haemolytic anaemia, Wegener's disease, drug-induced thrombocytopenias.

35. A method of treatment of a subject having thrombotic thrombocytopenia induced by a respiratory virus comprising administering to said subject a conjugate comprising:
a) an antibody or an antibody fragment, and
b) a specific protease for the immunoglobulin G (IgG) hinge region, for use in the treatment of a thrombotic thrombocytopenia induced by a respiratory virus.

36. A method of treatment of a subject having vaccine-induced thrombotic thrombocytopenia (VITT) comprising administering to said subject a conjugate comprising:
a) an antibody or an antibody fragment, and
b) a specific protease for the immunoglobulin G (IgG) hinge region, for use in the treatment of a vaccine-induced thrombotic thrombocytopenia (VITT).


SEQUENCE LISTING (1 sequence)

SEQ ID NO: 1 - protein, 9 aa, synthetic construct
GSSHHHHHH